(19) United States
(12) Patent Application Publication
(10) Pub. No.: US 2025/0228905 A1
(43) Pub. Date: Jul. 17, 2025

(54) MICROBIAL COMMUNITIES THAT INHIBIT CLOSTRIDIOIDES DIFFICILE AND METHODS OF USING SAME

(71) Applicant: Wisconsin Alumni Research Foundation, Madison, WI (US)

(72) Inventors: Ophelia Venturelli, Chapel Hill, NC (US); Jordy Sulaiman, Madison, WI (US)

(73) Assignee: Wisconsin Alumni Research Foundation, Madison, WI (US)

(21) Appl. No.: 19/025,081

(22) Filed: Jan. 16, 2025

Related U.S. Application Data
(60) Provisional application No. 63/621,370, filed on Jan. 16, 2024.

(51) Int. Cl. A61K 35/742; A61K 9/00; A61K 45/06; A61P 31/04; C12N 1/20; C12R 1/145

(52) U.S. Cl. CPC A61K 35/742; A61K 9/0053; A61K 45/06; A61P 31/04; C12N 1/20; C12R 2001/145

(57) ABSTRACT

Provided herein are microbial communities that inhibit Clostridioides difficile and methods of using the microbial communities to inhibit C. difficile.

### CROSS-REFERENCE TO RELATED APPLICATIONS

Priority is hereby claimed to U.S. Provisional Application 63/621,370, filed Jan. 16, 2024, which is incorporated herein by reference in its entirety.

### STATEMENT REGARDING FEDERALLY SPONSORED RESEARCH

This invention was made with government support under AI159980 awarded by the National Institutes of Health. The government has certain rights to the invention.

### FIELD OF THE INVENTION

The invention is directed to microbial communities that inhibit Clostridioides difficile and methods of using the microbial communities to inhibit C. difficile.

### BACKGROUND

Clostridioides difficile is an opportunistic pathogen that causes diarrhea and colitis (inflammation of the colon) or C. difficile infection (CDI). Most cases of CDI occur during or shortly after taking antibiotics. Together, antibiotics and C. difficile substantially disrupt the normal flora (microbiome) of the gut, which normally provides colonization resistance. As a result, there is a high recurrence rate of CDI in antibiotic-treated patients, thereby limiting the options for follow-on treatment.

Options for treating CDI include monoclonal antibodies that target a specific toxin produced by C. difficile. While such treatments can be effective, they are not without their limitations, so researchers continue to pursue alternatives.

One promising alternative is fecal microbiota transplantation (FMT), whereby gut microbiota from healthy donors is transplanted into the gastrointestinal tract of a patient. This approach has the effect of reestablishing the microbiome of the patient. However, variations in microbiome compositions across FMT donors leads to uncertainty in efficacy and safety (e.g., adverse health effects including death and transfer of antibiotic resistant bacteria).

Strategies for treating CDI that avoid the aforementioned problems are needed.

### SUMMARY OF THE INVENTION

One aspect of the invention is directed to composition comprising a bacterial community.

In some embodiments, the bacterial community comprises one or more purified bacterial strains of any one or more of Bacteroides thetaiotaomicron, Bacteroides uniformis, Phocaeicola vulgatus, Clostridium hiranonis, Clostridium scindens, Collinsella aerofaciens, and Desulfovibrio piger.

In some embodiments, the bacterial community comprises a purified bacterial strain of Clostridium hiranonis.

In some embodiments, the bacterial community comprises a purified bacterial strain of each of: Clostridium hiranonis; and any one or more of Bacteroides thetaiotaomicron, Bacteroides uniformis, Phocaeicola vulgatus, Clostridium scindens, Collinsella aerofaciens, and Desulfovibrio piger.

In some embodiments, the bacterial community comprises a purified bacterial strain of each of: Clostridium hiranonis; and any one or more or two or more of Phocaeicola vulgatus, Clostridium scindens, Collinsella aerofaciens, and Desulfovibrio piger.

In some embodiments, the bacterial community comprises a purified bacterial strain of each of Clostridium hiranonis, Clostridium scindens, and Desulfovibrio piger.

In some embodiments, the composition is devoid of any bacterial strains of B. hydrogenotrophica, E. lenta, or B. hydrogenotrophica and E. lenta.

In some embodiments, the composition is devoid of any bacterial strains of Clostridium hathewayi, Blautia hansenii, Blautia producta, Blautia producta ATCC 27340, Clostridium bacterium UC5.1-1D4, Blautia coccoides, Eubacterium contortum, Eubacterium fissicatena, Sellimonas intestinalis, Dracourtella massiliensis, Dracourtella massiliensis GDI, Ruminococcus torques, Anaerostipes caccae, Clostridium scindens, Marvinbryantaformexigens, Eisenbergiella tayi, Flavinofractor plautii, Clostridium orbiscindens 1_3_50AFAA, Lachnospiraceae bacterium 7-_I_58FAA, Subdoligranulum, Anaerotruncus colihominis, Anaerotruncus colihominis DSM 17241, Clostridium symbiosum, Clostridium symbiosum WAL-14163, Clostridium bolteae, Clostridium bolteae 90A9, Dorea longicatena, Dorea longicatena CAG: 42, Clostridium innocuum, Erysipelotrichaceae_bacterium_21_3, Blautia wexlerae, Clostridium disporicum, Erysipelatoclostridium ramosum, Pseudoflavinofractor capillosus, Turicibacter sanguinis, Lactobacillus mucosae, Ruminococcus obeum, Megasphaera elsdenii, Acidaminococcus fermentans, Acidaminococcus intestine, Ruminococcus faecis, Bacteroides cellulosilyticus, Anaerostipes hadrus, Eubacterium rectale, Ruminococcus champanellensis, Ruminococcus albus, Bifidobacterium bifidum, Blautia luti, Roseburia faecis, Fusicatenibacter saccharivorans, Roseburia faecis, Blautia faecis, Dorea formicigenerans, Bacteroides ovatus, or any combination of at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, or each of the foregoing.

In some embodiments, one or more of the one or more purified bacterial strains are in spore form. In some embodiments, each of the one or more purified bacterial strains is in spore form.

In some embodiments, one or more of the one or more purified bacterial strains is in vegetative form. In some embodiments, each of the one or more purified bacterial strains is in vegetative form.

In some embodiments, each of the one or more purified bacterial strains is lyophilized.

In some embodiments, the bacterial community comprises bacterial strains purified from different sources.

In some embodiments, the bacterial community inhibits C. difficile toxin production. In some embodiments, the bacterial community inhibits C. difficile toxin production in vitro. In some embodiments, the bacterial community inhibits C. difficile toxin production in vivo. In some embodiments, the bacterial community inhibits C. difficile toxin production in vitro and in vivo.

In some embodiments, the bacterial community inhibits C. difficile replication and/or survival. In some embodiments, the bacterial community inhibits C. difficile replication and/or survival in vitro. In some embodiments, the bacterial community inhibits C. difficile replication and/or survival in vivo. In some embodiments, the bacterial community inhibits C. difficile replication and/or survival in vitro and in vivo.

In some embodiments, the composition is in the form of a pharmaceutical composition comprising the bacterial community in combination with a pharmaceutically acceptable excipient.

In some embodiments, the composition is formulated for oral delivery.

In some embodiments, the composition is formulated for rectal delivery.

In some embodiments, the composition is formulated for delivery to the intestine.

In some embodiments, the composition is formulated for delivery to the colon.

In some embodiments, the composition is in the form of a food product comprising the bacterial community and a nutrient.

Another aspect of the invention is directed to methods of treating a pathologic infection, such as a C. difficile infection, in a subject.

In some embodiments, the methods comprise administering to the subject a therapeutically effective amount of the composition of any prior claim to treat the pathogenic infection.

In some embodiments, the subject is human.

In some embodiments, the subject is an asymptomatic C. difficile carrier.

In some embodiments, the subject is administered a dose of an antibiotic prior to administration of the composition. In some embodiments, the subject is administered more than one dose of the antibiotic prior to administration of the composition. In some embodiments, the subject has not been administered an antibiotic prior to administration of the composition.

In some embodiments, the composition is administered to the subject by oral administration. In some embodiments, the composition is administered to the subject by rectal administration.

The objects and advantages of the invention will appear more fully from the following detailed description of the preferred embodiment of the invention made in conjunction with the accompanying drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1E. C. difficile isolates from diseased and healthy individuals exhibit substantial inter-strain genotypic and phenotypic variability. FIG. 1A. Schematic of approach to fit a logistic growth model to monoculture growth data of 19 C. difficile strains (including 18 C. difficile isolates from diseased and healthy individuals, Sulaiman et al. 2024 (Sulaiman J E, Thompson J, Qian Y, Vivas E I, Diener C, Gibbons S M, Safdar N, Venturelli O S. Elucidating human gut microbiota interactions that robustly inhibit diverse Clostridioides difficile strains across different nutrient landscapes. Nat Commun. 2024 Aug. 28; 15(1):7416) at Table S1) in 12 media containing different carbohydrates (Sulaiman et al. 2024 at FIG. S1a-b). In the model, xᵢ is the absolute abundance of species i, parameter rᵢ represents the maximum growth rate, and Kᵢ denotes the carrying capacity. FIG. 1B. Biclustering heatmap of the carrying capacity (Kᵢ) of C. difficile isolates. Different symbols indicate isolates from diseased patients (triangle) or healthy individuals (circle). FIG. 1C. Heatmap of presence/absence of all genes identified across the 19 C. difficile strains (pangenome). The columns indicate genes and the rows indicate C. difficile strains. Blue indicates gene presence and white indicates gene absence. FIG. 1D. Biclustering heatmap of the Average Nucleotide Identity (ANI) of C. difficile isolate pairs based on their whole-genome sequence. FIG. 1E. Scatter plot of the total growth difference (TGD) between isolate pairs and the number of orthologous genes between isolate pairs. TGD is the sum of absolute value of the difference in the AUC of pairs of strains across 12 media. Two-sided Spearman's rho and p-value are shown, which were computed using the spearman from the scipy package in Python. Source data are provided as a Source Data file. Parts of the figure are generated using Biorender.

FIGS. 2A-2G. C. difficile-preferred carbohydrates reduce the frequency of negative inter-species interactions across multiple C. difficile strains. FIG. 2A. Phylogenetic tree of a 7-member human gut community and C. difficile, generated using the 16S rRNA sequence of each species. FIG. 2B. Bipartite network of carbohydrate utilization based on monoculture growth profiles in Sulaiman et al. 2024 at FIG. S1a-b. The edge thickness indicates the difference between AUC₂₄ₕ in the presence of specific carbohydrates with the absence of carbohydrates. Only edges with a magnitude >2 are shown. For C. difficile, the average AUC values of all strains were used for calculation. FIG. 2C. Schematic of workflow to decipher interactions between different C. difficile strains and human gut bacteria (See Methods). The gLV model was fit to species absolute abundance and the inferred gLV parameters revealed inter-species interactions. FIGS. 2D-2E. Inferred inter-species interaction networks between the 7 gut species and each of the representative C. difficile strains in the presence of glucose (FIG. 2D) or the mixed carbohydrates (FIG. 2E). Node size represents species carrying capacity in monoculture and edge width represents the magnitude of the inter-species interaction coefficient (aj). Only parameters whose absolute values were significantly constrained to be non-zero based on the Wald test are shown (Sulaiman et al. 2024 at FIG. S8 and FIG. S10). Percentage of positive (blue) and negative (red) interactions for each community are shown. The heatmap shows inter-species interaction coefficients between the different C. difficile strains and the 7 gut species. FIGS. 2F-2G. Scatter plots of C. difficile absolute abundance at 24 h as a function of initial species richness in all possible 2-8 member communities simulated with the gLV model (gray) and experimental measurements (mean of biological replicates, black data points) in the presence of glucose (FIG. 2F) or mixed carbohydrates (FIG. 2G). Red dashed line indicates linear regression between the initial species richness and C. difficile absolute abundance at 24 h, with 95% confidence interval (c.i.) shown as shading. Two-sided Pearson's correlation coefficient (r) and p-values are shown, which were computed using the pearsonr from the scipy package in Python. Source data are provided as a Source Data file. Parts of the figure are generated using Biorender.

FIGS. 3A-3H. Genome-wide transcriptional profiling of C. difficile DSM27147 and C. difficile MS001 in the presence of C. scindens reveals strain-level differences in proline utilization. FIG. 3A. Model prediction and experimental validation of the relative abundance of CD-CS co-cultures, shown as mean±s.d. (n=3). p-values from unpaired t-test (two-sided) of species relative abundance are shown. FIG. 3B. Schematic of genome-wide transcriptional profiling of C. difficile DSM or MS001 in the presence of CS. Stacked bar plot (right) shows the relative abundance determined with 16S rRNA gene sequencing (mean±s.d., n=2). FIGS. 3C-3D. Volcano plots of log-transformed transcriptional fold changes for C. difficile DSM (FIG. 3C) or MS001 strain (FIG. 3D) in the presence of CS compared to their respective monoculture. Vertical dashed lines indicate 2-fold change, and the horizontal dashed line indicates the statistical significance threshold as calculated by DESeq2's Wald test with Benjamini-Hochberg multiple testing correction (BH-adjusted p value=0.05). FIG. 3E. Bar plot of the log-transformed fold changes of the proline reductase (prd) genes in C. difficile in the presence of CS compared to their respective monoculture. Adjusted p-values from DESeq2's Wald test are shown. FIG. 3F. Dose response of the growth of C. difficile DSM, MS001, or CS monoculture as a function of the initial proline concentration in the mixed carbohydrates media. AUC₄₈ₕ was calculated from the growth curves in Sulaiman et al. 2024 at FIG. S17a. Data were shown as mean and 95% c.i. (shading), n=3. Statistically significant p-values from unpaired t-test (two-sided) of the AUC₄₈ₕ between DSM and MS001 strain at specific proline concentrations are shown. FIG. 3G. Stacked bar plot of the relative abundance of C. difficile DSM or MS001 in co-culture with CS in media supplemented with different proline concentrations. Each bar represents the average relative abundance of each species, and the error bars represent 1 s.d. (n=3). p-values from unpaired t-test (two-sided) of the relative abundance at specific proline concentrations are shown. FIG. 3H. Percentage reduction of C. difficile absolute abundance in media supplemented with different proline concentrations compared to media without proline based on data from Sulaiman et al. 2024 at FIG. S17d (mean±s.d., n=3). Source data are provided as a Source Data file.

FIGS. 4A-4D. C. hiranonis inhibits the growth and toxin production of diverse C. difficile strains. FIG. 4A. Phylogenetic tree of 25-member human gut community and C. difficile. FIG. 4B. Schematic of the workflow to measure species absolute abundance to inform the gLV model and toxin concentration via ELISA. FIG. 4C. Heatmap of toxin yield (toxin production per C. difficile abundance at 24 h) of different C. difficile strains when grown in pairwise and 26-member communities with human gut bacteria in the mixed carbohydrates media. Toxin concentrations were measured in monocultures or communities after 24 h of growth using ELISA (n=3) (Sulaiman et al. 2024 at FIG. S19a). Asterisks on the heatmap indicate the p-value from unpaired t-test (two-sided) of the toxin yield in co-cultures compared to C. difficile monocultures: *** indicates p<0.001, ** indicates p<0.01, * indicates p<0.05, NT indicates no toxin. The exact p-values are available in Sulaiman et al. 2024 at Table S9. FIG. 4D. Scatter plots of the inter-species interaction coefficients (aᵢⱼ, where C. difficile is the recipient) versus toxin concentration in co-cultures. Solid data points indicate the mean of the biological replicates which are represented by transparent data points connected to the mean with transparent lines. Two-sided Spearman's rho and p-value are shown, which were computed using the spearmanr from the scipy package in Python. Parts of the figure are generated using Biorender. Source data are provided as a Source Data file.

FIGS. 5A-5G. C. hiranonis alters C. difficile metabolism and other important cellular processes. FIG. 5A. Schematic of the genome-wide transcriptional profiling of C. difficile DSM 27147 strain in the presence of CH. FIG. 5B. Volcano plot of log-transformed transcriptional fold changes for C. difficile in the presence of CH compared to monoculture. Vertical dashed lines indicate a 2-fold change, and the horizontal dashed line indicates the statistical significance threshold as calculated by DESeq2's Wald test with Benjamini-Hochberg multiple testing correction (BH-adjusted p value=0.05). FIG. 5C. Scatter plot of fold changes of C. difficile DSM 27147 differentially expressed genes in the presence of CS and CH. Only genes with p-value <0.05 are shown. Blue indicates a consistent sign of fold changes whereas red indicates an opposite sign of fold changes. Grey indicates genes that are not differentially expressed in the presence of CS or CH (less than 2-fold change marked by the dashed lines). Two-sided Spearman's rho and p-value are shown, which were computed using the spearmanr from the scipy package in Python. FIG. 5D. Differentially expressed genes that are involved in C. difficile's fermentation and energy metabolism. FIGS. 5E-5F. Bar plots of the log-transformed fold changes of selected highly differentially expressed genes of C. difficile DSM in the presence of CH. Horizontal dashed lines indicate a 2-fold change. FIG. 5G. Schematic highlighting substantial transcriptional changes in C. difficile in the presence of CH compared to CS. Parts of the figure are generated using Biorender. Source data are provided as a Source Data file.

FIGS. 6A-6E. C. hiranonis robustly ameliorates the disease severity of germ-free mice harboring different C. difficile strains or subjected to nutrient perturbations. FIG. 6A. Schematic of experimental design. One day before the experiment, the mice were fed with either unmodified drinking water or drinking water supplemented with C. difficile-preferred carbohydrates. Mice were orally gavaged with CH for one week prior to the introduction of C. difficile. We used n=5 for the mice orally gavaged with C. difficile DSM and CH with unmodified drinking water, and n=4 for all other groups of mice. FIG. 6B. Percent survival after C. difficile gavage. FIG. 6C. Percent of initial weight after C. difficile gavage. Data points indicate individual mice, and the line indicates the average of all mice in the group. The horizontal dashed line indicates the weight limit of 75%. Mice that decreased in weight below the limit were sacrificed. Statistically significant p-values from unpaired t-test (two-sided) between the weight of mice orally gavaged with CH and the CD-only group are shown. FIG. 6D. C. difficile abundance in the fecal (survived mice) and cecal content (dead mice) over time as determined by colony forming unit (CFU) counting on C. difficile selective plates. The horizontal dashed line indicates the limit of detection. Statistically significant p-values from unpaired t-test (two-sided) between the C. difficile CFU of mice orally gavaged with CH and the CD-only group are shown. FIG. 6E. Total C. difficile toxin concentration per mg of cecal content. Data were shown as mean±1 sd. Statistically significant p-values from unpaired t-test (two-sided) between the toxin concentration of mice orally gavaged with CH and the CD-only group are shown. In FIGS. 6B-6E, top figures show the mice group that was inoculated with C. difficile DSM and provided unmodified drinking water, middle figures show the mice group that was orally gavaged with C. difficile DSM and provided with drinking water supplemented with C. difficile-preferred carbohydrates, and bottom show the mice orally gavaged with C. difficile MS008 and provided with unmodified drinking water. Parts of the figure are generated using Biorender. Source data are provided as a Source Data file.

FIG. 7. Middle-richness communities containing C. hiranonis with similar or better inhibitory activity against C. difficile compared to C. hiranonis alone. A-B. C. difficile absolute abundance at 24 h (A) and toxin fold change compared to monoculture as quantified by ELISA (B) in different community combinations when grown in the mixed carbohydrates media. Different colors indicate different communities. Horizontal dashed lines indicate C. difficile absolute abundance in the CD-CH pair (A) or toxin fold change in the CD-CH pair compared to monoculture (B). Statistically significant p-values from unpaired t-test (two-sided) between C. difficile absolute abundance or toxin fold change in specific middle-richness community and C. difficile absolute abundance or toxin fold change in CD-CH pair are shown.

FIGS. 8A-8C. Model could distinguish strong inhibitory community (SIC) and non-inhibitory community (NIC) against C. difficile. FIG. 8A. Extent of C. difficile inhibition by all possible 2-8-member community combinations as predicted by the gLV model trained on the mixed carbohydrates media. Two communities were selected for follow-up analyses: Non-Inhibitory Community (NIC) which consists of BU, CA, and DP, and Strong Inhibitory Community (SIC) which consists of CH, CS, and DP. FIG. 8B. Inferred interspecies interaction networks between the gut species in the NIC (left) or SIC (right) and each of the representative C. difficile strains in the mixed carbohydrates media. Node size represents species carrying capacity in monoculture (mean of all biological replicates) and edge width represents the magnitude of the interspecies interaction coefficient (aᵢⱼ). Edges shows parameters whose absolute values were significantly constrained to be non-zero based on the Wald test. FIG. 8C. Species absolute abundance experimentally measured in the absence and presence of different C. difficile strains in the mixed carbohydrates media after 24 h of growth. C. difficile monoculture growth after 24 h is shown for comparison. Each bar represents the average absolute abundance of each species (n=3). Error bars represent the standard deviation of 3 biological replicates. p-values from unpaired t-test (two-sided) of the C. difficile absolute abundance in the SIC versus NIC are shown.

FIG. 9. Composition of bacterial species in mice over time. A-C. Relative abundance of C. difficile and CH in the mice gavaged with C. difficile DSM fed with normal drinking water (A), mice gavaged with C. difficile DSM fed with C. difficile-preferred carbohydrates supplemented drinking water (B), and mice gavaged with C. difficile MS008 fed with normal drinking water (C) as determined by 16S sequencing. D. Relative abundance of C. difficile, BU, CA, and DP in the mice gavaged with C. difficile DSM fed with normal drinking water as determined by 16S sequencing. Datapoints represent individual mice, and the line represents the average of all mice in the group. Parts of the figure are generated using Biorender.

### DETAILED DESCRIPTION OF THE INVENTION

Disclosed herein are compositions comprising bacterial communities. The bacterial communities can comprise purified bacterial strains. In some embodiments, the compositions are in the form of pharmaceutical compositions comprising the bacterial communities. In some embodiments, the compositions are in the form of food products comprising the bacterial communities. Also disclosed are methods of treating a pathogenic infection, such as Clostridium difficile (C. difficile) infection, in a subject by administering the compositions to the subject.

Various factors including antibiotic usage can induce dysbiosis of the gastrointestinal tract, which may allow for colonization by pathogenic microorganisms, such as C. difficile. Such colonization or pathogenic infection can lead to a variety of adverse effects in the subject including diarrhea, which is one of the primary symptoms characteristic of C. difficile infection (CDI). In the case of CDI, diarrhea is thought to be a result of C. difficile production of Toxin B (also referred to as cytotoxin TcdB), which results in opening of the tight junctions between intestinal epithelial cells, increasing vascular permeability, hemorrhage, and inflammation.

The compositions described herein are effective in the treatment of C. difficile infection. As shown herein, the disclosed compositions are effective in suppressing the pathogenic effects of C. difficile infection. The compositions provided herein can reduce the amount of C. difficile after infection and thereby provide an effective method for eliminating C. difficile from the body (e.g., the gut). The compositions disclosed herein have been found to reduce or inhibit production or activity of C. difficile Toxin B and thereby represent effective compositions for the treatment or prevention of CDI. The compositions disclosed herein have also been found to inhibit the growth and/or survival of C. difficile.

The present disclosure provides compositions comprising bacterial communities of purified bacterial strains that can be administered to subjects experiencing or having experienced a pathogenic infection to treat the infection. In some embodiments, the compositions may be administered to subjects who may be at risk for a pathogenic infection. Such subjects include subjects who previously had pathogenic infections, subjects who have been treated with antibiotics, and subjects who will undergo a procedure that will put them at an increased risk for a pathogenic infection (e.g., surgery and/or hospitalization). In some embodiments, the pathogenic infection, is infection by a pathogen that is present predominantly in the gut or the intestine. In some embodiments, the pathogen that is present predominantly in the gut or the intestine is Clostridium difficile. In some embodiments, the one or more of the bacterial strains of the compositions provided herein colonize or recolonize the intestinal tract or parts of the intestinal tract (e.g., the colon or the cecum) of the subject. Such colonization or recolonization may also be referred to as grafting. In some embodiments, the one or more of the bacterial strains of the compositions recolonize the intestinal tract (e.g., the colon or the cecum) of the subject after the naturally present microbiome has been partially or completely removed, e.g., because of administration of antibiotics. In some embodiments, the one or more of the bacterial strains of the compositions colonize a dysbiotic gastrointestinal tract.

In some embodiments, the one or more of the bacterial strains of the compositions can “outgrow” a pathogen, such as C. difficile. Thus, in some embodiments, if a pathogen (e.g., C. difficile) and one or more bacteria of compositions provided herein are both present in the intestinal tract (e.g., the colon or the cecum), the one or more bacteria of compositions provided herein grow faster (e.g., have a shorter doubling time) than the pathogen, thereby preventing the pathogen from accumulating in the intestinal tract (e.g., the colon or the cecum). In some embodiments, the faster growth results because the one or more bacteria of the compositions provided herein are better at grafting in the intestinal tract (e.g., the colon or the cecum). In some embodiments, the faster growth results because the one or more bacteria of the compositions provided herein are better at metabolizing nutrients present in the intestinal tract (e.g., the colon or the cecum). In some embodiments, the compositions of bacterial strains provided herein prevent or inhibit production of bacterial toxins by the pathogenic infection, or prevent or inhibit the cytopathic or cytotoxic effects of such bacterial toxins.

In some embodiments, the bacterial strains of the compositions provided herein can treat pathogenic infections because of the synergy between the bacterial strains. Thus, without being limiting, in some embodiments, the combination of the bacterial strains of the compositions provided herein act synergistically because the combination of the strains is particularly well-suited to use nutrients in the intestinal tract (e.g., the colon or the cecum), for instance through metabolic interactions, and/or because the combination is superior in grafting (e.g., by providing a favorable microenvironment).

In some embodiments, a pathogenic infection such as C. difficile is treated because the combination of bacterial strains of the compositions provided herein is superior in the use of nutrients when compared to the pathogen such as C. difficile, thereby suppressing the growth of the pathogen such as C. difficile. In some embodiments, a pathogenic infection such as C. difficile is treated because the combination of bacterial strains of the compositions provided herein is superior in grafting when compared to the pathogen such as C. difficile, thereby suppressing the growth of the pathogen such as C. difficile. In some embodiments, a pathogenic infection such as C. difficile is treated because the combination of bacterial strains of the compositions provided herein is superior in the use of nutrients and in grafting when compared to the pathogen such as C. difficile, thereby suppressing the growth of the pathogen such as C. difficile. In some embodiments, a pathogenic infection such as C. difficile is treated because the combination of bacterial strains of the compositions provided herein inhibits the growth and/or survival of the pathogen such as C. difficile. In some embodiments, a pathogenic infection such as C. difficile is treated because the combination of bacterial strains of the compositions provided herein inhibits the growth and/or survival of the pathogen and induces regulatory T cells (Tregs) in the subject that results in reduction or elimination of the pathogen such as C. difficile.

In some embodiments, the synergistic effect is provided by the capacity of the combination to colonize specific niches in the intestinal tract (e.g., the colon or the cecum). In some embodiments, the synergistic effect is provided by the capacity of the combination to metabolize specific nutrients. In some embodiments, the synergistic effect is provided by the capacity of the combination to provide specific metabolites to the environment. Such specific metabolites may suppress growth of the pathogen and/or stimulate growth of non-pathogens. In some embodiments, the synergistic effect is provided by the capacity of the combination to provide short-chain fatty acids to the environment. In some embodiments, the synergistic effect is provided by the capacity of the combination to provide specific short-chain fatty acids to the environment. In some embodiments, the synergistic effect is provided by the capacity of the combination to produce butyrate. In some embodiments, the synergistic effect is provided by the capacity of the combination to produce acetate. In some embodiments, the synergistic effect is provided by the capacity of the combination to produce lactate. In some embodiments, the synergistic effect is provided by the capacity of the combination to produce propionate. In some embodiments, the synergistic effect is provided by the capacity of the combination to produce succinate. In some embodiments, the synergistic effect is provided by the capacity of the combination to produce multiple metabolites. In some embodiments, the synergistic effect is provided by the capacity of the combination to produce multiple short-chain fatty acids. In some embodiments, the synergistic effect is provided by the capacity of the combination to produce both butyrate and acetate. In some embodiments, the synergistic effect is provided by the capacity of the combination to produce both butyrate and lactate. In some embodiments, the synergistic effect is provided by the capacity of the combination to produce both butyrate and propionate. In some embodiments, the synergistic effect is provided by the capacity of the combination to produce both butyrate and succinate. In some embodiments, the synergistic effect is provided by the capacity of the combination to produce butyrate, acetate and additional short-chain fatty acids.

In some embodiments, the bacterial strains used in the compositions provided herein are isolated from the microbiome of healthy individuals. In some embodiments, the compositions include strains originating from a single individual. In some embodiments, the compositions include strains originating from multiple individuals. In some embodiments, the bacterial strains are obtained from multiple individuals, isolated, and grown up individually. The bacterial compositions that are grown up individually may subsequently be combined to provide the compositions of the disclosure. It should be appreciated that the origin of the bacterial strains of the compositions provided herein is not limited to the human microbiome from a healthy individual. In some embodiments, the bacterial strains originate from a human with a microbiome in dysbiosis. In some embodiments, the bacterial strains originate from non-human animals or the environment (e.g., soil or surface water). In some embodiments, the combinations of bacterial strains provided herein originate from multiple sources (e.g., human and non-human animals). In some embodiments, the compositions comprise bacterial strains purified from different sources. The bacterial strains purified from different sources can be combined together after being purified from their respective sources. The bacterial strains purified from different sources can be grown before being combined together. The bacterial strains purified from different sources can include any two, any three, any four, any five, any six, any seven, or more of the bacterial strains described herein as being included in the compositions of the invention.

In one aspect, the disclosure provides compositions comprising one or more bacterial strains. The one or more bacterial strains can comprise a strain of any one or more of Bacteroides thetaiotaomicron, Bacteroides uniformis, Phocaeicola vulgatus, Clostridium hiranonis, Clostridium scindens, Collinsella aerofaciens, and Desulfovibrio piger. In some embodiments, the one or more bacterial strains comprises a strain of Clostridium hiranonis.

In some embodiments, the one or more bacterial strains comprise two or more bacterial strains. In some embodiments, the two or more bacterial strains comprise a strain of each of: Clostridium hiranonis; and one or more, two or more, three or more, four or more, or each of Bacteroides thetaiotaomicron, Bacteroides uniformis, Phocaeicola vulgatus, Clostridium scindens, Collinsella aerofaciens, and Desulfovibrio piger. In some embodiments, the two or more bacterial strains comprise a strain of each of: Clostridium hiranonis; and one or more, two or more, three or more, or each of Phocaeicola vulgatus, Clostridium scindens, Collinsella aerofaciens, and Desulfovibrio piger. In some embodiments, the two or more bacterial strains comprise a strain of each of Clostridium hiranonis and Bacteroides thetaiotaomicron. In some embodiments, the two or more bacterial strains comprise a strain of each of Clostridium hiranonis and Bacteroides uniformis. In some embodiments, the two or more bacterial strains comprise a strain of each of Clostridium hiranonis and Phocaeicola vulgatus. In some embodiments, the two or more bacterial strains comprise a strain of each of Clostridium hiranonis and Clostridium scindens. In some embodiments, the two or more bacterial strains comprise a strain of each of Clostridium hiranonis and Collinsella aerofaciens. In some embodiments, the two or more bacterial strains comprise a strain of each of Clostridium hiranonis and Desulfovibrio piger. In some embodiments, the two or more bacterial strains comprise a strain of each of Clostridium hiranonis, Clostridium scindens, and Desulfovibrio piger.

In some embodiments, the composition is devoid of any strains of Bacteroides thetaiotaomicron. In some embodiments, the composition is devoid of any strains of Bacteroides uniformis. In some embodiments, the composition is devoid of any strains of Phocaeicola vulgatus. In some embodiments, the composition is devoid of any strains of Clostridium scindens. In some embodiments, the composition is devoid of any strains of Collinsella aerofaciens. In some embodiments, the composition is devoid of any strains of Desulfovibrio piger.

In some embodiments, the composition is devoid of any strains of B. hydrogenotrophica, E. lenta, or B. hydrogenotrophica and E. lenta. In some embodiments, the composition is devoid of any strains of B. hydrogenotrophica. In some embodiments, the composition is devoid of any strains of E. lenta. In some embodiments, the composition is devoid of any strains of B. hydrogenotrophica and E. lenta.

In some embodiments, the composition is devoid of any strains of Clostridium hathewayi, Blautia hansenii, Blautia producta, Blautia producta ATCC 27340, Clostridium bacterium UC5.1-1D4, Blautia coccoides, Eubacterium contortum, Eubacterium fissicatena, Sellimonas intestinalis, Dracourtella massiliensis, Dracourtella massiliensis GDI, Ruminococcus torques, Anaerostipes caccae, Clostridium scindens, Marvinbryantaformexigens, Eisenbergiella tayi, Flavinofractor plautii, Clostridium orbiscindens 1_3_50AFAA, Lachnospiraceae bacterium 7-_I_58FAA, Subdoligranulum, Anaerotruncus colihominis, Anaerotruncus colihominis DSM 17241, Clostridium symbiosum, Clostridium symbiosum WAL-14163, Clostridium bolteae, Clostridium bolteae 90A9, Dorea longicatena, Dorea longicatena CAG: 42, Clostridium innocuum, Erysipelotrichaceae_bacterium_21_3, Blautia wexlerae, Clostridium disporicum, Erysipelatoclostridium ramosum, Pseudoflavinofractor capillosus, Turicibacter sanguinis, Lactobacillus mucosae, Ruminococcus obeum, Megasphaera elsdenii, Acidaminococcus fermentans, Acidaminococcus intestine, Ruminococcus faecis, Bacteroides cellulosilyticus, Anaerostipes hadrus, Eubacterium rectale, Ruminococcus champanellensis, Ruminococcus albus, Bifidobacterium bifidum, Blautia luti, Roseburia faecis, Fusicatenibacter saccharivorans, Roseburia faecis, Blautia faecis, Dorea formicigenerans, Bacteroides ovatus, or any combination of at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, or each of the foregoing.

In some embodiments, the composition is devoid of any strains of Clostridium hathewayi. In some embodiments, the composition is devoid of any strains of Blautia hansenii. In some embodiments, the composition is devoid of any strains of Blautia producta. In some embodiments, the composition is devoid of any strains of Blautia producta ATCC 27340. In some embodiments, the composition is devoid of any strains of Clostridium bacterium UC5.1-1D4. In some embodiments, the composition is devoid of any strains of Blautia coccoides. In some embodiments, the composition is devoid of any strains of Eubacterium contortum. In some embodiments, the composition is devoid of any strains of Eubacterium fissicatena. In some embodiments, the composition is devoid of any strains of Sellimonas intestinalis. In some embodiments, the composition is devoid of any strains of Dracourtella massiliensis. In some embodiments, the composition is devoid of any strains of Dracourtella massiliensis GDI. In some embodiments, the composition is devoid of any strains of Ruminococcus torques. In some embodiments, the composition is devoid of any strains of Anaerostipes caccae. In some embodiments, the composition is devoid of any strains of Clostridium scindens. In some embodiments, the composition is devoid of any strains of Marvinbryantaformexigens. In some embodiments, the composition is devoid of any strains of Eisenbergiella tayi. In some embodiments, the composition is devoid of any strains of Flavinofractor plautii. In some embodiments, the composition is devoid of any strains of Clostridium orbiscindens 1_3_50AFAA. In some embodiments, the composition is devoid of any strains of Lachnospiraceae bacterium 7-_I_58FAA. In some embodiments, the composition is devoid of any strains of Subdoligranulum. In some embodiments, the composition is devoid of any strains of Anaerotruncus colihominis. In some embodiments, the composition is devoid of any strains of Anaerotruncus colihominis DSM 17241. In some embodiments, the composition is devoid of any strains of Clostridium symbiosum. In some embodiments, the composition is devoid of any strains of Clostridium symbiosum WAL-14163. In some embodiments, the composition is devoid of any strains of Clostridium bolteae. In some embodiments, the composition is devoid of any strains of Clostridium bolteae 90A9. In some embodiments, the composition is devoid of any strains of Dorea longicatena. In some embodiments, the composition is devoid of any strains of Dorea longicatena CAG: 42. In some embodiments, the composition is devoid of any strains of Clostridium innocuum. In some embodiments, the composition is devoid of any strains of Erysipelotrichaceae_bacterium_21_3. Blautia wexlerae. In some embodiments, the composition is devoid of any strains of Clostridium disporicum. In some embodiments, the composition is devoid of any strains of Erysipelatoclostridium ramosum. In some embodiments, the composition is devoid of any strains of Pseudoflavinofractor capillosus. In some embodiments, the composition is devoid of any strains of Turicibacter sanguinis. In some embodiments, the composition is devoid of any strains of Lactobacillus mucosae. In some embodiments, the composition is devoid of any strains of Ruminococcus obeum. In some embodiments, the composition is devoid of any strains of Megasphaera elsdenii. In some embodiments, the composition is devoid of any strains of Acidaminococcus fermentans. In some embodiments, the composition is devoid of any strains of Acidaminococcus intestine. In some embodiments, the composition is devoid of any strains of Ruminococcus faecis. In some embodiments, the composition is devoid of any strains of Bacteroides cellulosilyticus. In some embodiments, the composition is devoid of any strains of Anaerostipes hadrus. In some embodiments, the composition is devoid of any strains of Eubacterium rectale. In some embodiments, the composition is devoid of any strains of Ruminococcus champanellensis. In some embodiments, the composition is devoid of any strains of Ruminococcus albus. In some embodiments, the composition is devoid of any strains of Bifidobacterium bifidum. In some embodiments, the composition is devoid of any strains of Blautia luti. In some embodiments, the composition is devoid of any strains of Roseburia faecis. In some embodiments, the composition is devoid of any strains of Fusicatenibacter saccharivorans. In some embodiments, the composition is devoid of any strains of Roseburia faecis. In some embodiments, the composition is devoid of any strains of Blautia faecis. In some embodiments, the composition is devoid of any strains of Dorea formicigenerans. In some embodiments, the composition is devoid of any strains of Bacteroides ovatus.

In some embodiments, the compositions described herein comprise spore forming and non-spore forming bacterial strains. In some embodiments, the compositions described herein comprise spore forming bacterial strains. In some embodiments, the compositions described herein comprise only spore forming bacterial strains. In some embodiments, the compositions described herein comprise only non-spore forming bacterial strains. The spore-forming bacteria can be in spore form (i.e., as spores) or in vegetative form (i.e., as vegetative cells). In spore form, bacteria are generally more resistant to environmental conditions, such as heat, acid, radiation, oxygen, chemicals, and antibiotics. In contrast, in the vegetative state or actively growing state, bacteria are more susceptible to such environmental conditions, compared to in the spore form. In general, bacterial spores are able to germinate from the spore form into a vegetative/actively growing state, under appropriate conditions. For instance, bacteria in spore form may germinate when they are introduced in the intestine.

In some embodiments, at least one (e.g., 1, 2, 3, 4, 5, or more) of the bacterial strains in the composition is a spore former. In some embodiments, at least one (e.g., 1, 2, 3, 4, 5, or more) of the bacterial strains in the composition is in spore form. In some embodiments, at least one (e.g., 1, 2, 3, 4, 5, or more) of the bacterial strains in the composition is a non-spore former. In some embodiments, at least one (e.g., 1, 2, 3, 4, 5, or more) of the bacterial strains in the composition is in vegetative form (as discussed above, spore forming bacteria can also be in vegetative form). In some embodiments, at least one (e.g., 1, 2, 3, 4, 5, or more) of the bacterial strains in the composition is in spore form and at least one (e.g., 1, 2, 3, 4, 5, or more) of the bacterial strains in the composition is in vegetative form. In some embodiments, at least one bacterial strain that is considered able to form spores (i.e., a spore-former) but is present in the composition in vegetative form. In some embodiments, at least one bacterial strain that is considered able to form spores is present in the composition both in spore form and in vegetative form.

In some embodiments, the disclosure provides compositions wherein the compositions comprise bacterial strains that are spore forming bacterial strains. In some embodiments, the disclosure provides compositions wherein the compositions comprise bacterial strains that are non-spore forming bacterial strains. In some embodiments, the disclosure provides compositions wherein the compositions comprise bacterial strains that are spore forming bacterial strains and bacterial strains that are non-spore forming bacterial strains. In some embodiments, the disclosure provides compositions, wherein the compositions comprise a mixture of bacterial strains wherein at least 10% of the bacterial strains are spore forming bacterial strains, at least 20% of the bacterial strains are spore forming bacterial strains, at least 30% of the bacterial strains are spore forming bacterial strains, at least 40% of the bacterial strains are spore forming bacterial strains, at least 50% of the bacterial strains are spore forming bacterial strains, at least 60% of the bacterial strains are spore forming bacterial strains, at least 70% of the bacterial strains are spore forming bacterial strains, at least 80% of the bacterial strains are spore forming bacterial strains, at least 90% of the bacterial strains are spore forming bacterial strains. In some embodiments, up to 100% of the bacterial strains are spore forming bacterial strains. Whether a bacterial strain is a spore forming strain can be determined for instance by evaluating the genome of the bacterial strain for the presence of sporulation genes. However, it should be appreciated that not all bacteria that are predicted to encode spore forming genes can be made to sporulate. In addition, whether a bacterial strain is a spore forming strain can be determined by exposing the bacterial strain to stress conditions, e.g., heat or exposure to chemicals (e.g., ethanol or chloroform), that are known to induce sporulation.

In some embodiments of the compositions provided herein, the spore forming bacteria are in spore form. In some embodiments of the compositions provided herein, the spore forming bacteria are in vegetative form. In some embodiments of the compositions provided herein, the spore forming bacteria are both present in spore form and in vegetative form. In some embodiments, the disclosure provides compositions, wherein the compositions comprise spore forming bacteria at least 10% of the spore forming bacteria are in spore form, at least 20% of the spore forming bacteria are in spore form, at least 30% of the spore forming bacteria are in spore form, at least 40% of the spore forming bacteria are in spore form, at least 50% of the spore forming bacteria are in spore form, at least 60% of the spore forming bacteria are in spore form, at least 70% of the spore forming bacteria are in spore form, at least 80% of the spore forming bacteria are in spore form, at least 90% of the spore forming bacteria are in spore form, up to 100% in spore form.

It is envisioned that the bacterial strains of the compositions provided herein are alive and will be alive when they reach the target area (e.g., the intestines). Bacterial spores are considered to be alive in this regard. In some embodiments, bacteria that are administered as spores may germinate in the target area (e.g., the intestines). It should further be appreciated that not all of the bacteria are alive and the compositions can include a percentage (e.g., by weight) that is not alive. In addition, in some embodiments, the compositions include bacterial strains that are not alive when administered or at the time when the composition reaches the target area (e.g., the intestines). It is envisioned that non-living bacteria may still be useful by providing some nutrients and metabolites for the other bacterial strains in the composition.

Methods of inducing sporulation of spore-forming bacterial strains are well known in the art (See e.g., Paredes-Sabja et al., Trends Microbiol. (2011) 19(2):85-94). Generally, bacterial strains that are spore-formers can be made to go into spore form by stressing the bacterial strains. Non-limiting examples of stresses that can induce sporulation are an increase in temperature, change in the nutrients available and/or exposure to chemicals (e.g., ethanol or chloroform). It should be noted that bacteria that are non-spore formers, for instance because they are missing sporulation genes, cannot be made to sporulate by stress. To prepare compositions in which all the bacterial strains are in the spore form, the composition or bacterial cultures used to prepare the composition may be subjected to treatment to kill any bacteria not in spore form (e.g., in vegetative form), for example by exposing the composition to heat and are chemically breaking down the non-spore bacteria. The bacteria in spore form can subsequently be separated from the non-spore bacteria for instance by filtration.

The amount of spores can be quantified using techniques known in the art. These techniques include phase contrast microscopy for enumerating spores using a hemocytometer. In addition, the viability of spores can be determined by plating the spores and growing the spores. For instance, spores can be plated in appropriate media and incubated in the anaerobic chamber for a period of time (e.g., 48-96 hrs.). Viability can subsequently be determined by quantifying the colony forming units which correspond to spores that germinated. For instance, spores can be plated on TCCFA plates (Taurocholate, cycloserine, cefoxintin, fructose agar plates), in which taurocholate helps the spores to germinate. In addition, spores can be quantified using the dipicolinic assay (DPA assay). DPA is an agent that allows for spore selection and is a clear indicator of endospores. When complexed with terbium, bright green luminescence is observed.

In any of the compositions provided herein, in some embodiments, the bacterial strains are purified. In any of the compositions provided herein, in some embodiments, the bacterial strains are isolated. Any of the bacterial strains described herein may be isolated and/or purified, for example, from a source such as a culture or a microbiota sample (e.g., fecal matter). The bacterial strains used in the compositions provided herein can be isolated from the microbiome of healthy individuals. However, bacterial strains can also be isolated from individuals that are considered not to be healthy. In some embodiments, the compositions include strains originating from multiple individuals or sources.

As used herein, the term “isolated” bacteria that have been separated from one or more undesired component, such as another bacterium or bacterial strain, one or more component of a growth medium, and/or one or more component of a sample, such as a fecal sample. In some embodiments, the bacteria are substantially isolated from a source such that other components of the source are not detected.

As also used herein, the term “purified” refers to a bacterial strain or composition comprising such that has been separated from one or more components, such as contaminants. In some embodiments, the bacterial strain is substantially free of contaminants. In some embodiments, one or more bacterial strains of a composition may be independently purified from one or more other bacteria produced and/or present in a culture or a sample containing the bacterial strain. In some embodiments, a bacterial strain is isolated or purified from a sample and then cultured under the appropriate conditions for bacterial replication, e.g., under anaerobic culture conditions. The bacteria that is grown under appropriate conditions for bacterial replication can subsequently be isolated/purified from the culture in which it is grown.

Aspects of the present disclosure are related to methods for treating a pathogenic infection in a subject by administering a therapeutically effective amount of any of the compositions described herein. In some embodiments, the subject is a mammalian subject, such as a human, non-human primate, rodent, rabbit, sheep, pig, dog, cat, horse, or cow. In some embodiments, the subject is a human subject. In some embodiments, the subject is a carrier of a pathogenic organism and is suffering from the effects of the infection (e.g., diarrhea caused by C. difficile toxins). In some embodiments the subject is an asymptomatic carrier of a pathogen. In some embodiments, the subject is a carrier of C. difficile. In some embodiments the subject is an asymptomatic C. difficile carrier. In some embodiments, the subject has experienced recurrent or chronic pathogenic infections. In some embodiments, the subject is suffering from a first occurrence of a particular pathogenic infection. In some embodiments, the subject has been treated with antibiotics which resulted in the recurrence of the pathogenic infection. In some embodiments, the subject has been treated with antibiotics which resulted in a first occurrence of a pathogenic infection. In some embodiments, the subject is to undergo a procedure that puts the subject at a higher risk of infection. In some embodiments, the compositions provided herein are administered to a subject to lower the risk of becoming infected by a pathogen.

In some embodiments, the compositions provided herein are administered to a subject if the subject has a dysbiosis (e.g., has as microbiome associated with a disease state). In some embodiments, treatment with the compositions provided herein results in the change in the microbiome of the subject. In some embodiments, treatment with the compositions provided herein removes the dysbiosis in the subject resulting in a healthy microbiome. In some embodiments, treatment with the compositions provided herein removes the dysbiosis in the subject resulting in microbiome refractory or less susceptible to infection by a pathogen.

As used herein, the term “pathogen” in regard to a pathogenic infection refers to a microorganism (e.g., a bacterium) that causes a disease or a disease state in a subject. In some embodiments, the disease or disease state of the subject may include symptoms such as colitis, diarrhea, watery diarrhea, abdominal cramping, fever, blood or pus in the stool, nausea, dehydration, loss of appetite, chills, weight loss, and/or kidney failure. In some embodiments, the pathogenic infection may be diagnosed, for example, by detecting a pathogen (or protein or nucleic acid associated with a pathogen) in a fecal sample collected from the subject. In some embodiments, the pathogenic infection may be diagnosed, for example, by comparing the microbiota of a fecal sample of the subject with the microbiota in a fecal sample of a healthy subject.

In some embodiments, the pathogenic infection is C. difficile. In some embodiments, the C. difficile is an antibiotic-resistant C. difficile, e.g., fluoroquinolone resistant C. difficile.

Additional non-limiting examples of pathogens responsible for pathogenic infection that can be treated according to the methods provided herein can include Clostridium perfringens, Clostridium botulinum, Clostridium tributrycum, Clostridium sporogenes, Escherichia coli, Pseudomonas aeruginosa, such as multidrug-resistant Pseudomonas aeruginosa, vancomycin-resistant Enterococci (VRE) carbapenem-resistant Enterobacteriaceae (CRE) Neisseria gonorrheae, Acinetobacter, multidrug-resistant Acinetobacter, Campylobacter, multidrug-resistant Campylobacter, Candida, Fluconazole-resistant Candida, extended spectrum beta-lactamase (ESBL) producing Enterobacteriaceae, Salmonella, Salmonella Typhimurium, drug-resistant non-typhoid Salmonella spp., drug-resistant Salmonella Typhi, drug-resistant Shigella, Staphylococcus aureus, such as methicillin-resistant S. aureus or vancomycin-resistant S. aureus, drug-resistant Streptococcus pneumoniae, drug-resistant Tuberculosis, erythromycin-resistant Group A Streptococcus, clindamycin-resistant Group B Streptococcus, vancomycin-resistant Enterococci., and any combination thereof. Additional non-limiting examples of pathogens responsible for pathogenic infection that can be treated according to the methods provided herein can include Leishmania, Staphylococcus epidermis, Staphylococcus saprophyticus, Streptococcus pyogenes, Streptococcus pneumoniae, Streptococcus agalactiae, Enterococcus faecalis, Corynebacterium diptheriae, Bacillus anthracis, Listeria monocytogenes, Clostridium perfringens, Clostridium tetanus, Clostridium botulinum, Clostridium difficile, Neisseria meningitidis, Neisseria gonorrhoeae, Escherichia coli, Salmonella typhimurium, Salmonella cholerasuis, Salmonella enterica, Salmonella enteriditis, Yersinia pestis, Yersinia pseudotuberculosis, Yersinia enterocolitica, Vibrio cholerae, Campylobacter jejuni, Campylobacter fetus, Helicobacter pylori, Pseudomonas aeruginosa, Pseudomonas mallei, Haemophilus influenzae, Bordetella pertussis, Mycoplasma pneumoniae, Ureaplasma urealyticum, Legionella pneumophila, Treponema pallidum, Leptospira interrogans, Borrelia burgdorferi, Mycobacterium tuberculosis, Mycobacterium leprae, Chlamydia psittaci, Chlamydia trachomatis, Chlamydia pneumoniae, Rickettsia ricketsii, Rickettsia akari, Rickettsia prowazekii, Brucella abortus, Brucella melitens, Brucella suis, Francisella tularensis, and any combination thereof. In general, any bacterium that is capable of inducing a disease in a subject and/or that is not present in healthy individual is considered a pathogen herein. It should be appreciated that a subject may carry multiple pathogens and/or have multiple pathogenic infections.

Any of the compositions described herein may be administered to a subject in a therapeutically effective amount or a dose of a therapeutically effective amount to treat or prevent a pathogenic infection (e.g., one or more pathogenic infections). The terms “treat” or “treatment” refer to reducing or alleviating one or more of the symptoms associated with a pathogenic infection, reducing the amount of bacterial toxin produced by the pathogenic infection, and/or reducing the bacterial load of the pathogenic infection. The terms “prevent” or “prevention” encompass prophylactic administration and may reduce the incidence or likelihood of pathogenic infection or a recurrent or chronic pathogenic infection. For instance, in some embodiments, administration of the compositions provided herein result in a healthy microbiome that is refractory to pathogenic infection, thereby preventing the pathogenic infection.

As used herein, a “therapeutically effective amount” of a composition, such as a pharmaceutical composition, is any amount that results in a desired response or outcome in a subject, such as those described herein, including but not limited to prevention of infection, an immune response or an enhanced immune response to the pathogenic infection, prevention or reduction of symptoms associated with pathogenic infection, and/or a reduction or inhibition of toxin production by the pathogenic infection. It should be appreciated that the term “effective amount” may be expressed in number of bacteria or bacterial spores to be administered. It should further be appreciated that the bacteria can multiply once administered. Thus, administration of even a relatively small amount of bacteria may have therapeutic effects.

In some embodiments, the therapeutically effective amount of any of the compositions described herein is an amount sufficient to enhance survival of the subject, reduce the bacterial burden of the pathogenic infection in the subject, and/or reduce or inhibit toxin production by the pathogenic infection. In some embodiments, the therapeutically effective amount is an amount sufficient to reduce the bacterial burden of the pathogenic infection in a fecal sample from the subject by at least 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 1000-fold, 104-fold, 105-fold or more, as compared to the bacterial burden in a subject with a pathogenic infection that has not received any of the compositions described herein, or as compared to a fecal sample from the same subject that was collected prior to administration of any of the compositions.

In some embodiments, the compositions provided herein inhibit the production of a bacterial toxin, e.g., C. difficile Toxin B. In some embodiments, the therapeutically effective amount is an amount sufficient to reduce or inhibit the amount of bacterial toxin (e.g., C. difficile Toxin B) produced by pathogenic infection in a fecal sample from the subject by at least 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 150-fold, 200-fold, 500-fold or more, as compared to the amount of the bacterial toxin in a subject with a pathogenic infection that has not received any of the compositions described herein or as compared to a fecal sample from the same subject that was collected prior to administration of any of the compositions.

In some embodiments, the therapeutically effective amount is an amount sufficient to recolonize or repopulate the gastrointestinal tract of the subject with non-pathogenic bacteria. In some embodiments, the therapeutically effective amount is an amount sufficient to graft one or more of the bacterial strains of the composition in the gastrointestinal tract of the subject. In some embodiments, a fecal sample is obtained from the subject to assess the bacterial burden of the pathogenic infection and/or evaluate the efficacy of administration of the bacterial compositions described herein. In some embodiments, the microbiota of the subject (e.g., the identity and abundance of strains and/or species of the microbiota) may be assessed to determine a disease state of the subject and/or assess progress of the treatment. In some embodiments, the microbiota of the subject having a pathogenic infection is compared to the microbiota of a healthy subject, such as a subject that is not experiencing or has not experienced the pathogenic infection. In some embodiments, the microbiota of the subject having a pathogenic infection is compared to the microbiota of the same subject from a fecal sample obtained from the subject prior to the pathogenic infection.

Any of the compositions described herein, including the pharmaceutical compositions and food products comprising the compositions (see below), may contain bacterial strains in any form, for example in an aqueous form, such as a solution or a suspension, embedded in a semi-solid form, in a powdered form or freeze dried form. In some embodiments, the composition or the bacterial strains of the composition are lyophilized. In some embodiments, a subset of the bacterial strains in a composition is lyophilized. Methods of lyophilizing compositions, specifically compositions comprising bacteria, are well known in the art. See, e.g., U.S. Pat. Nos. 3,261,761, 4,205,132, WO 2014/029578, and WO 2012/098358, incorporated herein by reference in their entireties. The bacteria may be lyophilized as a combination and/or the bacteria may be lyophilized separately and combined prior to administration. A bacterial strain may be combined with a pharmaceutical excipient prior to combining it with the other bacterial strain or multiple lyophilized bacteria may be combined while in lyophilized form and the mixture of bacteria, once combined may subsequently be combined with a pharmaceutical excipient. In some embodiments, the bacterial strain is a lyophilized cake. In some embodiments, the compositions comprising the one or more bacterial strains are a lyophilized cake.

The bacterial strains of the composition can be manufactured using fermentation techniques well known in the art. In some embodiments, the active ingredients are manufactured using anaerobic fermenters, which can support the rapid growth of anaerobic bacterial species. The anaerobic fermenters may be, for example, stirred tank reactors or disposable wave bioreactors. Culture media such as BL media and EG media, or similar versions of these media devoid of animal components, can be used to support the growth of the bacterial species. The bacterial product can be purified and concentrated from the fermentation broth by traditional techniques, such as centrifugation and filtration, and can optionally be dried and lyophilized by techniques well known in the art.

In some embodiments, the composition of bacterial strains may be formulated for administration as a pharmaceutical composition. The term “pharmaceutical composition” as used herein means a product that results from the mixing or combining of at least one active ingredient, such as any two or more purified bacterial strains described herein, and one or more inactive ingredients, which may include one or more pharmaceutically acceptable excipient.

An “acceptable” excipient refers to an excipient that must be compatible with the active ingredient and not deleterious to the subject to which it is administered. In some embodiments, the pharmaceutically acceptable excipient is selected based on the intended route of administration of the composition, for example a composition for oral or nasal administration may comprise a different pharmaceutically acceptable excipient than a composition for rectal administration. Examples of excipients include sterile water, physiological saline, solvent, a base material, an emulsifier, a suspending agent, a surfactant, a stabilizer, a flavoring agent, an aromatic, an excipient, a vehicle, a preservative, a binder, a diluent, a tonicity adjusting agent, a soothing agent, a bulking agent, a disintegrating agent, a buffer agent, a coating agent, a lubricant, a colorant, a sweetener, a thickening agent, and a solubilizer.

Pharmaceutical compositions of the invention can be prepared in accordance with methods well known and routinely practiced in the art (see e.g., Remington: The Science and Practice of Pharmacy, Mack Publishing Co. 20th ed. 2000). The pharmaceutical compositions described herein may further comprise any carriers or stabilizers in the form of a lyophilized formulation or an aqueous solution. Acceptable excipients, carriers, or stabilizers may include, for example, buffers, antioxidants, preservatives, polymers, chelating reagents, and/or surfactants. Pharmaceutical compositions are preferably manufactured under GMP conditions. The pharmaceutical compositions can be used orally, nasally or parenterally, for instance, in the form of capsules, tablets, pills, sachets, liquids, powders, granules, fine granules, film-coated preparations, pellets, troches, sublingual preparations, chewables, buccal preparations, pastes, syrups, suspensions, elixirs, emulsions, liniments, ointments, plasters, cataplasms, transdermal absorption systems, lotions, inhalations, aerosols, injections, suppositories, and the like.

In some embodiments, the bacteria are formulated for delivery to the intestines (e.g., the small intestine and/or the colon). In some embodiments, the bacteria are formulated with an enteric coating that increases the survival of the bacteria through the harsh environment in the stomach. The enteric coating is one which resists the action of gastric juices in the stomach so that the bacteria which are incorporated therein will pass through the stomach and into the intestines. The enteric coating may readily dissolve when in contact with intestinal fluids, so that the bacteria enclosed in the coating will be released in the intestinal tract. Enteric coatings may consist of polymer and copolymers well known in the art, such as commercially available EUDRAGIT (Evonik Industries). (See e.g., Zhang, AAPS Pharm Sci Tech, (2016) 17 (1), 56-67).

The bacteria may also be formulated for rectal delivery to the intestine (e.g., the colon). Thus, in some embodiments, the bacterial compositions may be formulated for delivery by suppository, colonoscopy, endoscopy, sigmoidoscopy or enema. A pharmaceutical preparation or formulation and particularly a pharmaceutical preparation for oral administration, may include an additional component that enables efficient delivery of the compositions of the disclosure to the intestine (e.g., the colon). A variety of pharmaceutical preparations that allow for the delivery of the compositions to the intestine (e.g., the colon) can be used. Examples thereof include pH sensitive compositions, more specifically, buffered sachet formulations or enteric polymers that release their contents when the pH becomes alkaline after the enteric polymers pass through the stomach. When a pH sensitive composition is used for formulating the pharmaceutical preparation, the pH sensitive composition is preferably a polymer whose pH threshold of the decomposition of the composition is between about 6.8 and about 7.5. Such a numeric value range is a range in which the pH shifts toward the alkaline side at a distal portion of the stomach, and hence is a suitable range for use in the delivery to the colon. It should further be appreciated that each part of the intestine (e.g., the duodenum, jejunum, ileum, cecum, colon, and rectum) has a different biochemical and chemical environment. For instance, parts of the intestines have different pHs, allowing for targeted delivery by compositions that have a specific pH sensitivity. Thus, the compositions provided herein may be formulated for delivery to the intestine or specific parts of the intestine (e.g., the duodenum, jejunum, ileum, cecum, colon, and rectum) by providing formulations with the appropriate pH sensitivity. (See e.g., Villena et al., Int J Pharm 2015, 487 (1-2): 314-9).

Another embodiment of a pharmaceutical preparation useful for delivery of the compositions to the intestine (e.g., the colon) is one that ensures the delivery to the colon by delaying the release of the contents (e.g., the bacterial strains) by approximately 3 to 5 hours, which corresponds to the small intestinal transit time. In one embodiment of a pharmaceutical preparation for delayed release, a hydrogel is used as a shell. The hydrogel is hydrated and swells upon contact with gastrointestinal fluid, with the result that the contents are effectively released (released predominantly in the colon). Delayed release dosage units include drug-containing compositions having a material which coats or selectively coats a drug or active ingredient to be administered. Examples of such a selective coating material include in vivo degradable polymers, gradually hydrolyzable polymers, gradually water-soluble polymers, and/or enzyme degradable polymers. A wide variety of coating materials for efficiently delaying the release is available and includes, for example, cellulose-based polymers such as hydroxypropyl cellulose, acrylic acid polymers and copolymers such as methacrylic acid polymers and copolymers, and vinyl polymers and copolymers such as polyvinylpyrrolidone.

Additional examples of pharmaceutical compositions that allow for the delivery to the intestine (e.g., the colon) include bioadhesive compositions which specifically adhere to the colonic mucosal membrane (for example, a polymer described in the specification of U.S. Pat. No. 6,368,586) and compositions into which a protease inhibitor is incorporated for protecting particularly a biopharmaceutical preparation in the gastrointestinal tracts from decomposition due to an activity of a protease.

Another example of a system enabling the delivery to the intestine (e.g., the colon) is a system of delivering a composition to the colon by pressure change in such a way that the contents are released by utilizing pressure change caused by generation of gas in bacterial fermentation at a distal portion of the stomach. Such a system is not particularly limited, and a more specific example thereof is a capsule which has contents dispersed in a suppository base and which is coated with a hydrophobic polymer (for example, ethyl cellulose).

A further example of a system enabling the delivery of a composition to the intestine (e.g., the colon), is a composition that includes a coating that can be removed by an enzyme present in the gut (e.g., the colon), such as, for example, a carbohydrate hydrolase or a carbohydrate reductase. Such a system is not particularly limited, and more specific examples thereof include systems which use food components such as non-starch polysaccharides, amylose, xanthan gum, and azopolymers. The compositions provided herein can also be delivered to specific target areas, such as the intestine, by delivery through an orifice (e.g., a nasal tube) or through surgery. In addition, the compositions provided herein that are formulated for delivery to a specific area (e.g., the cecum or the colon), may be administered by a tube (e.g., directly into the small intestine). Combining mechanical delivery methods such as tubes with chemical delivery methods such as pH specific coatings, allow for the delivery of the compositions provided herein to a desired target area (e.g., the cecum or the colon).

The compositions comprising bacterial strains can be formulated into pharmaceutically acceptable dosage forms by conventional methods known to those of skill in the art. Dosage regimens are adjusted to provide the optimum desired response (e.g., the prophylactic or therapeutic effect). In some embodiments, the dosage form of the composition is a tablet, pill, capsule, powder, granules, solution, or suppository. In some embodiments, the pharmaceutical composition is formulated for oral administration. In some embodiments, the pharmaceutical composition is formulated such that the bacteria of the composition, or a portion thereof, remain viable after passage through the stomach of the subject. In some embodiments, the pharmaceutical composition is formulated for rectal administration. e.g. as a suppository. In some embodiments, the pharmaceutical composition is formulated for delivery to the intestine or a specific area of the intestine (e.g., the colon) by providing an appropriate coating (e.g., a pH specific coating, a coating that can be degraded by target area specific enzymes, or a coating that can bind to receptors that are present in a target area).

Dosages of the active ingredients in the pharmaceutical compositions of the present invention can be varied so as to obtain an amount of the active ingredient which is effective to achieve the desired pharmaceutical response for a particular subject, composition, and mode of administration, without being toxic or having an adverse effect on the subject. The selected dosage level depends upon a variety of factors including the activity of the particular compositions of the present invention employed, the route of administration, the time of administration, the duration of the treatment, other drugs, compounds and/or materials used in combination with the particular compositions employed, the age, sex, weight, condition, general health and prior medical history of the subject being treated, and like factors.

A physician, veterinarian or other trained practitioner, can start doses of the pharmaceutical composition at levels lower than that required to achieve the desired therapeutic effect and gradually increase the dosage until the desired effect (e.g., treatment of a pathogenic infection, reduction of bacterial burden of pathogenic infection, reduction or inhibition of toxin production) is achieved. In general, effective doses of the compositions of the present invention, for the prophylactic treatment of groups of people as described herein vary depending upon many different factors, including routes of administration, physiological state of the subject, whether the subject is human or an animal, other medications administered, and the therapeutic effect desired. In some embodiments, the dosing regimen entails oral administration of a dose of any of the compositions described herein. In some embodiments, the dosing regimen entails oral administration of multiple doses of any of the compositions described herein. In some embodiments, the composition is administered orally to the subject once, twice, 3 times, 4 times, 5 times, 6 times, 7 times, 8 times, 9 times, or at least 10 times.

The compositions, including the pharmaceutical compositions disclosed herein, can include a range of active ingredients (e.g., live bacteria, bacteria in spore form). The amount of bacteria in the compositions may be expressed in weight, number of bacteria and/or CFUs (colony forming units).

In some embodiments, the compositions disclosed herein contain about 10, about 10², about 10³, about 10⁴, about 10⁵, about 10⁶, about 10⁷, about 10⁸, about 10⁹, about 10¹⁰, about 10¹¹, about 10¹², about 10¹³ or more of each of the bacteria of the composition per dosage amount. In some embodiments, the compositions disclosed herein contain about 10, about 10², about 10³, about 10⁴, about 10⁵, about 10⁶, about 10⁷, about 10⁸, about 10⁹, about 10¹⁰, about 10¹¹, about 10¹², about 10¹³ or more total bacteria per dosage amount. It should further be appreciated that the bacteria of the compositions may be present in different amounts. Thus, for instance, as a non-limiting example, a composition may include 10³ of bacteria A, 10⁴ of bacteria B and 10⁶ of bacteria C. In some embodiments, the compositions disclosed herein contain about 10, about 10², about 10³, about 10⁴, about 10⁵, about 10⁶, about 10⁷, about 10⁸, about 10⁹, about 10¹⁰, about 10¹¹, about 10¹², about 10¹³ or more CFUs of each of the bacteria in the composition per dosage amount. In some embodiments, the compositions disclosed herein contain about 10¹, about 10², about 10³, about 10⁴, about 10⁵, about 10⁶, about 10⁷, about 10⁸, about 10⁹, about 10¹⁰, about 10¹¹, about 10¹², about 10¹³ or more CFUs in total for all of the bacteria combined per dosage amount. As discussed above, bacteria of the compositions may be present in different amounts. In some embodiments, the compositions disclosed herein contain about 10⁻⁷, about 10⁻⁶, about 10⁻⁵, about 10⁻⁴, about 10⁻³, about 10⁻², about 10⁻¹ or more grams of each of the bacteria in the composition per dosage amount. In some embodiments, the compositions disclosed herein contain about 10⁻⁷, about 10⁻⁶, about 10⁻⁵, about 10⁻⁴, about 10⁻³, about 10⁻², about 10⁻¹ or more grams in total for all of the bacteria combined per dosage amount. In some embodiment, the dosage amount is one administration device (e.g., one table, pill or capsule). In some embodiment, the dosage amount is the amount that is administered in a particular period (e.g., one day or one week).

In some embodiments, the compositions disclosed herein contain between 10 and 10¹³, between 10² and 10¹³, between 10³ and 10¹³, between 10⁴ and 10¹³, between 10⁵ and 10¹³, between 10⁶ and 10¹³, between 10⁷ and 10¹³, between 10⁸ and 10¹³, between 10⁹ and 10¹³, between 10¹⁰ and 10¹³, between 10¹¹ and 10¹³, between 10¹² and 10¹³, between 10 and 10¹², between 10² and 10¹², between 10³ and 10¹², between 10⁴ and 10¹², between 10⁵ and 10¹², between 10⁶ and 10¹², between 10⁷ and 10¹², between 10⁸ and 10¹², between 10⁹ and 10¹², between 10¹⁰ and 10¹², between 10¹¹ and 10¹², between 10 and 10¹¹, between 10² and 10¹¹, between 10³ and 10¹¹, between 10⁴ and 10¹¹, between 10⁵ and 10¹¹, between 10⁶ and 10¹¹, between 10⁷ and 10¹¹, between 10⁸ and 10¹¹, between 10⁹ and 10¹¹, between 10¹⁰ and 10¹¹, between 10 and 10¹⁰, between 10² and 10¹⁰, between 10³ and 10¹⁰, between 10⁴ and 10¹⁰, between 10⁵ and 10¹⁰, between 10⁶ and 10¹⁰, between 10⁷ and 10¹⁰, between 10⁸ and 10¹⁰, between 10⁹ and 10¹⁰, between 10 and 10⁹, between 10² and 10⁹, between 10³ and 10⁹, between 10⁴ and 10⁹, between 10⁵ and 10⁹, between 10⁶ and 10⁹, between 10⁷ and 10⁹, between 10⁸ and 10⁹, between 10 and 10⁸, between 10² and 10⁸, between 10³ and 10⁸, between 10⁴ and 10⁸, between 10⁵ and 10⁸, between 10⁶ and 10⁸, between 10⁷ and 10⁸, between 10 and 10⁷, between 10² and 10⁷, between 10³ and 10⁷, between 10⁴ and 10⁷, between 10⁵ and 10⁷, between 10⁶ and 10⁷, between 10 and 10⁶, between 10² and 10⁶, between 10³ and 10⁶, between 10⁴ and 10⁶, between 10⁵ and 10⁶, between 10 and 10⁵, between 10² and 10⁵, between 10³ and 10⁵, between 10⁴ and 10⁵, between 10 and 10⁴, between 10² and 10⁴, between 10³ and 10⁴, between 10 and 10³, between 10² and 10³, or between 10 and 10² of each of the bacteria of the composition per dosage amount.

In some embodiments, the compositions disclosed herein contain between 10 and 10¹³, between 10² and 10¹³, between 10³ and 10¹³, between 10⁴ and 10¹³, between 10⁵ and 10¹³, between 10⁶ and 10¹³, between 10⁷ and 10¹³, between 10⁸ and 10¹³, between 10⁹ and 10¹³, between 10¹⁰ and 10¹³, between 10¹¹ and 10¹³, between 10¹² and 10¹³, between 10 and 10¹², between 10² and 10¹², between 10³ and 10¹², between 10⁴ and 10¹², between 10⁵ and 10¹², between 10⁶ and 10¹², between 10⁷ and 10¹², between 10⁸ and 10¹², between 10⁹ and 10¹², between 10¹⁰ and 10¹², between 10¹¹ and 10¹², between 10 and 10¹¹, between 10² and 10¹¹, between 10³ and 10¹¹, between 10⁴ and 10¹¹, between 10⁵ and 10¹¹, between 10⁶ and 10¹¹, between 10⁷ and 10¹¹, between 10⁸ and 10¹¹, between 10⁹ and 10¹¹, between 10¹⁰ and 10¹¹, between 10 and 10¹⁰, between 10² and 10¹⁰, between 10³ and 10¹⁰, between 10⁴ and 10¹⁰, between 10⁵ and 10¹⁰, between 10⁶ and 10¹⁰, between 10⁷ and 10¹⁰, between 10⁸ and 10¹⁰, between 10⁹ and 10¹⁰, between 10 and 10⁹, between 10² and 10⁹, between 10³ and 10⁹, between 10⁴ and 10⁹, between 10⁵ and 10⁹, between 10⁶ and 10⁹, between 10⁷ and 10⁹, between 10⁸ and 10⁹, between 10 and 10⁸, between 10² and 10⁸, between 10³ and 10⁸, between 10⁴ and 10⁸, between 10⁵ and 10⁸, between 10⁶ and 10⁸, between 10⁷ and 10⁸, between 10 and 10⁷, between 10² and 10⁷, between 10³ and 10⁷, between 10⁴ and 10⁷, between 10⁵ and 10⁷, between 10⁶ and 10⁷, between 10 and 10⁶, between 10² and 10⁶, between 10³ and 10⁶, between 10⁴ and 10⁶, between 10⁵ and 10⁶, between 10 and 10⁵, between 10² and 10⁵, between 10³ and 10⁵, between 10⁴ and 10⁵, between 10 and 10⁴, between 10² and 10⁴, between 10³ and 10⁴, between 10 and 10³, between 10² and 10³, or between 10 and 10² total bacteria per dosage amount.

In some embodiments, the compositions disclosed herein contain between 10 and 10¹³, between 10² and 10¹³, between 10³ and 10¹³, between 10⁴ and 10¹³, between 10⁵ and 10¹³, between 10⁶ and 10¹³, between 10⁷ and 10¹³, between 10⁸ and 10¹³, between 10⁹ and 10¹³, between 10¹⁰ and 10¹³, between 10¹¹ and 10¹³, between 10¹² and 10¹³, between 10 and 10¹², between 10² and 10¹², between 10³ and 10¹², between 10⁴ and 10¹², between 10⁵ and 10¹², between 10⁶ and 10¹², between 10⁷ and 10¹², between 10⁸ and 10¹², between 10⁹ and 10¹², between 10¹⁰ and 10¹², between 10¹¹ and 10¹², between 10 and 10¹¹, between 10² and 10¹¹, between 10³ and 10¹¹, between 10⁴ and 10¹¹, between 10⁵ and 10¹¹, between 10⁶ and 10¹¹, between 10⁷ and 10¹¹, between 10⁸ and 10¹¹, between 10⁹ and 10¹¹, between 10¹⁰ and 10¹¹, between 10 and 10¹⁰, between 10² and 10¹⁰, between 10³ and 10¹⁰, between 10⁴ and 10¹⁰, between 10⁵ and 10¹⁰, between 10⁶ and 10¹⁰, between 10⁷ and 10¹⁰, between 10⁸ and 10¹⁰, between 10⁹ and 10¹⁰, between 10 and 10⁹, between 10² and 10⁹, between 10³ and 10⁹, between 10⁴ and 10⁹, between 10⁵ and 10⁹, between 10⁶ and 10⁹, between 10⁷ and 10⁹, between 10⁸ and 10⁹, between 10 and 10⁸, between 10² and 10⁸, between 10³ and 10⁸, between 10⁴ and 10⁸, between 10⁵ and 10⁸, between 10⁶ and 10⁸, between 10⁷ and 10⁸, between 10 and 10⁷, between 10² and 10⁷, between 10³ and 10⁷, between 10⁴ and 10⁷, between 10⁵ and 10⁷, between 10⁶ and 10⁷ between 10 and 10⁶, between 10² and 10⁶, between 10³ and 10⁶, between 10⁴ and 10⁶, between 10⁵ and 10⁶, between 10 and 10⁵, between 10² and 10⁵, between 10³ and 10⁵, between 10⁴ and 10⁵, between 10 and 10⁴, between 10² and 10⁴, between 10³ and 10⁴, between 10 and 10³, between 10² and 10³, or between 10 and 10² CFUs of each of the bacteria of the composition per dosage amount.

In some embodiments, the compositions disclosed herein contain between 10 and 10¹³, between 10² and 10¹³, between 10³ and 10¹³, between 10⁴ and 10¹³, between 10⁵ and 10¹³, between 10⁶ and 10¹³, between 10⁷ and 10¹³, between 10⁸ and 10¹³, between 10⁹ and 10¹³, between 10¹⁰ and 10¹³, between 10¹¹ and 10¹³, between 10¹² and 10¹³, between 10 and 10¹², between 10² and 10¹², between 10³ and 10¹², between 10⁴ and 10¹², between 10⁵ and 10¹², between 10⁶ and 10¹², between 10⁷ and 10¹², between 10⁸ and 10¹², between 10⁹ and 10¹², between 10¹⁰ and 10¹², between 10¹¹ and 10¹², between 10 and 10¹¹, between 10² and 10¹¹, between 10³ and 10¹¹, between 10⁴ and 10¹¹, between 10⁵ and 10¹¹, between 10⁶ and 10¹¹, between 10⁷ and 10¹¹, between 10⁸ and 10¹¹, between 10⁹ and 10¹¹, between 10¹⁰ and 10¹¹, between 10 and 10¹⁰, between 10² and 10¹⁰, between 10³ and 10¹⁰, between 10⁴ and 10¹⁰, between 10⁵ and 10¹⁰, between 10⁶ and 10¹⁰, between 10⁷ and 10¹⁰, between 10⁸ and 10¹⁰, between 10⁹ and 10¹⁰, between 10 and 10⁹, between 10² and 10⁹, between 10³ and 10⁹, between 10⁴ and 10⁹, between 10⁵ and 10⁹, between 10⁶ and 10⁹, between 10⁷ and 10⁹, between 10⁸ and 10⁹, between 10 and 10⁸, between 10² and 10⁸, between 10³ and 10⁸, between 10⁴ and 10⁸, between 10⁵ and 10⁸, between 10⁶ and 10⁸, between 10⁷ and 10⁸, between 10 and 10⁷, between 10² and 10⁷, between 10³ and 10⁷, between 10⁴ and 10⁷, between 10⁵ and 10⁷, between 10⁶ and 10⁷, between 10 and 10⁶, between 10² and 10⁶, between 10³ and 10⁶, between 10⁴ and 10⁶, between 10⁵ and 10⁶, between 10 and 10⁵, between 10² and 10⁵, between 10³ and 10⁵, between 10⁴ and 10⁵, between 10 and 10⁴, between 10² and 10⁴, between 10³ and 10⁴, between 10 and 10³, between 10² and 10³, or between 10 and 10² total CFUs per dosage amount.

In some embodiments, the compositions disclosed herein contain between 10⁻⁷ and 10¹, between 10⁻⁶ and 10¹, between 10⁻⁵ and 10¹, between 10⁻⁴ and 10¹, between 10⁻³ and 10¹, between 10⁻² and 10¹, between 10⁻⁷ and 10⁻², between 10⁻⁶ and 10⁻², between 10⁻⁵ and 10⁻², between 10⁻⁴ and 10⁻², between 10⁻³ and 10⁻², between 10⁻⁷ and 10⁻³, between 10⁻⁶ and 10⁻³, between 10⁵ and 10⁻³, between 10⁻⁴ and 10⁻³, between 10⁻⁷ and 10⁻⁴, between 10⁻⁶ and 10⁻⁴, between 10⁻⁵ and 10⁻⁴, between 10⁻⁷ and 10⁻⁵, between 10⁻⁶ and 10⁻⁵, or between 10⁻⁷ and 10⁻⁶ grams of each of the bacteria in the composition per dosage amount.

In some embodiments, the compositions disclosed herein contain between 10⁻⁷ and 10⁻¹, between 10⁻⁶ and 10⁻¹, between 10⁻⁵ and 10⁻¹, between 10⁻⁴ and 10⁻¹, between 10⁻³ and 10⁻¹, between 10⁻² and 10⁻¹, between 10⁻⁷ and 10⁻², between 10⁻⁶ and 10⁻², between 10⁵ and 10⁻², between 10⁻⁴ and 10⁻², between 10⁻³ and 10⁻², between 10⁻⁷ and 10⁻³, between 10⁻⁶ and 10⁻³, between 10⁵ and 10⁻³, between 10⁻⁴ and 10⁻³, between 10⁻⁷ and 10⁻⁴, between 10⁻⁶ and 10⁻⁴, between 10⁻⁵ and 10⁻⁴, between 10⁻⁷ and 10⁻⁵, between 10⁻⁶ and 10⁻⁵, or between 10⁻⁷ and 10⁻⁶ grams of all of the bacteria combined per dosage amount.

Also with the scope of the present disclosure are food products comprising any of the bacterial strains described herein and a nutrient. Food products are, in general, intended for the consumption of a human or an animal. Any of the bacterial strains described herein may be formulated as a food product. In some embodiments, the bacterial strains are formulated as a food product in spore form. In some embodiments, the bacterial strains are formulated as a food product in vegetative form. In some embodiments, the food product comprises both vegetative bacteria and bacteria in spore form. The compositions disclosed herein can be used in a food or beverage, such as a health food or beverage, a food or beverage for infants, a food or beverage for pregnant women, athletes, senior citizens or other specified group, a functional food, a beverage, a food or beverage for specified health use, a dietary supplement, a food or beverage for patients, or an animal feed. Non-limiting examples of the foods and beverages include various beverages such as juices, refreshing beverages, tea beverages, drink preparations, jelly beverages, and functional beverages; alcoholic beverages such as beers; carbohydrate-containing foods such as rice food products, noodles, breads, and pastas; paste products such as fish hams, sausages, paste products of seafood; retort pouch products such as curries, food dressed with a thick starchy sauces, soups; dairy products such as milk, dairy beverages, ice creams, cheeses, and yogurts; fermented products such as fermented soybean pastes, yogurts, fermented beverages, and pickles; bean products; various confectionery products such as Western confectionery products including biscuits, cookies, and the like, Japanese confectionery products including steamed bean-jam buns, soft adzuki-bean jellies, and the like, candies, chewing gums, gummies, cold desserts including jellies, cream caramels, and frozen desserts; instant foods such as instant soups and instant soy-bean soups; microwavable foods; and the like. Further, the examples also include health foods and beverages prepared in the forms of powders, granules, tablets, capsules, liquids, pastes, and jellies.

Food products containing bacterial strains described herein may be produced using methods known in the art and may contain the amounts of bacteria (e.g., by weight, amount or CFU) as described above. Selection of an appropriate amount of bacteria in the food product may depend on various factors, including for example, the serving size of the food product, the frequency of consumption of the food product, the specific bacterial strains contained in the food product, the amount of water in the food product, and/or additional conditions for survival of the bacteria in the food product.

Examples of food products which may be formulated to contain any of the bacterial strains described herein include, without limitation, a beverage, a drink, a bar, a snack, a dairy product, a confectionery product, a cereal product, a ready-to-eat product, a nutritional formula, such as a nutritional supplementary formulation, a food or beverage additive.

In some embodiments, the subject has not received a dose of an antibiotic prior to administration of the bacterial composition. In some embodiments, the subject has not been administered an antibiotic at least 1, at least 2, at least 3, at least 5, at least 10, at least 15, at least 20, at least 25, at least 30, at least 60, at least 90, at least 120, at least 180 or at least 360 days prior to administration of the compositions provided herein. In some embodiments, the person has not been administered an antibiotic to treat the pathogenic infection. In some embodiments, the compositions provided herein comprise the first treatment of the pathogenic infection.

In some embodiments, the subject may be administered one or more doses of an antibiotic prior to or concurrently with a bacterial composition. Generally, the first line of defense in the treatment of a pathogenic infection is the administration of an antibiotic. In some embodiments, the subject is administered a single dose of an antibiotic prior to the bacterial composition. In some embodiments, the subject is administered multiple doses of an antibiotic prior to the bacterial composition. In some embodiments, the subject is administered at least 2, 3, 4, 5 or more doses of an antibiotic prior to the bacterial composition. In some embodiments, the subject is administered a dose of an antibiotic at substantially the same time as the bacterial composition. Examples of antibiotics that can be administered include, without limitation, kanamycin, gentamicin, colistin, metronidazole, vancomycin, clindamycin, fidaxomicin, and cefoperazone.

The elements and method steps described herein can be used in any combination whether explicitly described or not.

All combinations of method steps as used herein can be performed in any order, unless otherwise specified or clearly implied to the contrary by the context in which the referenced combination is made.

As used herein, the singular forms “a,” “an,” and “the” include plural referents unless the content clearly dictates otherwise.

Numerical ranges as used herein are intended to include every number and subset of numbers contained within that range, whether specifically disclosed or not. Further, these numerical ranges should be construed as providing support for a claim directed to any number or subset of numbers in that range. For example, a disclosure of from 1 to 10 should be construed as supporting a range of from 2 to 8, from 3 to 7, from 5 to 6, from 1 to 9, from 3.6 to 4.6, from 3.5 to 9.9, and so forth.

All patents, patent publications, and peer-reviewed publications (i.e., “references”) cited herein are expressly incorporated by reference to the same extent as if each individual reference were specifically and individually indicated as being incorporated by reference. In case of conflict between the present disclosure and the incorporated references, the present disclosure controls.

It is understood that the invention is not confined to the particular construction and arrangement of parts herein illustrated and described, but embraces such modified forms thereof as come within the scope of the claims.

### EXAMPLES

Elucidating Human Gut Microbiota Interactions that Robustly Inhibit Diverse Clostridioides difficile Strains Across Different Nutrient Landscapes

### Summary

The human gut pathogen Clostridioides difficile displays substantial inter-strain genetic variability and confronts a changeable nutrient landscape in the gut. We examined how human gut microbiota inter-species interactions influence the growth and toxin production of various C. difficile strains across different nutrient environments. Negative interactions influencing C. difficile growth are prevalent in an environment containing a single highly accessible resource and sparse in an environment containing C. difficile-preferred carbohydrates. C. difficile toxin production displays significant community-context dependent variation and does not trend with growth-mediated inter-species interactions. C. difficile strains display differences in interactions with Clostridium scindens and the ability to compete for proline. Further, C. difficile shows substantial differences in transcriptional profiles in co-culture with C. scindens or Clostridium hiranonis. C. difficile exhibits massive alterations in metabolism and other cellular processes in co-culture with C. hiranonis, reflecting their similar metabolic niches. C. hiranonis uniquely inhibits the growth and toxin production of diverse C. difficile strains across different nutrient environments and robustly ameliorates disease severity in mice. In sum, understanding the impact of C. difficile strain variability and nutrient environments on inter-species interactions could help improve the effectiveness of anti-C. difficile strategies.

Aspects of the present examples cite to Sulaiman et al. 2024 (Sulaiman J E, Thompson J, Qian Y, Vivas E I, Diener C, Gibbons S M, Safdar N, Venturelli O S. Elucidating human gut microbiota interactions that robustly inhibit diverse Clostridioides difficile strains across different nutrient landscapes. Nat Commun. 2024 Aug. 28; 15(1):7416), which is incorporated herein by reference in its entirety.

### Introduction

The human gut microbiome exists in a dynamic balance between homeostasis and disruption due to the contrasting evolutionary objectives of the host and the resident gut bacteria. Clostridioides difficile is an opportunistic human gut pathogen that can cause life-threatening damage to the colon. Antibiotics are the first-line treatment for C. difficile infection (CDI). However, they also damage the commensal gut microbiota that provides C. difficile colonization resistance and could cause the recurrence of CDI (rCDI) ¹⁻³. Fecal microbiota transplantation (FMT) has proven to be effective for treating rCDI, but the effect of FMT on a patient can vary due to uncharacterized factors and donor microbiota variability ⁴. FMT can also result in the unintentional transfer of antibiotic-resistant bacteria, including other opportunistic pathogens ^{5,6}. To overcome these limitations, defined communities of commensal bacteria can be designed to inhibit C. difficile. However, low richness communities do not display robustness of anti-C. difficile activity to changes in environmental contexts ^{7,8}. This in turn could contribute to the variability in efficacy in clinical trials of certain living bacterial therapeutics for treating CDI ⁹. We lack an understanding of how environmental context, such as the genetics of C. difficile strains and nutrient environments, impacts the anti-C. difficile activity of human gut communities ^{10,11}.

C. difficile has a diverse population structure comprising hundreds of strain types ¹² that are distributed across at least 8 phylogenetic clades ¹³. This species is defined by a large pangenome ¹⁴, with an ultralow core genome (as low as 16% based on 73 genomes ¹⁵) and extreme levels of evolutionary plasticity that have been molded over long periods through frequent exchange with bacterial gene pools in multiple host environments via horizontal gene transfer ¹⁶⁻¹⁹. This substantial genetic variation among C. difficile strains has downstream impacts on the regulation of metabolic pathways and virulence ^{16,20-22}. For instance, the emergence of the hypervirulent epidemic strain ribotype 027 has been proposed as the major driver of the increase in the prevalence of CDI ^{23,24}. Notably, rCDI is not always due to infection with the same strain, where new strains were observed in 33-56% of recurrent episodes ²⁵⁻²⁹. This suggests that the degree of colonization resistance could vary across different C. difficile strains, potentially leading to differences in patient outcomes.

Interactions with gut microbiota are critical determinants of C. difficile colonization and toxin production, as evidenced by the colonization resistance variability of different microbiome compositions to C. difficile³⁰. Previous studies have elucidated principles that influence C. difficile growth in human gut communities in vitro, such as a strong negative dependence on species richness ³¹, and identified specific mechanisms of C. difficile inhibition. For example, certain species compete with C. difficile for limiting resources, such as the consumption of specific mucus-derived sugars by Akkermansia muciniphila ³² or the utilization of Stickland metabolism amino acids by Clostridium species (e.g. Clostridium bifermentans ^{33,34} and Clostridium scindens ³³). In addition, C. scindens can produce tryptophan-derived antibiotics that inhibit C. difficile growth ³⁵. Clostridium hiranonis was shown to inhibit C. difficile in vitro through more than one mechanism in a single nutrient environment ³¹. However, the contribution of C. difficile strain-level variability to these interactions is currently unknown ^{31,36,37}.

The bottom-up construction of synthetic microbiomes combined with computational modeling ^{38,39} and principled experimental design techniques ⁴⁰ can be used to efficiently navigate large design landscapes of combinations of species. In addition, these bottom-up approaches can provide a deeper understanding of important molecular and ecological mechanisms. For example, a widely used dynamic ecological model referred to as generalized Lotka-Volterra (gLV) can be used to unravel growth-mediated microbial interactions shaping community assembly ⁴¹⁻⁴³. By informing the model with properly collected experimental data, the gLV model can accurately forecast community dynamics as a function of the intrinsic growth of individual species and pairwise interactions with all constituent community members ^{38,44}.

To understand how nutrient and strain-level variability shapes interaction networks with C. difficile, we used a bottom-up approach to build microbial communities combined with computational modeling. We elucidated strain-level differences in inter-species interactions at the transcriptional level using genome-wide transcriptional profiling. In addition, we discovered that the large variation in toxin production of C. difficile in communities was not correlated with growth-mediated inter-species interactions. Our workflow identifies Clostridium hiranonis as a “universal” C. difficile growth and toxin production inhibitor that is robust to variation in strain backgrounds and nutrient environments. This robust inhibition is consistent with its high metabolic niche overlap with C. difficile, which in turn could block the utilization of C. difficile-preferred substrates. Consistent with this notion, genome-wide transcriptional profiling reveals a unique massive alteration of C. difficile metabolism in the presence of C. hiranonis, which is not observed in co-culture with another closely related species, C. scindens. Furthermore, C. hiranonis ameliorated the C. difficile-induced disease severity of mice challenged with two genetically distinct C. difficile strains or subjected to different nutrient perturbations. In sum, we demonstrate that strain-level variability and nutrient environments play an important role in shaping the interactions between C. difficile and human gut communities, and highlight C. hiranonis as a promising candidate to include in the design of robust anti-C. difficile defined consortia.

### Results

C. difficile Strains Display Substantial Phenotypic and Genetic Variability

To understand how the strain-level genetic variability influences C. difficile phenotypes, we characterized 18 C. difficile strains (9 from diseased patients that were diagnosed and treated for CDI and 9 from healthy individuals) and C. difficile DSM 27147 (R20291 reference strain of the epidemic ribotype 027). We individually profiled their growth in a chemically defined media supplemented with carbohydrate sources shown to promote colonization or virulence activities including succinate ^{45,46}, trehalose ^{21,22}, mannitol ^{46,47}, sorbitol ^{46,47}, and various mucus-derived sugars such as sialic acid and n-acetyl-D-glucosamine ^{36,48} (Sulaiman et al. 2024 at FIG. S1a-d, Sulaiman et al. 2024 at Tables S1 and S2). The growth of all C. difficile strains was supported in defined media without any carbohydrate source due to their ability to utilize amino acids through Stickland metabolism. In general, supplementation of glucose, mannitol, n-acetyl-D-glucosamine (GlcNAc), and sialic acid enhanced the growth of all C. difficile strains compared to media without carbohydrate sources.

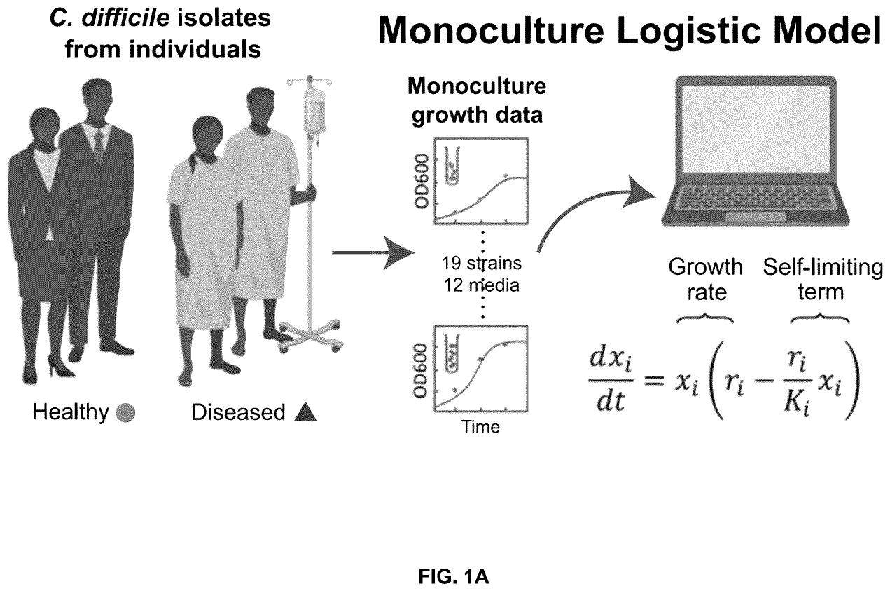

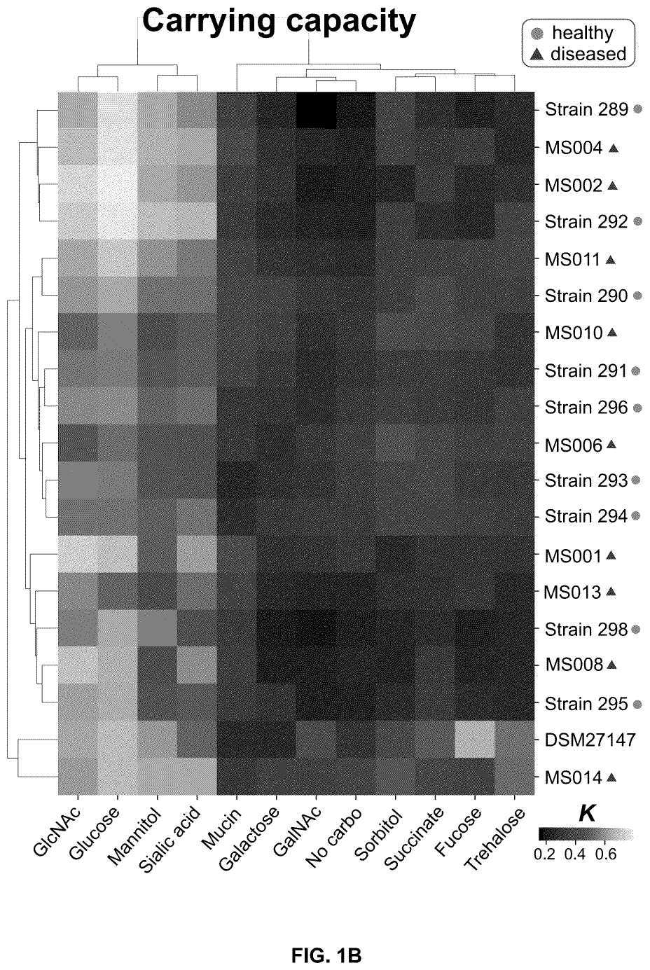

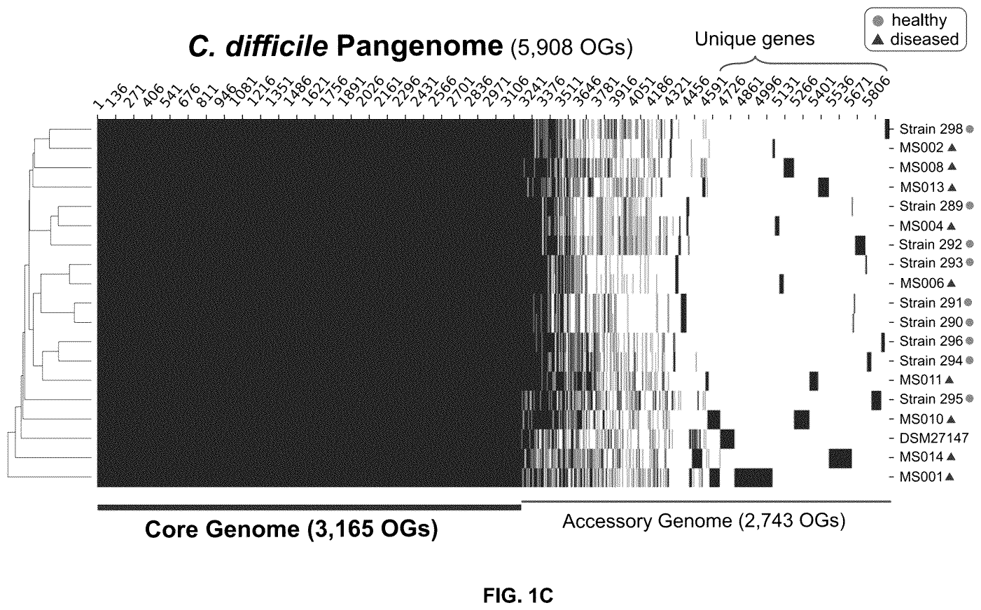

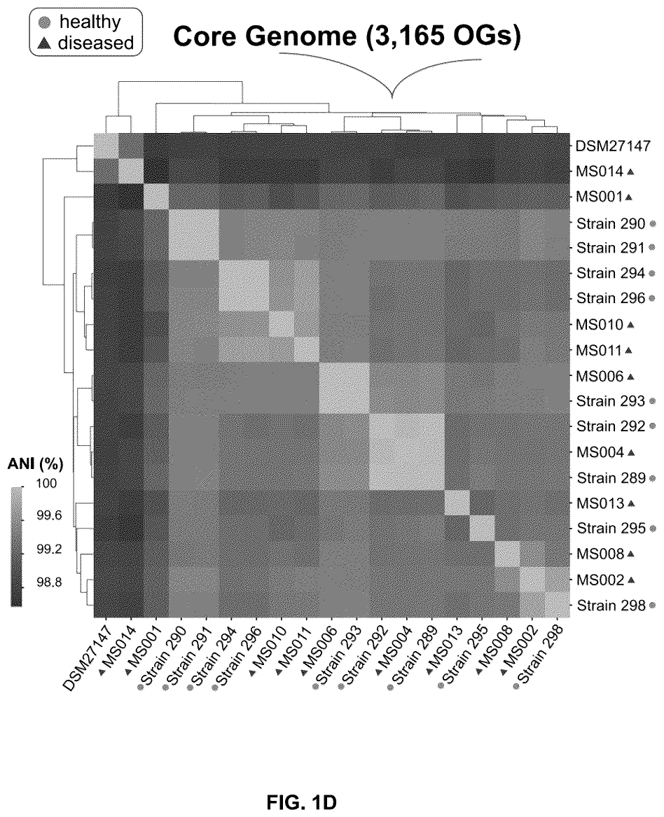

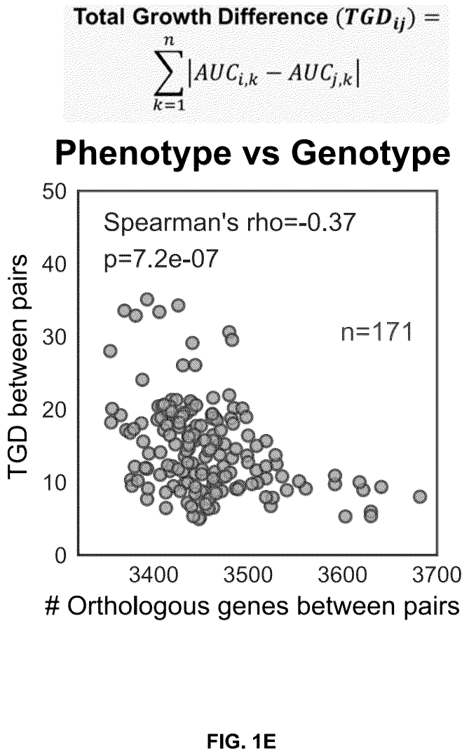

In most single carbohydrate media, C. difficile displayed a unique growth profile that is distinct from the other commensal gut bacteria, where the culture grew rapidly at the beginning followed by a steep decline in OD₆₀₀ during stationary phase at ˜24 h of growth (i.e. non-monotonic growth response). While the variance in monoculture growth biological replicates is low in the first 24 h, this variability increases substantially at the time when OD₆₀₀ declines in stationary phase (Sulaiman et al. 2024 at FIG. S1g-h). This implies that sporulation and cell lysis, in addition to halted cell division as observed by fluorescence microscopy contribute to the observed reduction and variability in OD₆₀₀ (Sulaiman et al. 2024 at FIG. S2). To quantify the variability in growth profiles across C. difficile strains, we fit each growth curve to a logistic model to determine the growth rate (r) and carrying capacity (K) of each strain excluding data points with a >10% reduction in OD₆₀₀ in the late stationary phase (FIGS. 1A-1B, Sulaiman et al. 2024 at FIG. S1e, see Methods). Overall, the logistic model displayed a high goodness of fit to the data (Pearson R=0.98, P<10E-05) (Sulaiman et al. 2024 at FIG. S1f).

We performed whole-genome sequencing on each isolate to provide insights into the genetic variation driving the observed phenotypic variability. The C. difficile genome is comprised of a well-conserved core genome (3,165 orthologous genes) and substantial variation in its accessory genome (FIG. 1C). Metabolic genes varied substantially across the 19 C. difficile strains, where only ˜63% of metabolic genes were shared (Sulaiman et al. 2024 at FIG. S3a). Clustering based on ANI, which represents the Average Nucleotide Identity of all orthologous genes shared between any two genomes, highlighted strains that are more genetically similar to each other (FIG. 1D), such as DSM 27147 and MS014. In addition to other genome similarities, these two strains possessed a mutation in the treR gene (L172I) that confers enhanced trehalose metabolism identified in hypervirulent C. difficile strains²², consistent with their higher capability to utilize trehalose (FIG. 1B). Further, MS001 is clustered separately from the rest of the group based on ANI. MS001 has a much higher number of genes (4110) compared to the other strains (ranging from 3629 to 3892) (Sulaiman et al. 2024 at Table S3), and uniquely lacks the toxins TcdA and TcdB. Indeed, non-toxigenic C. difficile strains have distinct phenotypes compared to toxigenic strains, as a consequence of the variability in their genome 49. In general, there is no pattern between the C. difficile isolates from healthy and sick individuals in terms of their genotype.

To quantify if the genotypic variation displays an informative relationship with phenotypic variation in monoculture, we define the growth difference (GD) as the absolute value of the difference in the AUC of pairs of strains in a specific media. The total growth difference (TGD) is the sum of GD across the 12 media. The TGD and the number of orthologous genes (OGs) or ANI of pairs of C. difficile strains displayed a moderate negative correlation (FIG. 1E, Sulaiman et al. 2024 at FIG. S4a-b). In addition, growth in glucose, trehalose, galactose, and sorbitol was negatively correlated with ANI and the number of OGs (Sulaiman et al. 2024 at FIG. S4c-d). These results suggest that the genotypic variability quantified by these metrics displays an informative relationship with the utilization of certain carbohydrates.

Although the number of genes responsible for most core processes beyond metabolism is similar across isolates, there was large variability in the number of genes related to DNA recombination and integration, which are markers of mobile genetic elements (MGEs) (Sulaiman et al. 2024 at FIG. S3e). This suggests that MGEs play a major role in driving C. difficile genotypic differences, consistent with previous reports ^{50,51}. To characterize the contribution of plasmids to the genome of C. difficile, we searched for high-coverage contigs within genome assemblies and discovered 11 of such instances in 7 of 19 genomes (Sulaiman et al. 2024 at FIG. S5a-c). These putative plasmids contained direct repeats on their termini indicative of being circular. In addition, the putative plasmids do not contain genes that could provide a selective advantage to these strains such as antibiotic resistance or virulence factors (Sulaiman et al. 2024 at Table S5). Interestingly, 4 of the 11 high-coverage contigs map to the same plasmid that is present in four different genetically distant C. difficile isolates from different patients. These isolates also have a highly variable number of conjugative systems and phages, covering 1.4-16.5% of their genomes (Sulaiman et al. 2024 at FIG. S3f, Sulaiman et al. 2024 at Tables S6-S7). In sum, the C. difficile isolates have highly diverse genomes with substantial variability in metabolic genes and mobile genetic elements.

Human Gut Communities Containing Different C. difficile Isolates Display Differences in Interaction Networks

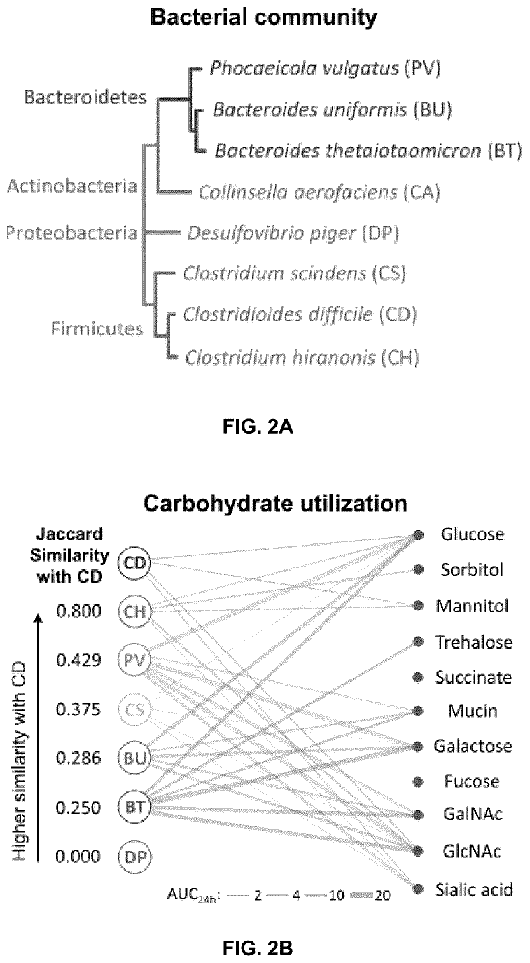

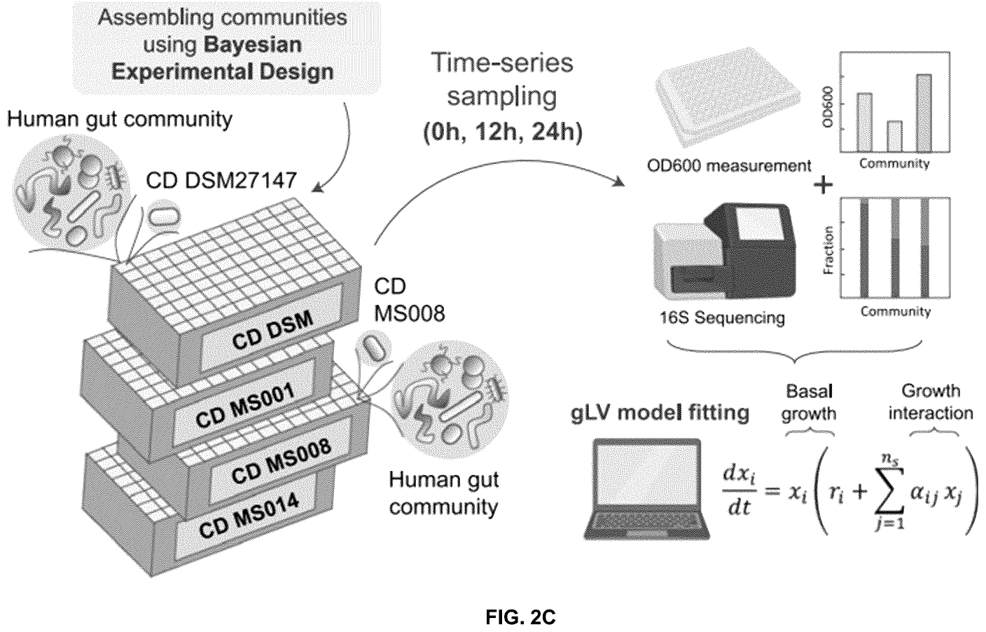

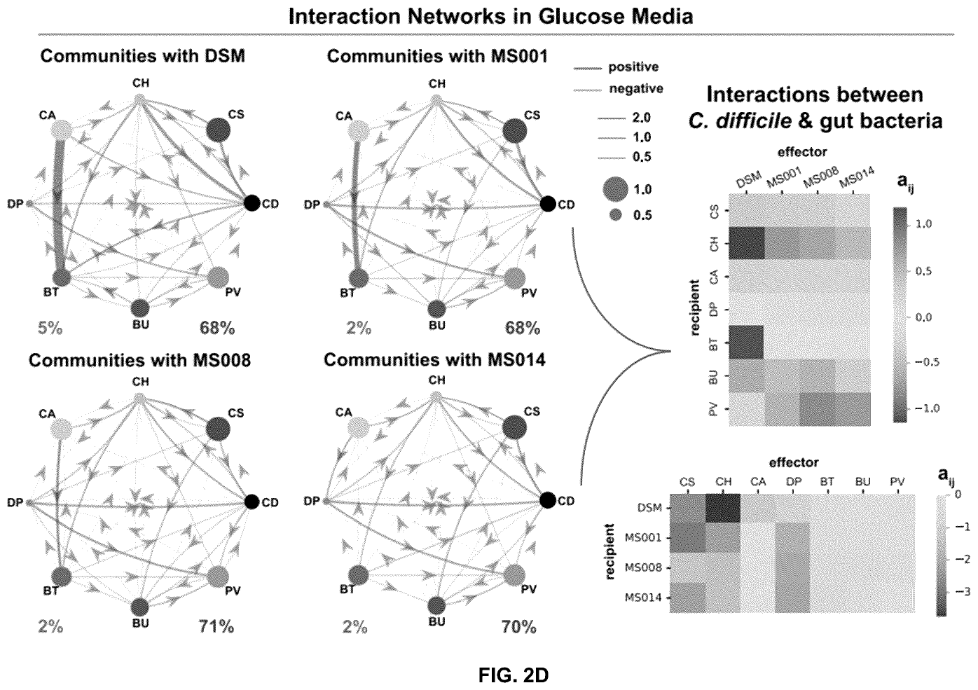

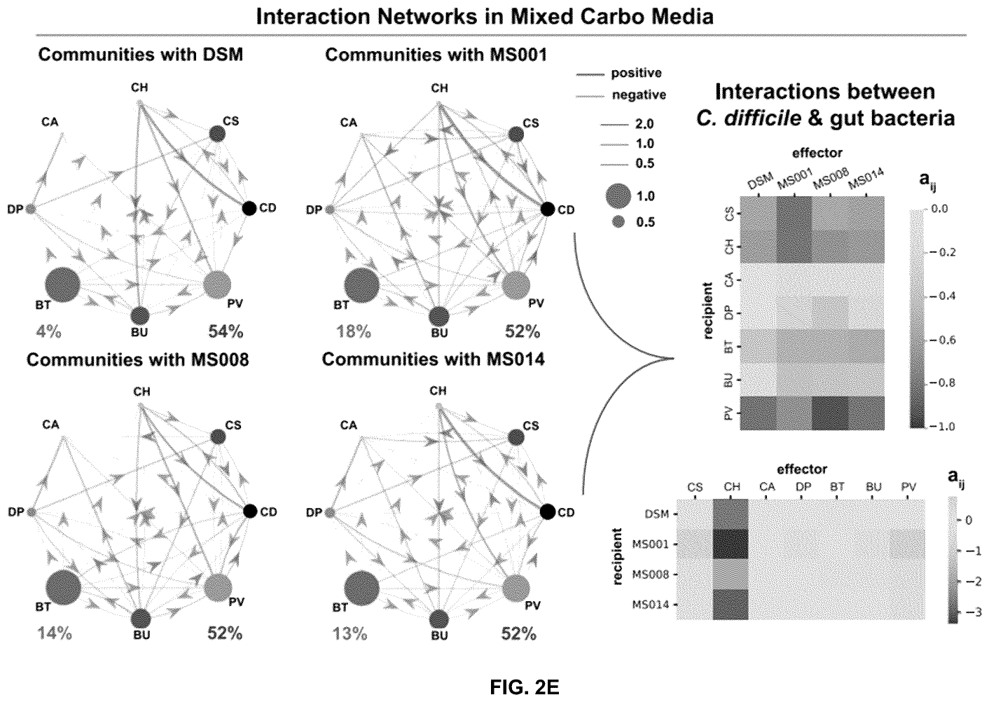

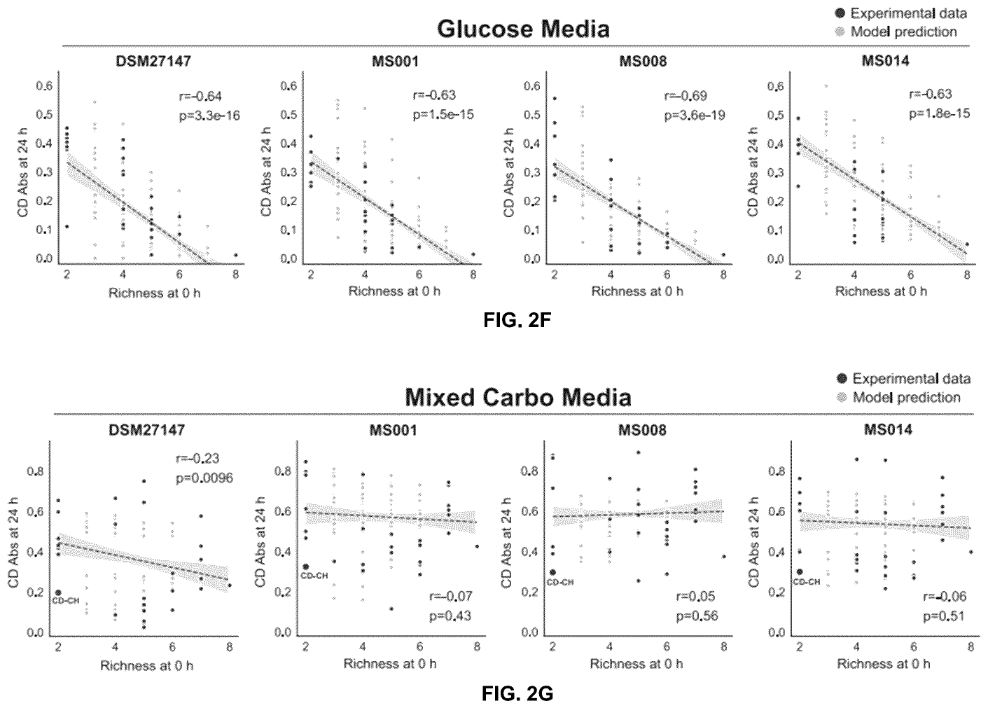

Since human gut microbiota interactions are critical determinants of C. difficile growth and colonization, we investigated how C. difficile genetic variation shapes gut microbiota interspecies interactions. To this end, we built human gut communities from the bottom up with one of 4 diverse C. difficile strains (DSM 27147, MS001, MS008, and MS014) and combinations of 7 gut species (C. scindens (CS), C. hiranonis (CH), Desulfovibrio piger (DP), Bacteroides thetaiotaomicron (BT), Phocaeicola vulgatus (PV) (formerly Bacteroides vulgatus), Bacteroides uniformis (BU), and Collinsella aerofaciens (CA)) (FIG. 2A). Many of these species are prevalent across individuals and span major phyla of the human gut microbiome. These species displayed variation in growth in media with different carbohydrates (Sulaiman et al. 2024 at FIG. S1a-b). The community features CS, previously shown to inhibit the growth of C. difficile in gnotobiotic mice ³⁷, CH which can inhibit C. difficile growth through unknown mechanism ³¹, and Bacteroides species, which have the potential for C. difficile inhibition in different environments ^{36,45,52,53}.

To infer the inter-species interaction networks, we down selected a set of representative C. difficile strains based on their genotypic and phenotypic variations. Strains that have similar genotypes and metabolic genes may display similar interaction networks, whereas interactions may be divergent for strains with large differences in genotype. MS014 shows a similar genotype to DSM 27147 and thus might evolve from the same ancestor, but MS014 was more recently isolated. By contrast, the non-toxigenic strain MS001 has the most different genotype than the other strains, suggesting potentially larger differences in inter-species interactions. Finally, MS008 is genotypically and phenotypically distinct from the other 3 strains (FIG. 1B-1D). In addition, MS008 clustered differently from MS014, DSM 27147 and MS001 based on metabolic genes, suggesting divergent metabolic capabilities (Sulaiman et al. 2024 at FIG. S3a).

Given the key role of resource competition in the ecology of C. difficile 32-34,54, the extent of metabolic niche overlap with C. difficile may be a major variable influencing interactions with human gut bacteria. To quantify the extent of metabolic niche overlap between each gut species and C. difficile, we calculated the Jaccard Similarity of carbohydrate utilization based on the change in growth in the presence and absence of the given carbohydrate (FIG. 2B). Notably, CH displayed the largest metabolic niche overlap of carbohydrate utilization with C. difficile (Jaccard Index=0.8). In addition to the similarities in carbohydrate utilization, CH has been shown to use amino acids via Stickland metabolism, similar to C. difficile ³³.

To study community inter-species interactions in a gut environment with a limited number of resources, we used a defined media containing glucose as the sole carbohydrate source. Glucose can support the growth of most species in monoculture including C. difficile and thus may promote inter-species competition (Sulaiman et al. 2024 at FIG. S6a-c). To quantify the differences in the inter-species interaction networks, we cultured different combinations of species with one of the four C. difficile strains (DSM 27147, MS001, MS008, and MS014) (FIG. 2C). Since there are too many community combinations to be comprehensively explored (635 combinations), we used a Bayesian experimental design approach to select combinations of bacteria that would maximize information content as quantified by the expected Kullback-Leibler divergence between the posterior and prior parameter distributions (see Methods and Sulaiman et al. 2024 at supplementary text section 7 in Supplementary Information) 40. Briefly, a preliminary gLV model was fit to the monoculture growth in glucose media. We used a Bayesian inference approach to approximate the posterior parameter distribution as a multivariate Gaussian. The parameter distribution inferred for the preliminary model was used as a prior to guide the design of 147 combinations of 2 to 8-member sub-communities containing one of the four C. difficile strains (DSM 27147, MS001, MS008, and MS014). Species absolute abundance was determined by multiplying the relative abundance fraction via multiplexed 16S rRNA sequencing by the total biomass obtained by OD₆₀₀ as previously described ^{31,38}. The parameters of the gLV model were inferred based on time-series data of species abundances (0, 12, and 24 h) (Sulaiman et al. 2024 at FIG. S7a, Sulaiman et al. 2024 at DATASET001 in Table S8). Based on the parameter posterior distributions, we analyzed parameters with absolute values that were significantly constrained to be non-zero based on the Wald test ⁵⁵ (Sulaiman et al. 2024 at FIG. S8, Sulaiman et al. 2024 at supplementary text section 5 in Supplementary Information). The Wald test compares the parameter mean to its standard deviation to evaluate whether the peak of the posterior parameter distribution is significantly higher or lower than zero compared to the width of the distribution. The percentage of constrained parameters is 76.6%, 73.4%, 75%, and 75% for communities containing DSM, MS001, MS008, or MS014 respectively. To evaluate model prediction performance on held-out data, we performed 10-fold cross-validation where only community samples were subjected to testing (see Methods). Using a 10-fold cross-validation, the model prediction exhibited good agreement with the measured species abundance in all communities with different C. difficile strains (Pearson's R=0.93-0.95, P<10E-05), demonstrating that our model can capture and predict the trends in species abundance (Sulaiman et al. 2024 at FIG. S7b).

The interaction networks for distinct C. difficile strains displayed a high fraction of negative interactions (68-71%) and C. difficile was inhibited by all species (FIG. 2D). CS and CH display a high magnitude of inhibition towards C. difficile, consistent with their ability to compete for amino acids via Stickland fermentation. Notably, the C. difficile DSM 27147 hypervirulent strain exhibits the largest differences in interaction profile from other C. difficile strains (e.g. interactions with BT, DP, and CH).

In addition to the observed changes in pairwise interactions with C. difficile, other inter-species interactions displayed strain-specific differences. A higher order interaction (HOI) is defined as a substantial change in a pairwise interaction due to the presence of a third community member ^{56,57}. Changes in pairwise interactions due to the presence of different C. difficile strains may suggest HOI. For instance, the interaction coefficients between CA and BT are substantially impacted by the specific C. difficile strain that is present in the community (Sulaiman et al. 2024 at FIG. S7c). To further explore whether C. difficile strain variations could impact CA-BT interactions, we cultured the CA-BT pairwise community in the sterilized spent media of C. difficile (Sulaiman et al. 2024 at FIG. S7d). The abundances of CA and BT in the community were statistically different when cultured in the sterile conditioned media of the different C. difficile strains. This implies that different strains of C. difficile differentially altered the chemical environment, which in turn impacted the interactions between CA and BT. In sum, inferred inter-species interaction networks containing distinct C. difficile strains displayed infrequent direct and indirect differences.

Human Gut Bacteria Infrequently Inhibit C. difficile in the Presence of Preferred Carbohydrates

Antibiotic treatments lead to massive gut bacterial mortality, alternations in the resource landscape, and changes in community composition. This new environment can be exploited by C. difficile ^{45,48,58-61}. To explore community interactions in media that mirrors post-antibiotic environments, we designed a media containing multiple carbohydrates that could be utilized by C. difficile (mixed carbohydrates media) (Sulaiman et al. 2024 at FIG. S9a). In this media, C. difficile strains displayed substantial growth and a diminished decline in OD₆₀₀ in late stationary phase than glucose media (Sulaiman et al. 2024 at FIG. S9b). In pairwise communities, the relative abundance of C. difficile was high in all communities (>50% in all cases) except when grown with BT. The absolute abundance of C. difficile remained high after three 24 h growth cycles, except for the community containing PV (Sulaiman et al. 2024 at FIG. S9c-d). In the 7-member community, C. difficile displayed a relative abundance of ˜20-50% following 24 h of growth (Sulaiman et al. 2024 at FIG. S9e). This contrasts with the low abundance of C. difficile in the glucose media (˜1 to 5%) (Sulaiman et al. 2024 at FIG. S7a).

To determine the inter-species interaction network in the presence of multiple preferred carbohydrates, we built a gLV model using a design-test-learn (DTL) cycle (Sulaiman et al. 2024 at FIG. S9f). A DTL cycle was used to account for potentially more complex interactions in the presence of a complex resource environment, which may require additional data to constrain the model parameters. Each cycle consisted of (i) Bayesian experimental design informed by prior experimental observations to select combinations of species that minimize parameter uncertainty (design), (ii) experimental characterization of sub-communities (test), and (iii) updates to the gLV model parameters based on new experimental data (learn) (Methods and Sulaiman et al. 2024 at supplementary text section 1-8 in Supplementary Information) ⁴⁴. In the initial experiment, we constructed 82 communities consisting of all possible pairwise, leave-one-out, and full communities containing the gut bacteria and individual C. difficile strains (Sulaiman et al. 2024 at Table S8, DATASET002). Using 10-fold cross-validation, the model displayed a low to moderate prediction performance of individual species (Sulaiman et al. 2024 at FIG. S9g). To select informative experimental conditions for the second DTL cycle, Bayesian experimental design based on the inferred parameter uncertainties guided the design of 94 new combinations of medium richness communities (3-6 members) (Sulaiman et al. 2024 at Table S8, DATASET003). Using these data, the prediction performance of most individual species was improved (Pearson's R=0.90 to 0.91, P<10E-05) (Sulaiman et al. 2024 at FIG. S9g). The parameter uncertainty distributions are shown in Sulaiman et al. 2024 at FIG. S10. In comparison to the media with glucose, the constrained non-zero parameters are lower in the mixed carbohydrates media (60.9%, 71.8%, 68.8%, and 67.2% for communities containing DSM, MS001, MS008, and MS014 respectively). To determine whether species predictive performance could be improved with additional data, we performed a sensitivity analysis of the model's prediction performance by varying how the training and validation data was partitioned (k in k-fold) (Sulaiman et al. 2024 at FIG. S11). The model prediction performance increased with k and saturated for most species. This implies that additional data for moderately predicted species (e.g. CH and DP) will not substantially improve the model prediction performance. Poor or moderate prediction performance could be due to insufficient variation of the particular species abundance across communities or limited flexibility of the gLV model to capture complex interaction modalities ³⁹.

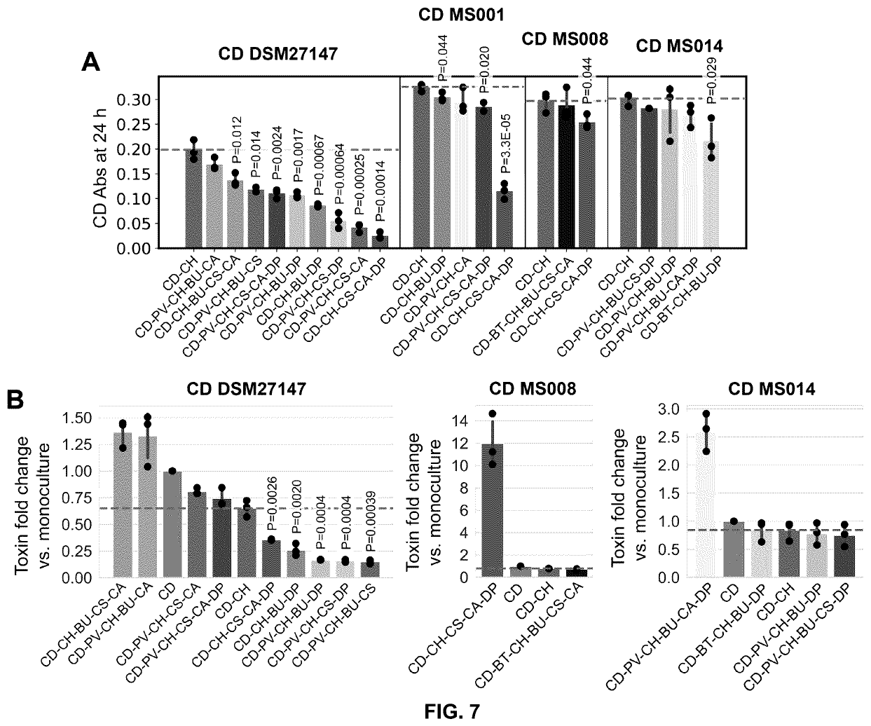

The inferred interaction networks in the mixed carbohydrates media display a higher frequency of positive interactions (4-18%) compared to media containing only glucose (2-5%) (FIG. 2E), and C. difficile displayed higher absolute abundance across communities (Sulaiman et al. 2024 at FIG. S12). While DSM 27147 exhibited the most different interaction profile in glucose media, this strain displayed similar interaction patterns to MS008 and MS014 in the mixed carbohydrates media. By contrast, MS001 displayed the largest differences in inter-species interactions in the mixed carbohydrates media than the other C. difficile strains. Thus, the differential interaction profiles between the C. difficile strains and human gut microbiota are nutrient dependent. Of 7 diverse human gut species, only CH displayed negative interactions with each C. difficile strain. Several communities used to train the model (3-6 members) containing CH displayed a higher magnitude of C. difficile inhibition than the C. difficile-CH pairwise community (FIG. 2G, FIG. 7 (A). In particular, CS, DP, CA, and PV are enriched in these communities. This suggests that the inhibitory activity of CH can be further enhanced by the presence of specific gut bacteria.

To further investigate inter-species interactions in the mixed carbohydrate media, we cultured different C. difficile strains in the sterilized spent media of the gut bacteria and fresh media as a control (Sulaiman et al. 2024 at FIG. S14a-b). Overall, the qualitative effects of the pH-adjusted conditioned media were largely consistent with the signs of the inferred gLV pairwise interaction coefficients (71% agreement compared to 32% in the non-pH-adjusted conditioned media) (Sulaiman et al. 2024 at FIG. S14c). Without pH adjustment, C. difficile growth was substantially reduced in Bacteroides spp. conditioned media due to the acidification of the environment (pH of 5.0-5.2), and this inhibition was eliminated in the pH-adjusted Bacteroides spp. conditions. Since pH changes over time in co-culture, the large variation in the initial pH of the spent media may not be physiologically relevant to microbial community interactions. Notably, C. difficile growth was reduced in CS-conditioned media but not in co-culture with CS. This inconsistency suggests that the feedback of metabolite exchange and/or metabolic niche partitioning plays a role in the C. difficile-CS pair in the mixed carbohydrates media. Although CS can utilize many of the same carbohydrates as C. difficile, CS has a wider range of carbohydrate utilization capabilities than C. difficile in the tested media (FIG. 2B). This implies that C. difficile and CS may prefer utilizing similar resources in monoculture and display distinct metabolic niches in co-culture.

Model Accurately Predicts C. difficile Inhibition Potential in Human Gut Communities

Using the model trained on all data, we forecasted the abundance of C. difficile at 24 h in all possible communities (FIGS. 2F-2G). A previous study showed a strong negative dependence between C. difficile growth and species richness in a rich media 31, consistent with a negative relationship between these variables in glucose media. However, this trend was not present in the presence of mixed carbohydrates. This suggests that high-richness communities may not universally inhibit C. difficile in environments with C. difficile preferred substrates, and the identity of the species in the community may be more impactful than the number of species.

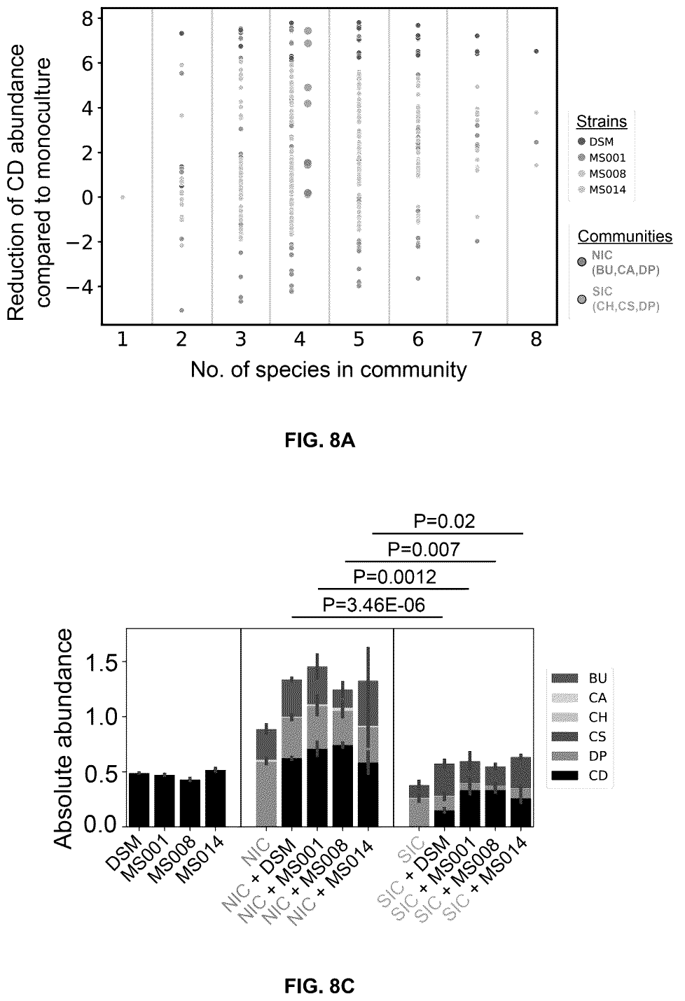

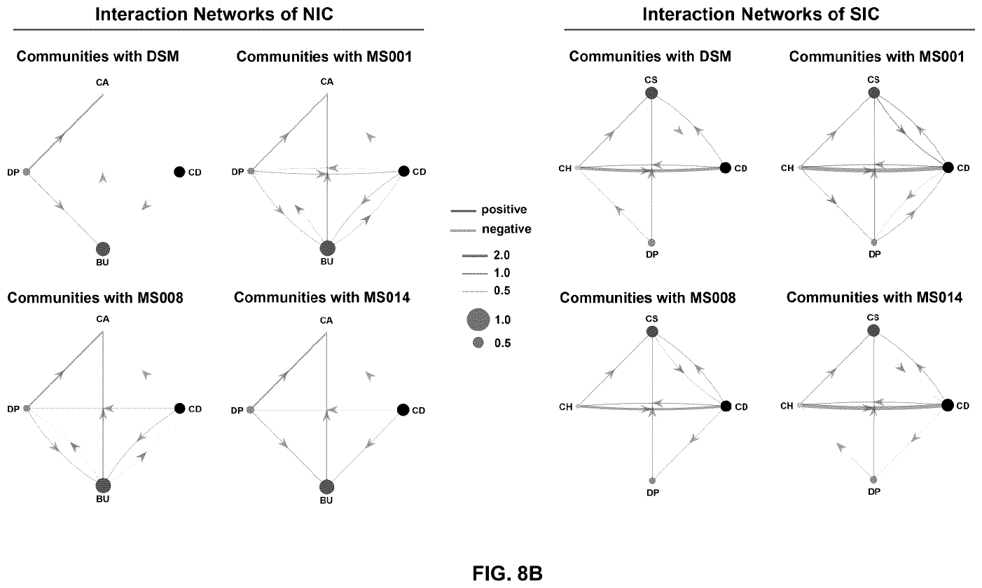

To determine if our model could design communities to inhibit C. difficile, we used our gLV model trained on community data in the mixed carbohydrates media (Sulaiman et al. 2024 at Table S8, DATASET003) to predict C. difficile abundance in all possible 2 to 8-member communities (FIG. 8A). Based on the model prediction, we selected a 3-member non-inhibitory community (NIC, consisting of BU, CA, and DP) and a strong inhibitory community (SIC, consisting of CH, CS, and DP). The NIC was selected due to its low inhibition potential of C. difficile, whereas the SIC was selected for its high inhibition potential against diverse C. difficile strains. Although CH was the only species that could strongly inhibit C. difficile in the mixed carbohydrates media, CH, CS, and DP were the three most inhibitory species in the glucose media (FIGS. 2D-2E). The interaction networks revealed sparse and almost negligible incoming negative interactions towards C. difficile in the NIC. By contrast, the SIC displayed stronger negative interactions towards C. difficile, especially from CH (FIG. 8B). To validate the model predictions, we cultured NIC and SIC in the absence and presence of different C. difficile strains (FIG. 8C). We observed that the abundance of all C. difficile strains in SIC was significantly lower than those in the NIC (˜2.1 to 4.2-fold lower), corroborating the differential inhibitory potential of the SIC and NIC communities and highlighting that the inhibition of the SIC is robust to strain-level variability. This indicates that the model could predict the C. difficile inhibition potential of different communities.

C. difficile Strains have a Differential Ability to Compete with C. scindens Over Proline

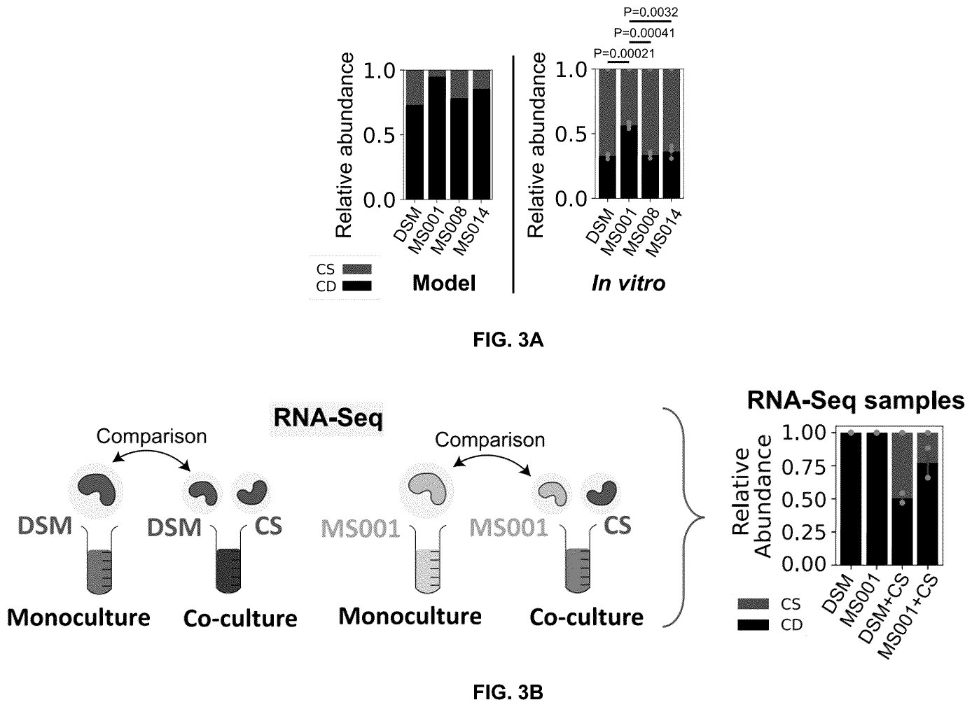

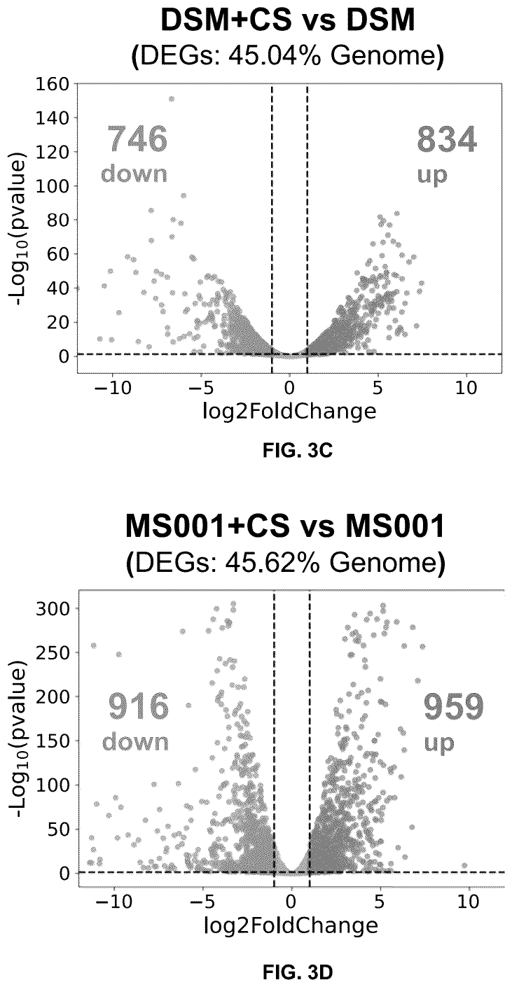

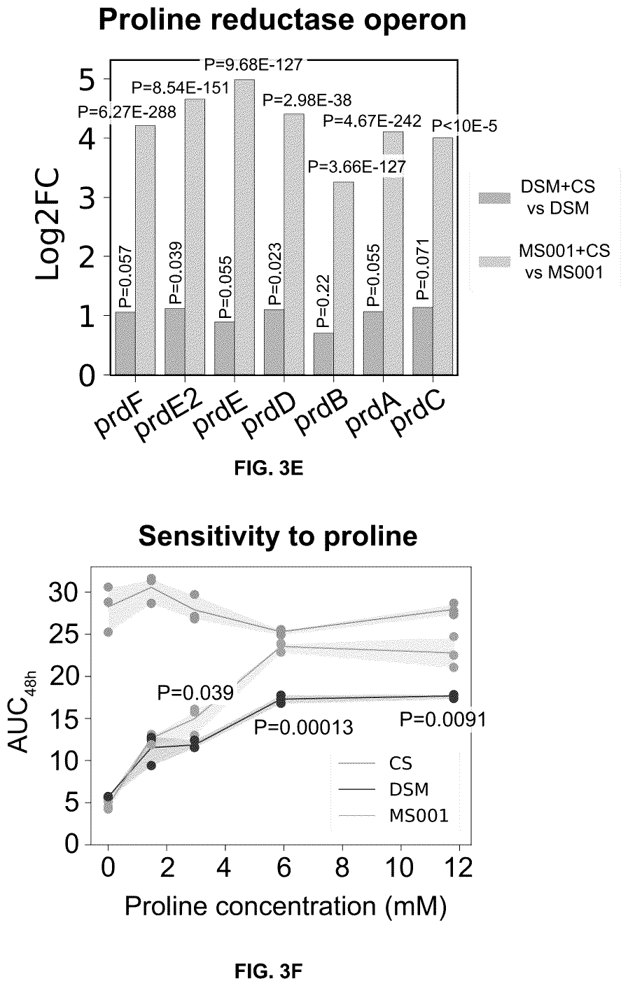

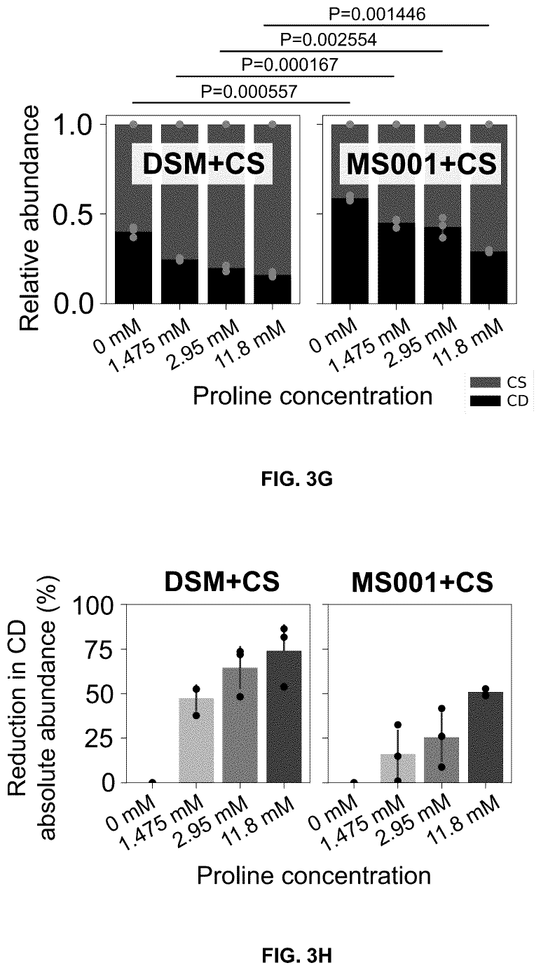

Although CS can inhibit the growth of C. difficile via competition for limiting pools of amino acids via Stickland metabolism 33, inhibition of most C. difficile strains by CS was not observed in the mixed carbohydrates media (FIG. 2E). This suggests that these C. difficile strains occupied alternative metabolic niches in co-culture with CS. The inferred interaction from CS to MS001 was larger in magnitude than to MS008 or MS014. By contrast, CS moderately inhibited the growth of the DSM strain. Model predictions of co-cultures of CS and individual C. difficile strains displayed consistent trends with independent in vitro experiments that did not inform the gLV model (FIG. 3A).

To provide insights into the transcriptional activities that mediate the observed differences in inter-species interactions, we performed genome-wide transcriptional profiling of C. difficile strains DSM27147 and MS001 in the presence and absence of CS (FIG. 3B, Sulaiman et al. 2024 at FIG. S16a). For both DSM and MS001 strains, ˜45% of transcripts were differentially expressed in the presence of CS than in monoculture, indicating that the presence of CS caused a global shift in the transcriptome of C. difficile (FIGS. 3C-3D, Sulaiman et al. 2024 at Supplementary Data Files 1-2).

To identify significant changes in transcriptional activities, we performed gene set enrichment analysis (GSEA) using Kyoto Encyclopedia of Genes and Genomes (KEGG) modules. Many biological pathways such as the amino-acid transport system, pimeloyl-ACP biosynthesis, and iron complex transport system displayed similar patterns in DSM and MS001 (Sulaiman et al. 2024 at FIG. S16b-e). In addition, both C. difficile strains up-regulated genes for mannitol utilization, consistent with the inability of CS to utilize mannitol (FIG. 2B). This implies that C. difficile and CS display niche partitioning in co-culture, thus reducing competition for limiting substrates. In addition, both strains down-regulated the grd operon which is involved in glycine utilization via Stickland metabolism. Notably, only the MS001 strain up-regulated the proline reductase (prd) genes for Stickland metabolism via the proline pathway (˜10 to 32-fold) (FIG. 3E). This implies that these C. difficile strains display differential utilization of proline in the presence of CS.

The growth of C. difficile increased with supplemented proline (FIG. 3F, Sulaiman et al. 2024 at FIG. S17a). The MS001 strain displayed a significantly larger increase in growth than the DSM strain in the presence of intermediate proline concentrations. Although there are some variations in the sequence of the prd operon genes among C. difficile isolates, their protein-coding sequences are largely similar (Sulaiman et al. 2024 at FIG. S17b-c). By contrast, variation in supplemented proline did not alter the growth of CS. This demonstrates that proline metabolism via the Stickland pathway is crucial for C. difficile growth, but not a major resource utilized by CS in monoculture. However, we observed an opposite trend in co-cultures where increasing proline concentrations reduced C. difficile growth in the community (FIG. 3G, Sulaiman et al. 2024 at FIG. S17d). These results suggest that CS competed more efficiently with C. difficile over proline in co-culture, which was distinct from its metabolic niche in monoculture. The absolute abundance of CS increased with supplemented proline only in co-culture with the MS001 strain, but not the DSM strain (Sulaiman et al. 2024 at FIG. S17d). Consistent with the monoculture data, the MS001 strain displayed higher growth than DSM in co-culture with CS (Sulaiman et al. 2024 at FIG. S17d), and its abundance was reduced to a lower degree as a function of proline compared to the DSM strain (FIG. 3H). These data suggest that MS001 can compete better with CS over limited proline to perform Stickland metabolism than DSM, consistent with the higher fold change in the expression of the prd operon (FIG. 3E). These trends are consistent with the stronger inhibition of CS by MS001 compared to DSM in the inferred gLV interaction network (FIG. 2E).

C. difficile Toxin Production in Communities is not Explained by Growth-Mediated Inter-Species Interactions

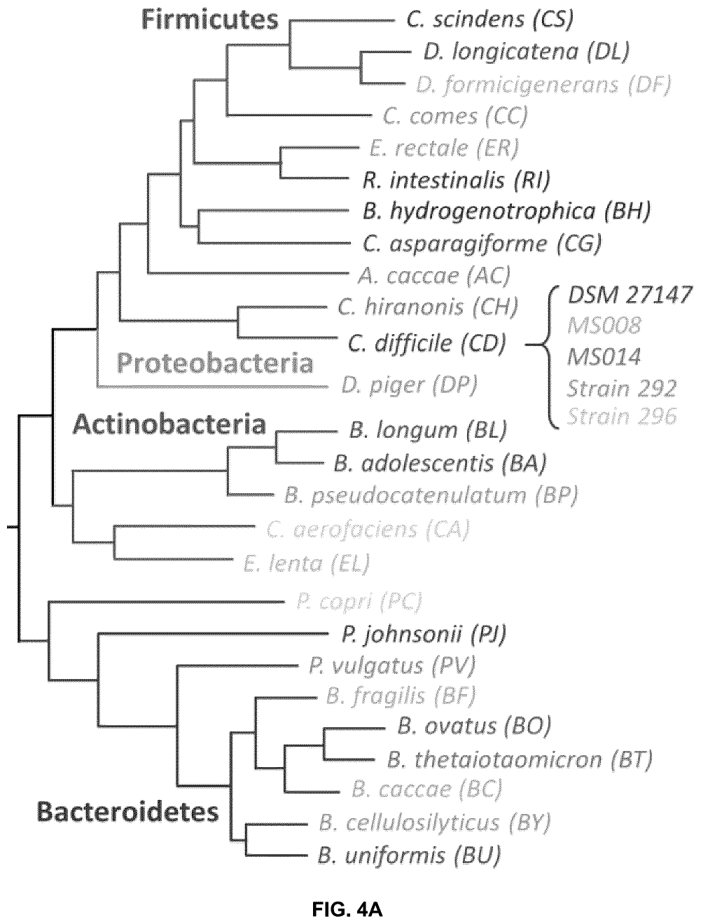

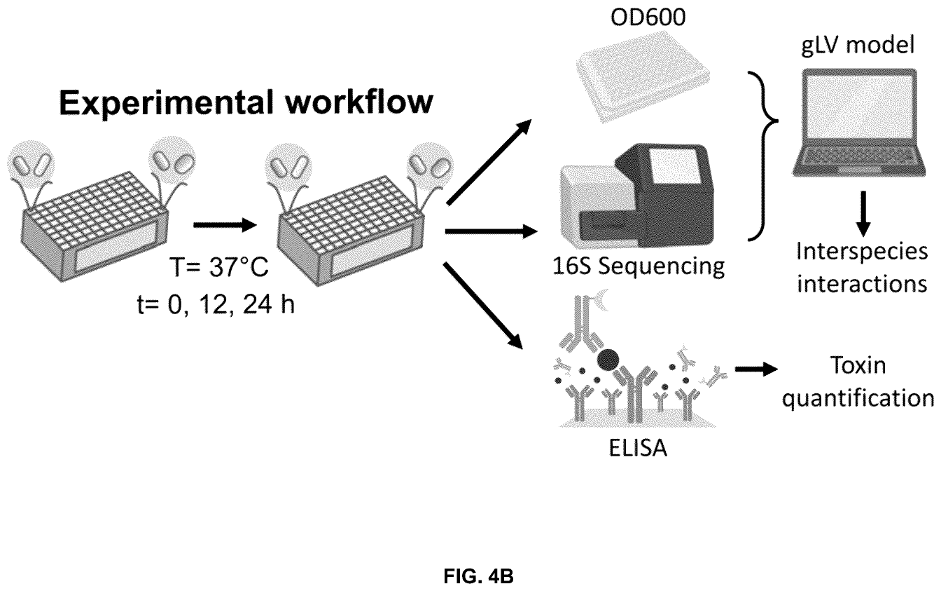

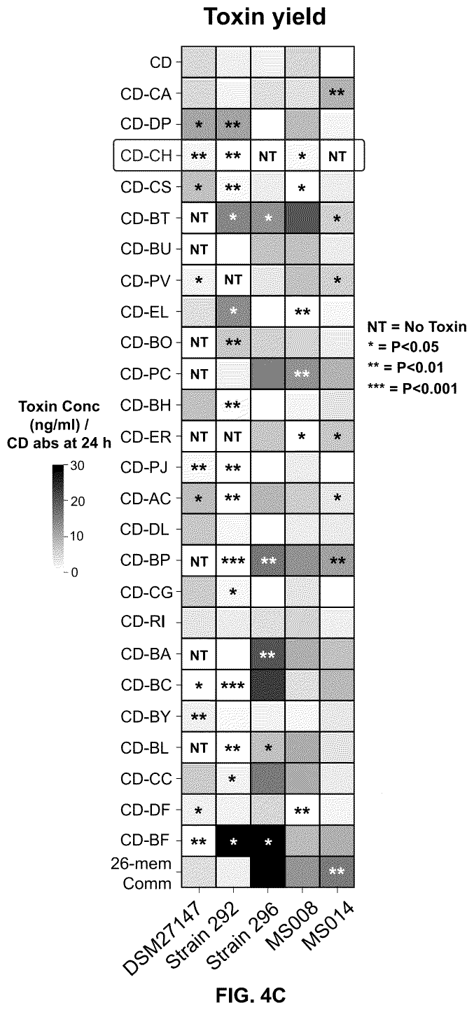

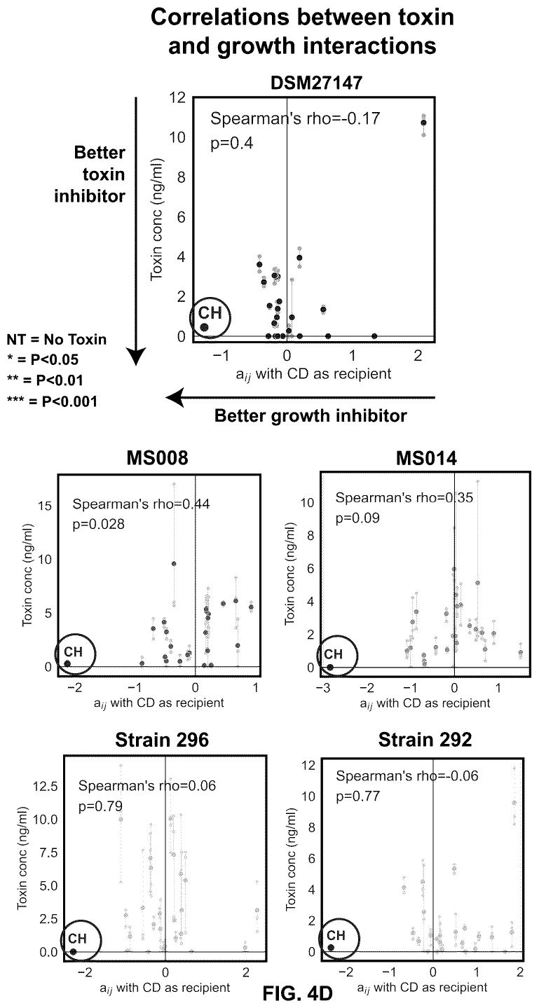

A myriad of environmental factors including specific nutrients 62-66 pH 67, and environmental stressors including alteration of the redox potential, antibiotic exposure, and temperature increase 68 shape the production of toxins in C. difficile. By modifying the environment, certain bacterial species may impact the toxin production of C. difficile 69,70. However, we lack an understanding of how toxin production is shaped by diverse human gut species. To investigate this question, we characterized C. difficile toxin expression in the presence of 25 individual diverse human gut species. Many of these species are prevalent and abundant in the human gut microbiome and are linked to human health and disease ⁴⁴ (FIG. 4A, Sulaiman et al. 2024 at FIG. S18a-b). Individual species were co-cultured with distinct C. difficile strains that we previously used to study community-level interactions (DSM27147, MS008, and MS014), as well as two other C. difficile strains isolated from healthy individuals (Strain 292 and Strain 296) which are clustered differently from the previous strains in terms of genotype and phenotype (FIGS. 1B-1D). We measured OD₆₀₀ and performed 16S sequencing to determine species absolute abundances, and end-point toxin quantification using ELISA (FIG. 4B). A gLV model was fit to the time-resolved absolute abundance data (0, 12, 24 h) to infer inter-species interactions (Sulaiman et al. 2024 at FIG. S18c-d, Sulaiman et al. 2024 at DATASET004 in Table S8). The inferred interaction parameters using this dataset displayed an informative relationship with the parameters inferred in FIG. 2E (Sulaiman et al. 2024 at DATASET003) (Sulaiman et al. 2024 at FIG. S18e).

Toxin yield (toxin concentration normalized by the C. difficile absolute abundance at 24 h) provides insight into context-dependent changes in toxin production, whereas the toxin concentration may be more physiologically relevant. In 16.2% of conditions, toxin yields were enhanced in communities than in monoculture (36.2% for toxin concentration) (FIG. 4C, Sulaiman et al. 2024 at FIG. S19a). Meanwhile, in 26.2% of conditions, toxin yields were reduced in communities compared to monoculture (25.4% for toxin concentration). Genotype and toxin production did not display an informative relationship since the similar hypervirulent strains DSM27147 and MS014 displayed very different toxin production profiles in communities. Overall, C. difficile strains exhibited substantial variability in toxin production with Strain 296, MS008, and MS014 displaying greater similarity to each other than the other strains (Spearman's rho=0.53-0.75, P=5.4E-03 to 1.1E-05) (FIG. 4C, Sulaiman et al. 2024 at S19b). These strains displayed higher toxin production in many pairwise communities (e.g. BT, BU, PV, PC, BP, BA, BC, BL, CC, and BF) and the 26-member community. The similarities in toxin production profiles were not explained by toxin protein-coding sequences (Sulaiman et al. 2024 at FIG. S19c). While Strain 296 and MS014 clustered together based on their metabolic genes, MS008 has distinct metabolic genes (Sulaiman et al. 2024 at FIG. S3a). These imply that toxin production in communities is likely impacted by regulatory networks and other cellular processes ⁷¹⁻⁷³ that are shaped by gut microbiota inter-species interactions.

Some stresses, including nutrient limitations, have been reported to induce C. difficile toxin production ^{72,74} Strong negative inter-species interactions may activate stress response networks, leading to an increase in toxin production. However, our results revealed that toxin production and the inferred pairwise gLV interaction coefficients impacting C. difficile growth in communities did not display an informative relationship (FIG. 4D), suggesting that C. difficile growth in communities was not correlated with toxin production (Sulaiman et al. 2024 at FIG. S19d). In sum, C. difficile strain-level variability and human gut microbiota inter-species interactions beyond growth were major variables shaping toxin production.

C. difficile Metabolism, Growth, and Toxin Production are Substantially Impacted by C. hiranonis

Based on the inferred inter-species interaction network, CH inhibited distinct C. difficile strains regardless of whether the nutrient environment favored C. difficile growth or not (FIGS. 2D-2E). Of 25 diverse gut bacteria, CH is the only species that robustly inhibited both C. difficile growth and toxin production of diverse C. difficile strains (FIGS. 4C-4D), highlighting its potential as a “universal” C. difficile inhibitor. This robustness of inhibitory interaction across the two nutrient environments and strain background may be attributed to the substantial metabolic niche overlap for carbohydrate utilization (FIG. 2B) and capability for amino acid Stickland metabolism. In addition, introducing C. hiranonis into communities with specific human gut species enhanced C. difficile growth and toxin inhibition than in co-culture with only C. hiranonis (FIG. 7 (A-B)).

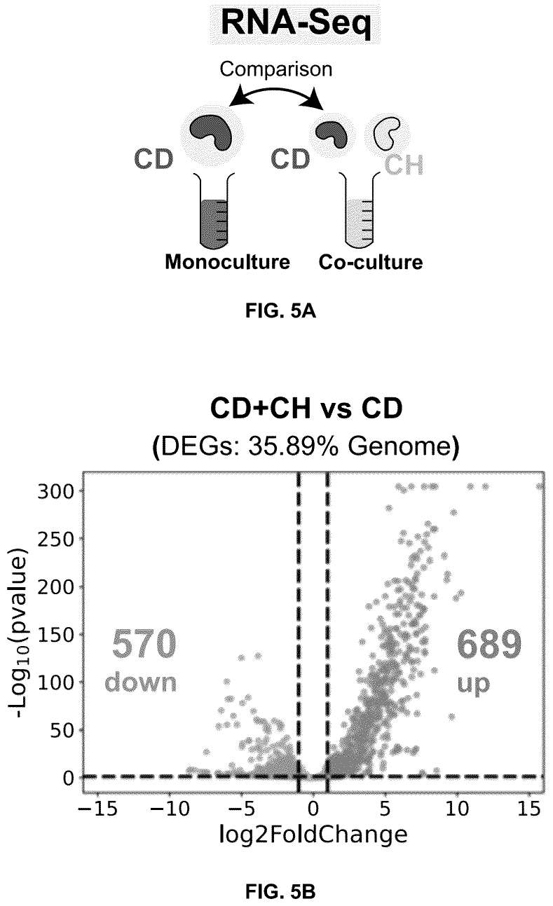

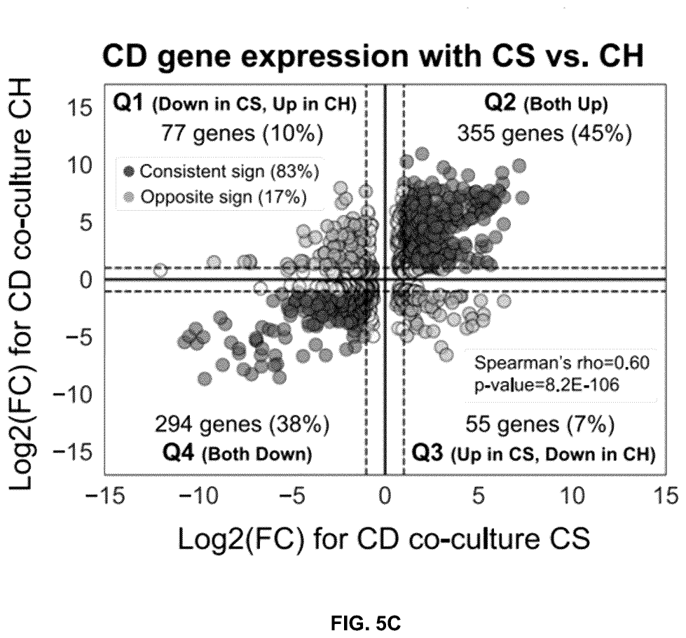

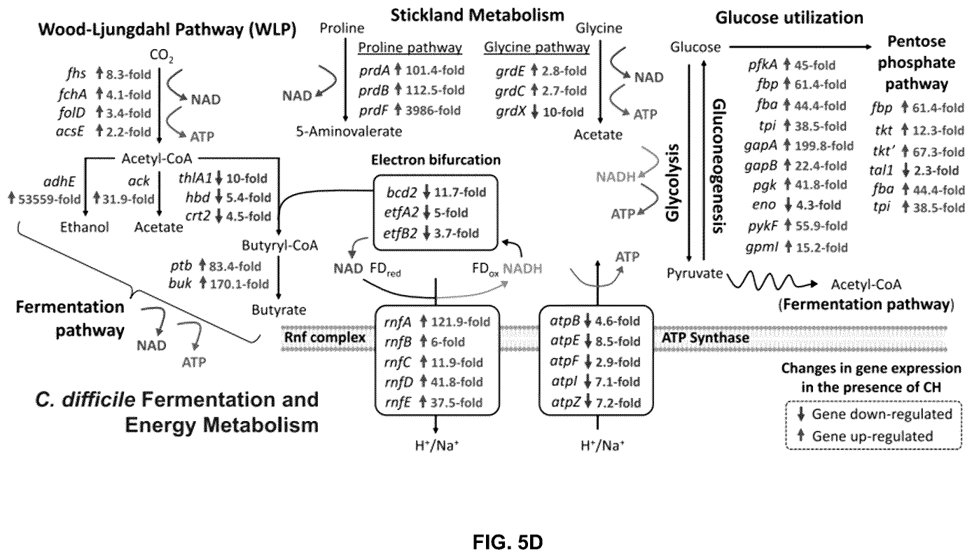

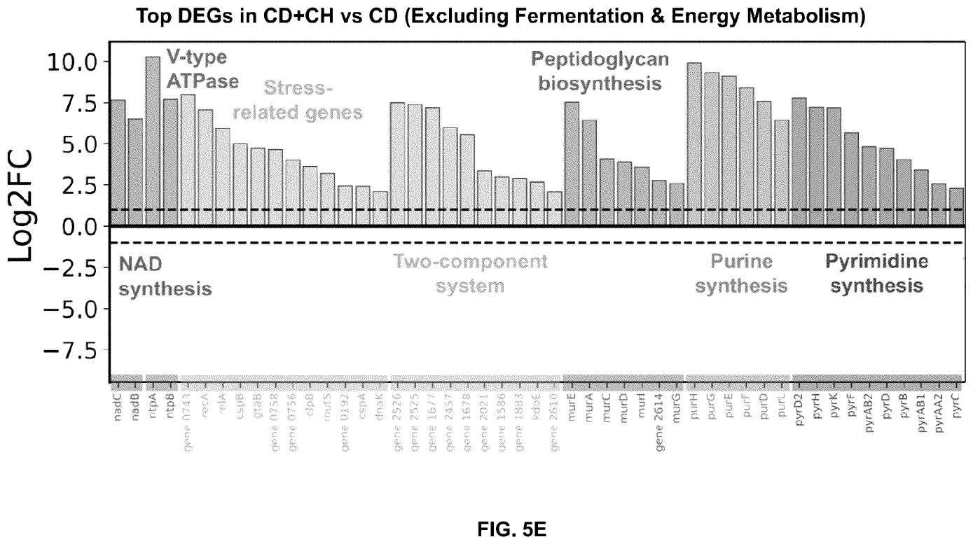

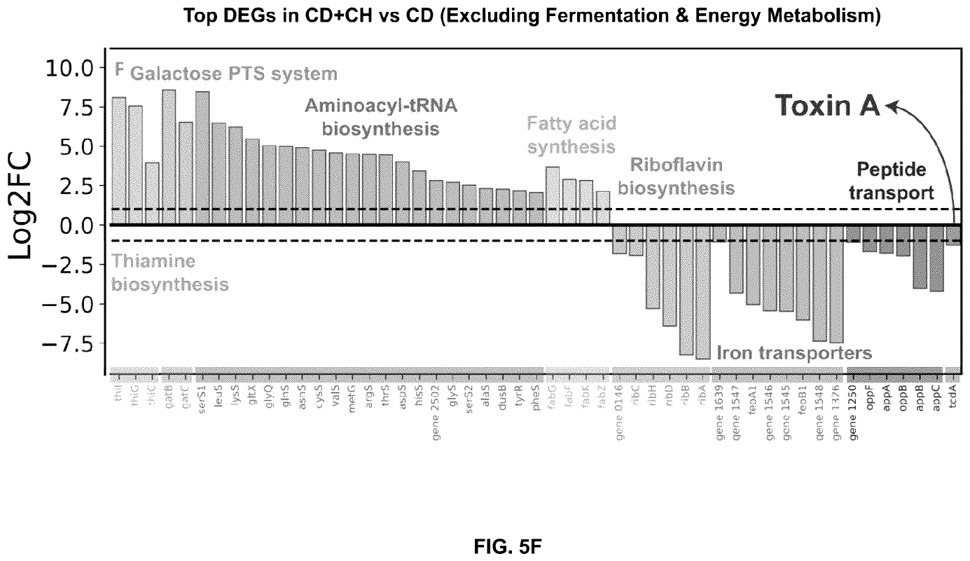

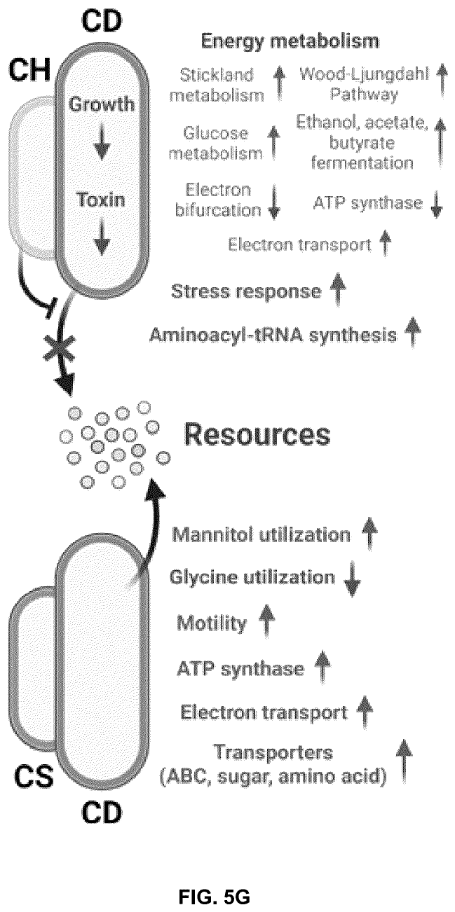

To provide insights into the mechanisms by which CH inhibits C. difficile, we performed genome-wide transcriptional profiling of C. difficile DSM27147 in the presence and absence of CH (FIG. 5A, Sulaiman et al. 2024 at FIG. S16a and FIG. S16f). In the presence of CH, 36% of C. difficile genes were differentially expressed compared to monoculture (FIG. 5B, Sulaiman et al. 2024 at Supplementary Data File 3). The transcriptional profile of C. difficile in the presence of CH was largely different compared to the co-culture with CS (17% of genes have an opposite sign of fold change) (FIG. 5C).

Notably, co-culturing with CH yielded a massive alteration in the expression of fermentation and energy metabolism genes in C. difficile (FIG. 5D). Many genes involved in glycolysis, pentose phosphate pathway, Stickland metabolism, Wood-Ljungdahl Pathway (WLP), and fermentation pathway were highly up-regulated in the presence of CH. Since ATP synthases were down-regulated, it is possible that the cells were forced to generate ATP through the aforementioned pathways to perform essential cellular functions. C. difficile couples certain fermentation pathways, such as the butyrate fermentation, to the generation of a sodium/proton gradient using electron bifurcation in combination with the membrane-spanning Rnf complex ⁷⁵. Electron bifurcation couples the NADH-dependent reduction of a substrate to the reduction of ferredoxin. The free energy resulting from the redox potential difference between ferredoxin and NAD⁺ is used to transport ions across the membrane through the Rnf complex, generating NADH in the process. Since electron bifurcating enzymes were down-regulated, Rnf complex genes were up-regulated, and glycolysis genes were highly up-regulated, C. difficile likely needed to generate NAD⁺ in the presence of CH. This could be achieved by the reductive Stickland metabolism or the WLP coupled to fermentation pathways. C. difficile heavily relies on Stickland reactions for reductive pathways ⁷⁶. When there are abundant preferred electron acceptor substrates such as proline and glycine, the WLP is not used by C. difficile. However, C. difficile uses WLP as its terminal electron sink to support growth on glucose when C. difficile lacks Stickland amino acid acceptors ⁷⁶. Therefore, the concomitant up-regulation of the proline and glycine reductases and genes involved in the WLP suggests that C. difficile competed with CH over proline and glycine and thus resorted to the WLP as an alternative electron-accepting pathway.

In addition to altering C. difficile's metabolism, CH impacted the expression of genes involved in various important cellular pathways such as stress responses (FIGS. 5E-5F, Sulaiman et al. 2024 at FIG. S16g). For instance, genes related to two-component systems that enable bacteria to adapt to diverse environmental changes, and many stress response genes including recA and relA were highly up-regulated. Consistent with the inhibition of C. difficile's toxin production in the presence of CH as measured by ELISA, the toxin A (tcdA) gene was down regulated in the presence of CH. Since C. difficile toxin expression is tightly linked with metabolic activity ⁷², toxin inhibition by CH could be associated with the massive changes in C. difficile's metabolism. In sum, CH blocked access of C. difficile to alternative resource niches and led to a global alteration in the metabolic activities of C. difficile, providing insights into mechanisms that could mediate inhibitory inter-species interactions that are robust to strain and nutrient variability (FIG. 5G). In contrast, another closely related species, CS, loses its inhibitory activity in the presence of multiple carbohydrates since C. difficile can utilize mannitol, which is not utilized by CS.

C. hiranonis Ameliorates Disease Severity in the Murine Gut in Response to Different C. difficile Strains or Nutrient Perturbations

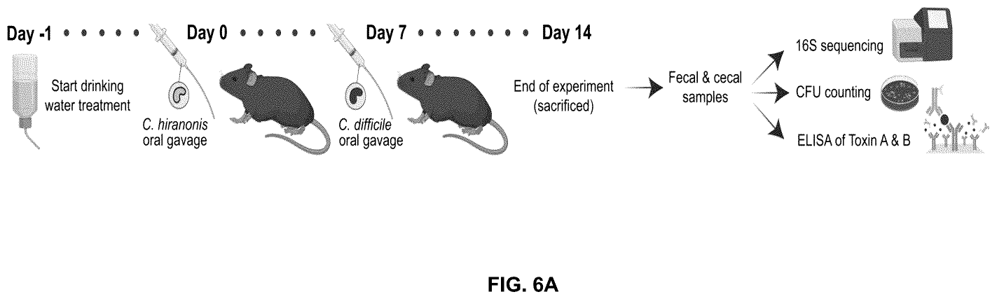

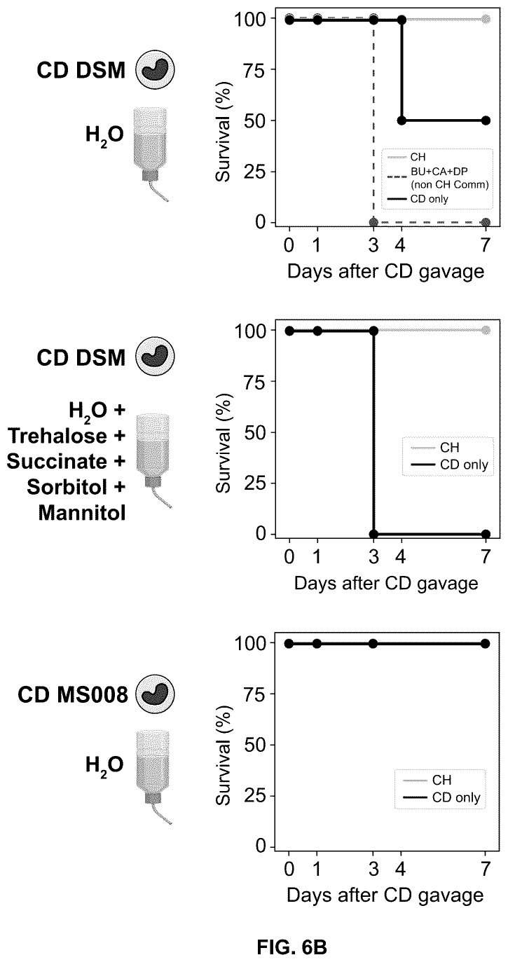

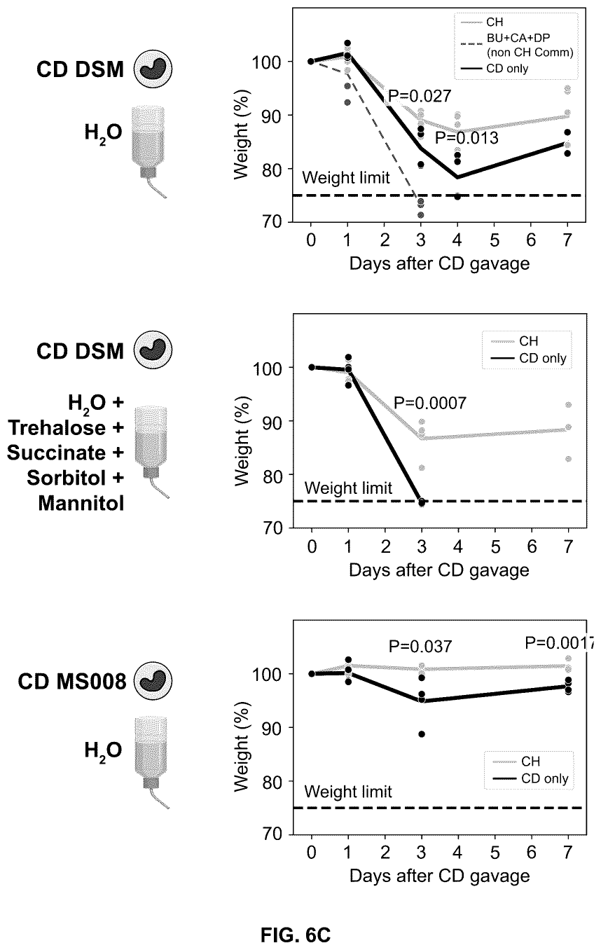

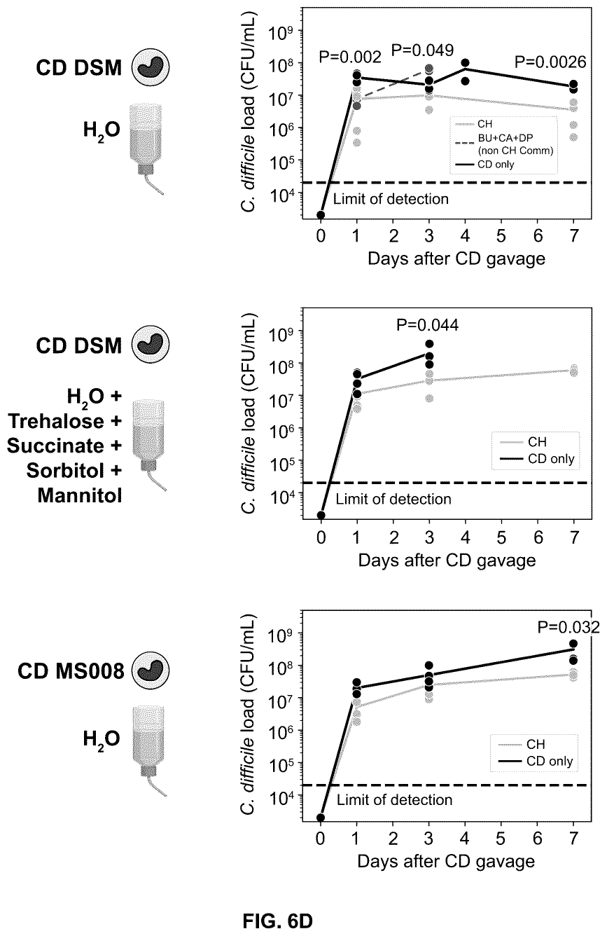

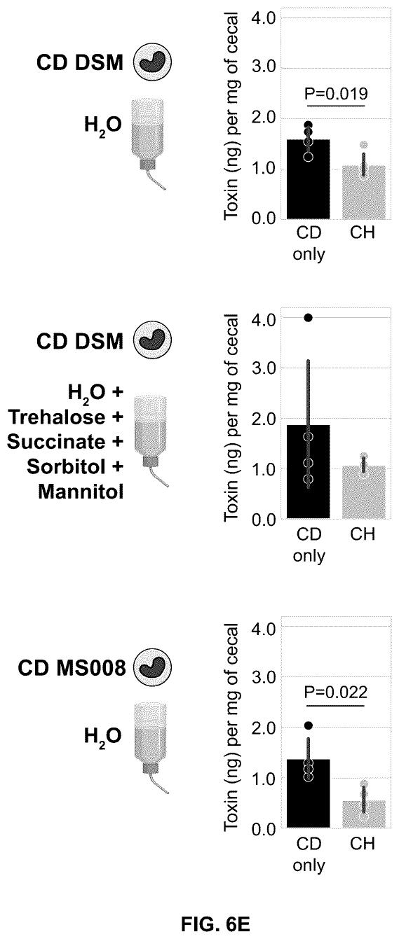

We evaluated the ability of C. hiranonis to inhibit different C. difficile strains in vivo in response to different nutrient environments. To this end, we introduced C. hiranonis into germ-free mice fed a standard assorted fiber diet (FIG. 6A). One group of mice received drinking water supplemented with C. difficile-preferred carbohydrates (trehalose, succinate, mannitol, sorbitol). The remaining nutrients in our media including galactose, GlcNAc, GalNAc, and sialic acid can be derived from host mucins ³². After one week, we orally gavaged mice with the genetically distinct hypervirulent C. difficile DSM27147 or MS008 strain (FIG. 1C-1D). These strains also display distinct toxin profiles in human gut communities (FIG. 4). As a control, we orally gavaged germ-free mice with C. difficile.

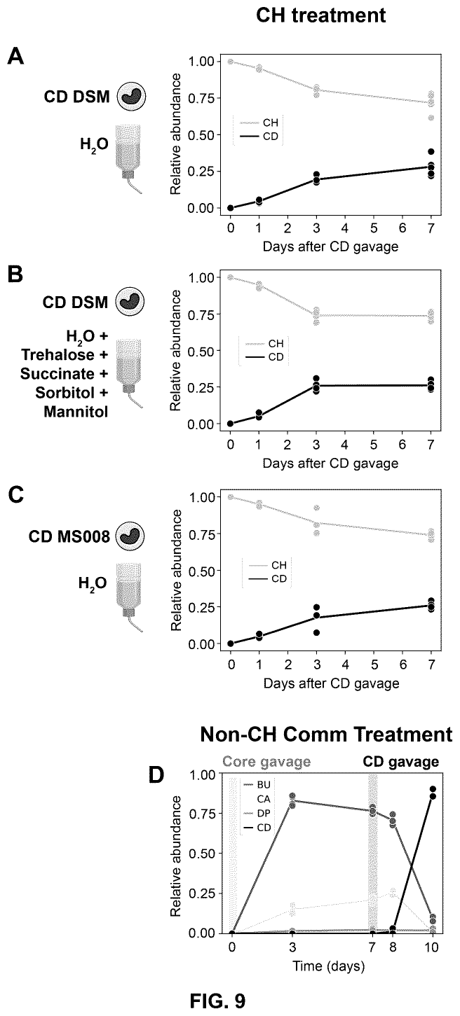

In the unmodified drinking water group, 50% of mice harboring only C. difficile died four days after gavage, whereas all mice orally gavaged with CH survived until day 7 (FIG. 6B, top). The relative reduction in weight for mice harboring CH was significantly lower than the C. difficile-only group, although both groups displayed a decreasing trend in weight over the first few days (FIG. 6C, top). To test whether the protective ability of CH is unique or due to competition with other human gut bacteria, we orally gavaged the mice with a non-inhibitory 3-member community (BU, CA, DP). This community did not inhibit the growth of C. difficile based on our in vitro data and model (FIG. 8A-8C). Mice harboring the BU-CA-DP community displayed a larger reduction in weight than the C. difficile-only group and none survived after 3 days following the introduction of C. difficile (FIGS. 6B-6C, top). Although all species in this community colonized the mice after one week of gavage, C. difficile exhibited high abundance (˜88%) after 3 days following the introduction of C. difficile (FIG. 9 (D)). This implies that the inhibitory effect of CH is not simply due to colonization of human gut bacteria and is a unique property of CH.

In the supplemented drinking water group, no mice survived after 3 days following the introduction of C. difficile in the C. difficile-only group, indicating that supplementation of C. difficile-preferred resources enhanced disease severity (FIG. 6B, middle). Consistent with the unmodified drinking water group, CH reduced the disease severity in the supplemented drinking water group (FIG. 6C, middle). Despite the ability to colonize and produce similar levels of toxin to DSM (FIGS. 6D-6E, bottom), C. difficile MS008 was not lethal even in the C. difficile-only group (FIG. 6B, bottom). This suggests that phenotypic differences beyond toxin production play a crucial role in modulating disease severity in mice. The weight of mice in the C. difficile-only group decreased by ˜5% after 3 days following the introduction of C. difficile MS008, whereas the mice that harbored CH maintained their initial weight for the duration of the experiment (FIG. 6C, bottom). Across all groups with different C. difficile strains and nutrients, the mice harboring CH displayed significantly lower C. difficile abundance than in the absence of CH (FIG. 6D), and CH displayed a high relative abundance (FIG. 9, (A-C)). In addition, toxin concentration was significantly reduced for both C. difficile strains in the presence of CH in the unmodified drinking water groups (FIG. 6E). Consistent with our in vitro results, CH robustly ameliorated the disease severity of a C. difficile challenge in a murine model in response to two different C. difficile strains and variations in nutrient environments.

### Discussion

Defined communities that have been optimized to inhibit C. difficile hold tremendous promise to overcome the limitations of FMT for treating CDI. For instance, oral consortia from VE303 (Vedanta Biosciences) has passed the phase 2 clinical trial for rCDI 11 and is currently undergoing phase 3. Robustness of anti-C. difficile activity to environmental variability is not typically considered in the design process. This potential lack of robustness may contribute to the failure of the community to successfully treat a fraction of patients (˜14% after a few months) ^{77,78}. The C. difficile inhibitory activity of defined communities may be more variable than fecal communities used during FMT due to their reduced functional redundancy, richness, and diversity ^{7,8}. Therefore, there is a need to understand how anti-C. difficile activity of human gut communities varies in response to diverse C. difficile strain backgrounds and environmental contexts (e.g. variations in diet). We used a data-driven approach to dissect interspecies interactions and toxin production of genotypically diverse C. difficile strains in human gut communities under different nutrient environments. We combined high-throughput in vitro experiments with computational modeling to deduce interaction networks impacting each C. difficile strain in different media conditions. We showed that C. difficile strain variation could directly or indirectly shape interspecies interactions of human gut microbiota. In addition, strain-level variability has a major impact on toxin production in communities, adding another layer of complexity to the design of robust anti-C. difficile consortia. The nutrient environment also plays a key role in shaping the interactions between C. difficile and the gut communities. Although it has been reported that C. difficile inhibition is prevalent in a rich and undefined media ³¹, we showed that it is sparse when there are multiple preferred carbohydrates for C. difficile. Our study showcases our quantitative systems-biology approach to map context-dependent interactions and provides insights into the mechanisms that could enhance the robustness of inhibition across strains and environments. Based on our results, interactions that lead to global shifts in metabolism and other cellular processes may exhibit greater robustness to environmental variability. More broadly, this framework that considers robustness as a feature could be applied to the design of anti-pathogen bacterial therapeutics.

Certain bacteria in the gut have been reported to increase C. difficile toxin production and enhance their fitness and virulence in vivo, such as the opportunistic pathogen Enterococcus faecalis ⁷⁰. Some metabolites produced by gut microbes such as butyrate could also increase C. difficile toxin, albeit moderately ⁷⁹. However, we found that the enhancement of C. difficile toxin is sparse among human gut commensals (toxin production per unit biomass is enhanced in only ˜16% of all communities compared to monocultures). In addition, strain-level variability played a larger role in toxin production in communities than inferred gLV growth-mediated inter-species interactions. Since toxin production is tightly linked with metabolism ^{73,80}, genotypic variations among C. difficile strains would impact their toxin production profiles. The lack of an informative relationship between growth-mediated inter-species interactions and toxin production suggests that inhibiting C. difficile growth may not always protect against CDI unless C. difficile is excluded from the community. Thus, the identification of C. difficile inhibitors should consider both inhibition of growth and toxin production. Further, we discovered that C. difficile strains with similar hypervirulent genotypes (DSM 27147 and MS014) have different toxin production profiles in communities. By contrast, an isolate from a healthy individual (Strain 296) has a similar toxin production profile with genetically distinct isolates from patients with CDI (MS008 and MS014) (FIG. 4C, Sulaiman et al. 2024 at FIG. S19a-b). This indicates that rather than the genotype of C. difficile alone, community context is a major variable shaping C. difficile toxin production.

Of the 7 gut bacteria used to study community interactions, CS and CH are the only two species that can utilize amino acids to perform Stickland metabolism, similar to C. difficile. In the media supplemented with only glucose as a sole carbohydrate, CS and CH have a stronger magnitude of C. difficile inhibition compared to the other species (FIG. 2D). These inhibitory interactions may stem from competition over Stickland amino acids in addition to glucose, whereas the other gut bacteria only compete for glucose. Previous work has shown that introducing Stickland amino acid competitors can protect mice from CDI ³³. In sum, competition over Stickland amino acids is an attractive strategy to enhance inhibition against C. difficile. However, in the media containing multiple carbohydrates, CH is the only species that can inhibit C. difficile whereas CS lost this inhibition capability (FIG. 2E). In a different rich media, CH inhibition of C. difficile was proposed to arise partially from resource competition and not via external pH change or extracellular protein release ³¹. Our results go beyond this study by demonstrating that CH suppresses the growth and toxin production of diverse C. difficile strains in two distinct nutrient environments (FIGS. 2A-G and 4A-D), yields a massive change in the metabolic activity of C. difficile, and reduces disease severity in germ-free mice harboring different C. difficile strains and across different nutrient environments (FIGS. 5A-5G and 6A-E). Although to our knowledge there is no evidence regarding the role of CH on CDI outcomes in humans, the presence of CH is negatively associated with C. difficile colonization in dogs and cats ⁸¹⁻⁸³.

A key question is how CH maintains its inhibitory effect on C. difficile when provided with multiple C. difficile-preferred carbohydrates, whereas the inhibitory capability is abolished for CS. Since CH and CS are closely related, we would expect a similar transcriptional response in C. difficile in the presence of these two species. Genome-wide transcriptional profiling revealed that C. difficile exhibited a substantial difference in gene expression in the presence of CH and CS (FIG. 5C). These data provided insights into the unique transcriptional signature of CH's inhibition mechanism, which was not observed in the presence of CS. Although our results support the hypothesis that C. difficile competes for Stickland amino acids with CS, C. difficile could switch to mannitol as an alternative nutrient source, which cannot be utilized by CS (FIG. 5G). By contrast, CH and C. difficile share highly similar metabolic niches, which may substantially limit the available resources for C. difficile. Therefore, C. difficile increased expression of enzymes in core energy-generating metabolic pathways in the presence of CH, including glycolysis, pentose phosphate pathway, Stickland metabolism, Wood-Ljungdahl Pathway (WLP), and fermentation (acetate, ethanol, and butyrate production) (FIG. 5D), which were not observed when CS was present. Because C. difficile normally favors Stickland fermentation over WLP as their main electron-accepting pathway, the activation of WLP suggests that CH successfully competes for reductive Stickland amino acids and forces C. difficile to use WLP as their alternative electron sink ⁷⁶. These massive alterations in C. difficile core metabolism also impact virulence such as toxin production. Further, C. difficile upregulated stressed-related pathways (FIGS. 5E-5F), which were not observed in the presence of CS (Sulaiman et al. 2024 at FIG. S16d-e). Beyond resource competition, CH may produce an antimicrobial targeting C. difficile as previously hypothesized ³¹ that contributes to this unique transcriptional response. Future work could mine the biosynthetic gene clusters in CH for potential antimicrobial compounds and perform targeted and untargeted metabolomics to provide deeper insights into the mechanisms of inter-species interaction.

We extensively characterized the robustness of CH's anti-C. difficile activity in vitro to variation in community context, nutrient environment, and strains and in vivo to two genetically distinct strains and nutrient perturbations. However, we did not characterize the effects of other human gut communities containing CH on disease severity in germ-free mice. Our in vitro results indicate that specific human gut communities displayed larger C. difficile growth inhibition and lower toxin production across multiple C. difficile strains (FIG. 7, FIGS. 8A-8C). Future work will characterize these communities in the mammalian gut to confirm that community-level interactions can further reduce C. difficile disease severity or enable clearance. For example, while we did not identify growth-promoting interactions for CH (FIGS. 2D and 2E), interactions are predicted to enhance the inhibition of toxin production by CH (FIG. 7 (B)). In addition, we introduced vegetative cells into the murine gut and in the in vitro experiments as opposed to spores for consistency. Spores are the primary infectious form involved in the transmission and initiation of infection ^{84,85}. However, spores need to germinate into vegetative cells to colonize the human gut and produce toxins to cause infections. Future work could develop predictive computational models that capture the dynamics of sporulation and germination. This could provide deeper insights into how community context shapes the sporulation and germination rates of C. difficile.

A grand challenge for microbiome engineering is the rational design of microbial communities as living therapeutics for treating multiple human diseases involving alterations in the human gut microbiome. For CDI, a potential driver of the efficacy of FMT is the high richness and diversity of species in the fecal samples, which could repopulate the gut flora and restore colonization resistance. This is further supported by the fact that most of the products with successful outcomes in clinical trials so far are communities derived from stool samples, thus having high species richness ⁸⁶. However, due to heavy reliance on donors, these stool-derived communities suffer from batch-to-batch variations and are designed without any knowledge of molecular mechanisms of C. difficile inhibition. This could be overcome by using defined communities that are standardized and optimized to inhibit C. difficile. However, the number of strains in a bacterial therapeutic currently scales with manufacturing cost. Our study shows that in the media with multiple C. difficile-preferred carbohydrates that have been shown to increase in abundance in a perturbed gut ^{45,48,58-61}, species richness is no longer a strong determinant of C. difficile inhibition, but rather the identity of the species in the community (FIG. 2G). Therefore, it is conceivable that small bacterial communities with high anti-C. difficile activity that is robust to environmental variability could be identified. We identified CH as a “universal” C. difficile growth and toxin inhibitor of genotypically diverse C. difficile strains and nutrient environments. Therefore, CH may represent a unique class of species that could be used to build a robust anti-C. difficile bacterial therapeutics to environmental variability. Species that combined with CH in communities further enhance the anti-C. difficile activity and robustness to environmental variability in the mammalian gut.

### Methods

### Strain, Media, and Growth Conditions

The strains used in this work were obtained from the sources listed in Sulaiman et al. 2024 at Table S1. There are a total of 18 C. difficile isolates. Nine were obtained from diseased patients who were diagnosed and treated for C. difficile infection (CDI) at the UW-Madison Hospital ⁸⁷. These isolates were subjected to C. difficile nucleic acid amplification test (NAAT) (GeneXpert) via admission stool sample, and bacterial identification was confirmed via sequencing of the 16S rRNA gene. The other nine isolates were obtained from healthy individuals from the Winning the War on Antibiotic Resistance (WARRIOR) project ⁸⁸. Briefly, the WARRIOR project collects biological specimens, including nasal, oral, and skin swabs and saliva and stool samples, along with extensive data on diet and MDRO risk factors, as an ancillary study of the Survey of the Health of Wisconsin (SHOW) ⁸⁹. WARRIOR participants include 600 randomly selected Wisconsin residents aged 18 and over, and C. difficile isolates were identified by anaerobic inoculation of stool samples in C. difficile Brucella Broth and then plated on Brucella agar plates. Colonies with correct morphology were identified using Gram staining and catalase testing. The presence of toxin genes was assessed using an in-house PCR assay and bacterial identification was confirmed via sequencing of the 16S rRNA gene.

Single-use glycerol stocks were prepared as described previously ⁴⁴. The media used in this work included anaerobic basal broth (ABB, Oxoid) for growing starter cultures, and in-house defined media (DM) for all experiments. DM29 is a defined media without any carbohydrate source (recipe listed in Sulaiman et al. 2024 at Table S2) and was formulated to support the growth of phylogenetically diverse human gut bacteria ⁴⁴. This media has been used to study human gut microbiota inter-species in previous studies ^{90,91}. Supplemented single carbohydrates were added to a final concentration of 5 g/L in DM29. To mirror a perturbed human gut, we modified DM29 by supplementing with 2 g/L of glucose, sorbitol, mannitol, trehalose, succinate, galactose, GalNAc, GlcNAc and sialic acid (mixed carbohydrate media). Since previous studies demonstrated a diversity of metabolomic and microbiota outcomes following perturbation with antibiotics, we incorporated multiple carbohydrates that have been shown to increase in abundance in a perturbed gut based on multiple studies ^{45,48,58-61}.

For all experiments, cells were cultured in an anaerobic chamber (Coy Lab products) with an atmosphere of 2.0±0.5% H₂, 15±1% CO₂, and balance N₂ at 37° C. Starter cultures were inoculated by adding 200 μL of a single-use 25% glycerol stock to 5 mL of anaerobic basal broth media (ABB) and grown at 37° C. without shaking.

Growth Characterization in Media with Different Carbohydrate Sources

Starter cultures of C. difficile isolates and gut commensal bacteria were prepared. The cell pellets from starter cultures were collected by centrifugation at 3,000×g for 10 min, and then washed with DM29 media. The washed cell pellets were resuspended into DM29 media to a final OD₆₀₀ of approximately 0.1. These cultures were inoculated into a 96-well plate (Greiner Bio-One) containing DM29 supplemented with specific carbohydrate sources at a concentration of 5 g/L to an initial OD₆₀₀ of 0.01 (3 biological replicates for each strain). These plates were covered with a gas-permeable seal (Breathe-Easy® sealing membrane) and incubated at 37° C. anaerobically. Cell growth determined by OD₆₀₀ was monitored using Tecan Infinite Pro F200 microplate reader every 3 h using robotic manipulator arm (RoMa) integrated with our Tecan Freedom Evo 100 instrument.

### Logistic Growth Model

The logistic growth model was used to describe C. difficile population growth dynamics in monoculture experiments. The logistic growth model for species i takes the following form:

$$\frac{d {x}_{i}}{d t} = {x}_{i} ( {r}_{i} - \frac{{r}_{i}}{{K}_{i}} {x}_{i} ) \qquad ( 1 )$$

where xᵢ represents absolute abundance of species i, parameter rᵢ denotes the maximum growth rate, and Kᵢ denotes the carrying capacity. The logistic growth model does not capture non-monotonic growth responses and lag phases in growth. To accurately determine the growth rate, we identified time points where the OD₆₀₀ decreased >10% between two sequential time points. In this case, all time points beyond this substantial decrease were omitted from the analysis, thus eliminating the declining OD₆₀₀ over time from the model fitting procedure. The steady-state solution of the model is the carrying capacity (Kᵢ) (i.e. the value of xᵢ when

$$\frac{d {x}_{i}}{d t}$$

equals 0). To identify the lag phase, we used a threshold of 120% of the initial OD₆₀₀ (OD₆₀₀ at t=0) and eliminated these time points from the model fitting procedure. A custom MATLAB script was used to estimate the parameters θᵢ=[rᵢ, Kᵢ] in the logistic growth model. For each species i, the model was fit to experimental data and used L2 regularization to reduce overfitting to the data. Specifically, given a series of m experimental OD₆₀₀ measurements, xᵢ=[x_{i,1}, . . . , x_{i,m}], and a time series of OD₆₀₀ simulated using parameter θᵢ at the same time intervals, \hat{x}ᵢ(θᵢ)=[\hat{x}_{i,1}(θᵢ), . . . , \hat{x}_{i,m}(θᵢ)], the optimization scheme minimizes the cost function:

$${C ( {θ}_{i} ) = {\vert ( {θ}_{i} ) - {X}_{i} \vert}_{2} + λ \vert {θ}_{i} \vert}_{2} \qquad ( 2 )$$

where λ is the L2 regularization parameter, which was set to be 0.02 for all species, and |⋅|₂ indicates vector 2-norm. Solutions to the logistic growth model were obtained using the ode15s solver and the optimization problem was solved using FMINCON in MATLAB (R2022a).
Fluorescence Microscopy of C. difficile

Cell pellets from starter cultures of multiple strains of C. difficile were collected by centrifugation at 3,000×g for 10 min, and then washed with DM29 media. The washed cell pellets were resuspended into DM29 media to a final OD₆₀₀ of approximately 0.1. These cultures were inoculated into new culture tubes containing either DM29 media or DM29 supplemented with 5 g/L glucose to an initial OD₆₀₀ of 0.01 by adding 500 μl of washed starter cultures to 4.5 mL media. After 16 h and 40 h of growth, 100 μl aliquots were stained with SYBR Green, and imaged using a microscope (Nikon Eclipse Ti-E inverted microscope) using a 20× dry objective with appropriate filter sets. Images were captured with Photometrics CoolSNAP Dyno CCD camera using NIS-Elements version 4.51.00.

Whole Genome Sequencing of C. difficile Isolates

We performed whole genome sequencing of C. difficile DSM 27147 and 18 isolates used in this study. Strains were cultured from a single colony to OD₆₀₀ of 0.3, and then centrifuged to obtain the cell pellets. Genomic DNA was extracted using Qiagen DNeasy Blood and Tissue Kit according to the manufacturer's protocol. The harvested DNA was detected by agarose gel electrophoresis and quantified by a Qubit fluorometer. The genomic DNA was sent to SeqCenter (Pittsburgh, PA, USA) for paired-ends Illumina sequencing. Sample libraries were prepared using the Illumina DNA Prep kit and IDT 10 bp UDI indices, and sequenced on an Illumina NextSeq 2000, producing 2×151 bp reads. Demultiplexing, quality control, and adapter trimming were performed with bcl-convert (v3.9.3) Illumina software. The clean bases of each sample are ˜1 billion bp. The WGS raw data is accessible in BioProject PRJNA902807.

### Whole-Genome Sequencing Data Analysis

SPAdes Genome Assembler 92 was used to assemble contigs from the whole-genome sequencing data with the following parameters: spades.py --pe1-1 (forward read fastq file) --pe1-2 (reverse read fastq file) --isolate -o (output name). The --isolate option was used due to the high-coverage sequencing data. We then used FastANI ⁹³ to compute the whole-genome Average Nucleotide Identity (ANI) values between pairs of isolates, which is defined as the mean nucleotide identity of orthologous gene pairs shared between two microbial genomes. The following parameters were used: fastANI --ql (list of contigs. fasta files of all isolates from SPAdes) --rl (list of contigs.fasta files of all isolates from SPAdes) -matrix -o (output name). The newly sequenced genomes are high-quality drafts with a low number of contigs (median 61 [range 40-458]) and high N50 (median 285,062 [range 146,596-782, 135]) (Sulaiman et al. 2024 at Table S3).

Annotation of the contigs was performed using DFAST ⁹⁴. For further comparative genomic analyses, the gene content across 19 C. difficile strains was analyzed by clustering all predicted coding sequences into orthologous groups ⁹⁵ (Sulaiman et al. 2024 at FIG. S3b). Clustering of gene orthologs was carried out using ProteinOrtho6 ⁹⁵ across variable coverage and identity settings using BlastP for alignment. Distributions of OGs show a high degree of strain variability with many genes in a limited subset of strains (Sulaiman et al. 2024 at FIG. S3c).

To determine Gene Ontology (GO), we used BlastP of the NCBI Blast Suite ⁹⁶ of proteins from all C. difficile strains that exist in UniProt database (downloaded from UniProtKB on 10 Nov. 2022) at 1E-3 E-value cutoff. Following BlastP, GO information including the biological process, molecular function, and cellular compartment of each protein was extracted from UniProt. To align the sequence of specific genes such as Toxin A (tcdA), Toxin B (tcdB), RNA polymerase (rpoB, rpoB′), we used Clustal Omega multiple sequence alignment tool ⁹⁷.

We evaluated the genetic diversity of our C. difficile strains in the context of the other 118 publicly available C. difficile genomes (Sulaiman et al. 2024 at Table S4). Phylogenomic analysis was performed using GToTree ⁹⁸ on the C. difficile isolates in addition to 118 publicly available genomes. SPAdes FASTA files were used as inputs to GToTree analysis and the resulting tree was visualized using the Interactive Tree of Life web-based tool ⁹⁹. Our isolates span 64% of the C. difficile phylogeny of this dataset (9 of 14 major tree branches) (Sulaiman et al. 2024 at FIG. S3d).

To get the relative copy number of the genes in each isolate, the Illumina paired-end reads were aligned to the gene list from DFAST using Bowtie2 ¹⁰⁰. The detection of conjugative systems was performed using CONJScan ¹⁰¹ module of MacSyFinder. The detection of phages was performed using VirSorter ¹⁰².

### Construction of Strain-Specific Genome-Scale Metabolic Models to Evaluate Changes in Metabolism

Raw sequencing data was first preprocessed using fastp 0.22.0 ¹⁰³, trimming the first 5 base pairs at the 5′ end and trimming the 3′ end with a sliding window approach, maintaining a minimum quality score of 20. Reads shorter than 60 base pairs were omitted. In this analysis, 85%-95% of reads passed all filters across samples, yielding 2.9M to 7.1M reads per sample. Preprocessed reads were assembled using MEGAHIT 1.2.9 ¹⁰⁴ using default k-mer sizes and a minimum contig length of 1000 base pairs. Completeness and contamination were assessed using CheckM2 1.0.1 ¹⁰⁵ yielding completeness of >99.9% for all assemblies while maintaining contamination below 1.5%. The identity of bacterial species was verified using the GTDB toolkit 2.1.0 ¹⁰⁶ using the database version 207. All assemblies were classified as Clostridioides difficile by average nucleotide identity and placement in the GTDB reference tree.

De novo gene predictions of the assemblies were performed by Prodigal 2.6.3 ¹⁰⁷. Metabolic draft models were built using CarveMe 1.5.2 ¹⁰⁸ from the isolate gene predictions using DIAMOND 2.1.6 ¹⁰⁹ with additional options of “--more-sensitive -top 10”. Media composition was translated by manual mapping to the BiGG database ¹¹⁰. Salts were decomposed into their aqueous phase ions to mimic the effect of hydrolysis in the translated medium. Draft models were then gapfilled to be able to grow on the mapped media. During gapfilling, no more than 10 new reactions and 6 new metabolites were added to each model. Model quality was assessed using MEMOTE 0.13.0 ¹¹¹. Metabolic reaction content was assessed using the “metabolic_dist” function from MICOM 0.32.5 ¹¹² where metabolic distances were calculated by the Jaccard distance of metabolic reaction absence/presence (1−shared reactions/total reactions) for each pair of reconstructed models.

### Bacterial Genomic DNA Extraction for 16S Amplicon Sequencing

All the genomic DNA (gDNA) extraction and next-generation sequencing (NGS) sample preparation were performed as described previously ^{31,44}. Bacterial gDNA extractions were carried out using a modified version of the Qiagen DNeasy Blood and Tissue Kit protocol in 96-well plates. Briefly, cell pellets were resuspended in 180-μl enzymatic lysis buffer containing 20 mg/ml lysozyme (Sigma-Aldrich), 20 mM Tris-HCl pH 8 (Invitrogen), 2 mM EDTA (Sigma-Aldrich), and 1.2% Triton X-100 (Sigma-Aldrich), and then incubated at 37° C. at 600 RPM for 30 min. Samples were treated with 25 μL 20 mg/ml Proteinase K (VWR) and 200 μL buffer AL (Qiagen), mixed by pipette, and then incubated at 56° C. at 600 RPM for 30 min. Samples were treated with 200 μL 200 proof ethanol (Koptec), mixed by pipette, and transferred to 96-well nucleic acid binding plates (Pall). After washing with 500 μL buffer AW1 and AW2 (Qiagen), a vacuum was applied for 10 min to dry excess ethanol. Genomic DNA was eluted with 110 μL buffer AE (Qiagen) preheated to 56° C. and then stored at −20° C.

Genomic DNA concentrations were measured using the Quant-iT™ dsDNA Assay Kit (Invitrogen) with a 6-point DNA standard curve (0, 0.5, 1, 2, 4, 6 ng/μL biotium). We diluted 1 μL of the samples and 5 μL of the standards into 95 μL of 1×SYBR green (Invitrogen) in TE buffer and mixed by pipetting. Fluorescence was measured on a Tecan Spark microplate reader using an excitation/emission of 485/535 nm. Genomic DNA was then normalized to 2 ng/μL by diluting in molecular grade water (VWR International) using a Tecan Evo 100 Liquid Handling Robot.

Dual-indexed primers for multiplexed amplicon sequencing of the V3-V4 region of the 16S rRNA gene were designed as described previously ^{38,44}. PCR was performed on the normalized gDNA as template with Phusion High-Fidelity DNA Polymerase (Thermo Fisher) for 25 cycles with 0.05 μM of each primer. Samples were pooled by plate, purified using the DNA Clean & Concentrator™-5 kit (Zymo) and eluted in water, quantified by NanoDrop, and combined in equal proportions into a library. The library was quantified using the Qubit 1×HS Assay (Invitrogen), diluted to 4.2 nM, and loaded at 10 pM onto Illumina MiSeq platform for 300-bp paired-end sequencing using MiSeq Reagent Kit v2 (500-cycle), or loaded at 21 pM using MiSeq Reagent Kit v3 (600-cycle) depending on the desired sequencing reads.

16S rRNA Amplicon Sequencing Data Analysis to Determine Community Composition

Sequencing data were analyzed as described previously ^{31,38}. Briefly, reads were demultiplexed with Basespace FastQ Generation, and the FastQ files were analyzed using custom Python scripts. Paired reads were merged using PEAR (Paired-End reAd mergeR) v0.9.0 ¹¹³. A reference database containing 16S V3-V4 region of each species in the study was created by assembling consensus sequence based on sequencing results of each monospecies. Reads were mapped to the reference database using the mothur v1.40.5 command classify.seqs using the Wang method with bootstrap cutoff value of 60% ^{114,115}. Relative abundance was calculated by dividing the read counts mapped to each organism by the total reads in the sample. Absolute abundance was calculated by multiplying the relative abundance of a given organism by the OD₆₀₀ of the sample. Samples were excluded from further analysis if >1% of the reads were assigned to a species not expected to be in the community due to potential contamination.

### Parameter Estimation of Generalized Lotka-Volterra Models

The generalized Lotka-Volterra (gLV) model is a set of coupled ordinary differential equations that describe species growth as a function of intrinsic growth and pairwise interactions with each constituent community member,

$$\frac{d {x}_{i}}{d t} = {x}_{i} ( {r}_{i} + {\sum }_{j = 1}^{{n}_{s}} {a}_{i j} {x}_{j} ) \qquad ( 3 )$$

where xᵢ is the abundance of species i and nₛ is the total number of species. Model parameters include each species growth rate, denoted as rᵢ, and coefficients that determine how species j affects the growth of species i, denoted as aᵢⱼ. The experimental data used for parameter estimation is the growth of species over time under different inoculation conditions. For monoculture growth data, we use OD₆₀₀ measurements only. For community data, we fit the model to the product of the relative abundance obtained from 16S rRNA gene sequencing and the total OD₆₀₀.

A prior over the parameter distribution is set so that growth rates have a mean of 0.3, self-interaction terms have a mean of −1, and inter-species interaction terms have a mean of −0.1. Given a dataset of measured species abundances over time after inoculating different combinations of species, the model parameters are determined by minimizing a cost function given by a weighted squared difference between model-predicted species abundances and measured abundances and a penalty for deviations from the prior mean. Using the fitted parameter estimates, the covariance of the posterior parameter distribution is approximated as the inverse of the Hessian (matrix of second derivatives) of the cost function with respect to the model parameters. The Expectation-Maximization (EM) algorithm is used to optimize the precision of the prior parameter distribution and the precision of the noise distribution, which collectively determine the degree to which estimated parameters are penalized for deviations from the prior mean ¹¹⁶. Thus, the precision of the prior and noise are hyperparameters that determine the degree of regularization. To evaluate model prediction performance on held-out data, we performed 10-fold cross validation where the degree of regularization was optimized using the EM algorithm and only community samples were subjected to testing (i.e. monoculture data was reserved only for model training). Sulaiman et al. 2024 at the supplementary text in the Supplementary Information has a more detailed description of parameter estimation (section 2-3) and the EM algorithm (section 8).

### Bayesian Experimental Design to Guide Community Experiments

We define an experimental design as a set of unique inoculation conditions, where in each condition each species may be present or absent, and the total inoculation density sums to an OD₆₀₀ of 0.01. We used a Bayesian experimental design approach ⁴⁰ to select experimental conditions that were expected to collectively minimize parameter uncertainty as quantified by the expected Kullback-Leibler (KL) divergence between the posterior parameter distribution and the prior parameter distribution (See Sulaiman et al. 2024 at Equation 20, Section 7 in the supplementary text in Supplementary Information).

Growth of Synthetic Gut Communities Containing Individual C. difficile Isolates

Starter cultures of all C. difficile isolates and commensal gut bacteria were prepared. The cell pellets from starter cultures were collected by centrifugation at 3,000×g for 10 min, and then washed with DM29 media. The washed cell pellets were resuspended into DM29 media to a final OD₆₀₀ of approximately 0.1.

For growth experiments of each of the C. difficile strains with gut bacteria (FIGS. 2D-2E, FIGS. 4A-4D, Sulaiman et al. 2024 at FIG. S6b-c and FIG. S9e), C. difficile and gut bacteria were mixed and cultured in 2-8 member communities. For experiments that will be used to fit gLV models (FIGS. 2D-2E), the community combinations were generated using Bayesian experimental design (Sulaiman et al. 2024 at Table S8, Sulaiman et al. 2024 at supplementary text Section 7 in the Supplementary Information). The monocultures of individual C. difficile strains and each gut species were mixed in equal proportions based on OD₆₀₀ and inoculated into 2 mL 96-deep-well plates (Nest Scientific) containing DM29 supplemented with specific carbohydrate sources (e.g. individual carbohydrates or mixed carbohydrate media) to an initial OD₆₀₀ of 0.01. Depending on the number of species in the community, the initial OD₆₀₀ of each species in the community is 0.01 divided by the total number of species in the community. We used 3 biological replicates for each community. The plates were covered with a gas-permeable seal (Breathe-Easy® sealing membrane) and incubated at 37° C. anaerobically. At specific time points, e.g. after 12 and 24 h of growth, OD₆₀₀ Was measured with a Tecan Infinite Pro F200 microplate reader, and cell pellets were collected for DNA extraction, PCR amplification, and NGS sequencing. For longer-term growth experiments in Sulaiman et al. 2024 at FIG. S9c-d, the communities were cultured for 72 h and an aliquot of the cultures was transferred using 1:20 dilution at 24 and 48 h to observe community assembly over three batch culture growth cycles. For experiments in FIGS. 4A-4D, supernatants of communities at 24 h were collected for toxin quantification using ELISA.

C. difficile Toxin Measurements Using ELISA

Toxin (both TcdA and TcdB) concentrations in C. difficile monocultures or co-cultures, and toxin titer in mice cecal contents were determined by comparison to a standard curve using ELISA (tgcBiomics, Germany). The blank media used for culturing was also included in the assay to measure any background noise. Samples compared for statistical analysis in the same experiment were processed in parallel at the same time using the same batch of ELISA kits to minimize batch-to-batch variations and ensure comparable results.

Growth of C. aerofaciens and B. thetaiotaomicron in the Sterilized Spent Media of Different C. difficile Strains

Starter cultures of C. difficile DSM27147, MS001, MS008, and MS014 were prepared. The cell pellets from starter cultures were collected by centrifugation at 3,000×g for 10 min, and then washed with DM29 media. The washed cell pellets were resuspended into DM29 media to a final OD₆₀₀ of approximately 0.1. Each of the C. difficile strains was inoculated into new culture tubes containing DM29 media supplemented with 5 g/L glucose to an initial OD₆₀₀ of 0.01. Culture tubes were incubated at 37° C. with no shaking. After an incubation time of 24 h, cultures were spun down aerobically at 3,000×g for 20 min and sterile filtered using Steriflip 0.2-μM filters (Millipore-Sigma) before returning to the anaerobic chamber.

Then, starter cultures of C. aerofaciens and B. thetaiotaomicron were prepared. The cell pellets from starter cultures were collected by centrifugation at 3,000×g for 10 min, and then washed with DM29 media. The washed cell pellets were resuspended into DM29 media to a final OD₆₀₀ of approximately 0.1. The CA-BT coculture was inoculated in the sterilized spent media of each C. difficile strain mixed with fresh media (DM29 supplemented with 5 g/L glucose) at an equal ratio to replenish the nutrients. CA and BT were inoculated at an equal initial abundance to a final OD₆₀₀ of 0.01 in 2 mL 96-deep-well plates (Nest Scientific) using gas-permeable seals (BreatheEasy), and incubated at 37° C. with shaking. After 24 h, OD₆₀₀ was measured and the cell pellets were collected for DNA extraction, PCR amplification, and NGS sequencing.

Growth of C. difficile Strains in the Sterilized Spent Media of Gut Bacteria

Starter cultures of commensal gut bacteria were prepared. The cell pellets from starter cultures were collected by centrifugation at 3,000×g for 10 min, and then washed with DM29 media. The washed cell pellets were resuspended into DM29 media to a final OD₆₀₀ of approximately 0.1. Each of the gut bacteria was inoculated into new culture tubes containing the mixed carbohydrates media to an initial OD₆₀₀ of 0.01. Culture tubes were incubated at 37° C. with no shaking. After an incubation time of 24 h, cultures were spun down at 3,000×g for 20 min and sterile-filtered using Steriflip 0.2-μM filters (Millipore-Sigma). The media control (mixed carbohydrates media) was spun down and filtered in parallel with the samples. The pH of the sterilized spent media was adjusted to the same value as the media control.

Next, starter cultures of C. difficile strains were prepared. The cell pellets from starter cultures were collected by centrifugation at 3,000×g for 10 min, and then washed with DM29 media. The washed cell pellets were resuspended into DM29 media to a final OD₆₀₀ of approximately 0.1. The C. difficile strains were inoculated in the sterilized spent media of each strain to a final OD₆₀₀ of 0.01 in 96-well microplates covered with gas-permeable seals (BreatheEasy). The mixed carbohydrates media was used as the control. The plates were incubated at 37° C. with shaking, and OD₆₀₀ was measured every 3 h on a Tecan Infinite Pro F200 microplate reader.

### Transcriptional Profiling

C. difficile DSM27147 monoculture, C. difficile MS001 monoculture, CD DSM-CS coculture, CD MS001-CS coculture, and CD DSM-CH coculture conditions were inoculated from starter cultures into individual culture tubes containing the mixed carbohydrates media. For monoculture conditions, C. difficile was inoculated to an OD₆₀₀ of 0.01. For cocultures, C. difficile and CS or CH were inoculated to an equal ratio (OD₆₀₀ of 0.005 each). The cultures were incubated anaerobically at 37° C. with no shaking for ˜7 h until the culture reached the exponential phase (OD₆₀₀˜0.2). At this point, 1000 μL of the culture was used for an OD₆₀₀ measurement and total DNA extraction for NGS. In parallel, 2000 μL of the culture was used for total RNA extraction for transcriptomics. Next, 4000 μL of RNAprotect (Qiagen) was added to 2000 μL of culture and incubated for 5 min at room temperature. Cultures were then centrifuged at room temperature for 10 min at 3000×g and the supernatant was carefully removed. Cell pellets were immediately subjected to RNA extraction using an acidic phenol bead-beating method. Pellets were resuspended in 500 μL 2× Buffer B (200 mM sodium chloride, 20 mM ethylenediaminetetraacetic acid) and transferred to 2 mL microcentrifuge tubes containing 500 μL Phenol:Chloroform:IAA (125:24:1, pH 4.5) and 210 μL 20% sodium dodecyl sulfate. We performed bead-beating on these samples with acid washed beads (Sigma G1277) for 3 min. All solutions used for RNA extraction were RNAse-free. Samples were centrifuged at 4° C. for 5 min at 7,200×g, and 600 UL of the upper aqueous phase was added to 60 μL 3 M sodium acetate and 660 μL cold isopropanol and chilled on ice for 5 min before freezing for 5 min at −80° C. The samples were centrifuged at 4° C. for 15 min at 18,200×g, the supernatant was decanted, and the pellet was washed with cold 100% ethanol. The pellets were dried in a biosafety cabinet for 15 min and then resuspended in 100 μL RNAse-free water. Samples were purified using RNeasy Mini Kit (Qiagen) and genomic DNA was removed using RNAse-Free DNase Set (Qiagen). Two replicates of each condition were sent to Novogene Corporation Inc (Sacramento, CA, United States of America) for rRNA depletion, cDNA library preparation, and sequencing on an Illumina NovaSeq. The RNA-seq data was de-multiplexed using Illumina's bcl2fastq 2.17 software, where one mismatch was allowed for index sequence identification.

The compressed FASTQ files were quality-checked using the FastQC tool v0.12.1 ¹¹⁷. The BBDuk, BBSplit, and BBMap tools from BBTools suite (v38.42) ¹¹⁸ were used to trim adapters, deplete rRNA, and map the remaining mRNA reads to the reference genomes. For monoculture or cocultures containing C. difficile DSM27147, the reference genome was obtained from GenBank (FN545816.1). For monoculture or cocultures containing C. difficile MS001 isolate, the reference genome was obtained from the whole-genome sequencing data that was assembled and annotated using SPAdes Genome Assembler ⁹². The feature-Counts package v1.6.4 ¹¹⁹ from the SubRead suite was used to map reads to features on the genome and quantify raw counts for each transcript. Reads per kilobase million (RPKM) values were computed using a custom Python script to determine the agreement between biological replicates. The gene expression (represented by RPKM values) showed a strong correlation between biological replicates (Pearson's R=0.95-0.98, P<10E-05) (Sulaiman et al. 2024 at FIG. S16a). The DESeq2 Bioconductor library v4.0.3 ¹²⁰ was used in R v4.0.4 to quantify differential gene expression using a negative binomial generalized linear models with apeglm shrinkage estimator ¹²¹. When calculating RPKM of C. difficile genes in the CD-CS and CD-CH coculture, the “reads mapped” in the denominator was set to the number of reads mapped to the C. difficile genome. Similarly, when quantifying differential gene expression for C. difficile genes in the CD-CS and CD-CH coculture, only reads mapped to the C. difficile genome were provided to DeSeq2. We define differentially expressed genes (DEGs) as those with >2-fold change and an adjusted p-value less than 0.05. The RNA-seq data is accessible in BioProject PRJNA983758.

### Gene Set Enrichment Analysis (GSEA)

GSEA was performed using the GSEA method of the ClusterProfiler R package (v4.2.2) ¹²². KEGG modules for C. difficile were used as gene sets and were supplied as a user-defined annotation with the TERM2GENE field. The analysis was run with the log 2FCs calculated by DeSeq2. The p-value cutoff used was 0.05 and the minimum gene set size used was 3.

### Gnotobiotic Mouse Experiments

All germ-free mouse experiments were performed following protocols approved by the University of Wisconsin-Madison Animal Care and Use Committee. We used 10-week-old C57BL/6 gnotobiotic male mice (wild-type) and a regular diet (Chow diet, Purina, LabDiet 5021). The mice were either provided with normal drinking water or water supplemented with C. difficile-preferred carbohydrates (trehalose, succinate, mannitol, and sorbitol at 1 g/L each) one day before the experiment started.

Bacterial strains were grown at 37° C. anaerobically in Anaerobe Basal Broth (ABB, Oxoid) to stationary phase. C. hiranonis and C. difficile DSM27147 or MS008 strain for oral gavage were diluted to ˜10,000 colony forming units (CFU) per mL and transferred to Hungate tubes (Chemglass) on ice prior to oral gavage. For the 3-member bacterial community for oral gavage, C. aerofaciens, B. uniformis and D. piger were mixed in equal proportions based on OD₆₀₀ and transferred to Hungate tubes (Chemglass) on ice prior to oral gavage. On day 0, either 0.2 mL of C. hiranonis culture or 0.2 mL of the 3-member community was introduced into the mice by oral gavage inside a Biological Safety Cabinet (BSC) and the mice were housed in Sentry SPP (Sealed Positive Pressure) Mouse biocontainment cages for gnotobiotic individually-ventilated caging (IVC) (Allentown Inc.) for the duration of the experiment. After one week, 0.2 mL of C. difficile (˜2,000 CFU) was introduced into the mice by oral gavage. Mice were maintained for a total of two weeks after the first oral gavage with C. hiranonis or 3-member community (day 0). We used 5 biological replicates for the mice orally gavaged with C. difficile DSM27147 and C. hiranonis with unmodified drinking water, and 4 biological replicates for all other groups of mice. Groups of mice with the same experimental condition were co-housed in a single cage. Mice were weighed and fecal samples were collected at specific time points after oral gavage for NGS sequencing. Mice were sacrificed at the end of the experiment or if they displayed a decrease in weight below 75% of their initial weight. Cecal contents from mice that were dead or sacrificed were collected for NGS sequencing.

Genomic DNA Extraction from Fecal and Cecal Samples

The DNA extraction for fecal and cecal samples was performed as described previously with some modifications ¹²³. Fecal samples (˜50 mg) were transferred into solvent-resistant screw-cap tubes (Sarstedt Inc) with 500 μL 0.1 mm zirconia/silica beads (BioSpec Products) and one 3.2 mm stainless steel bead (BioSpec Products). The samples were resuspended in 500 μL of Buffer A (200 mM NaCl (DOT Scientific), 20 mM EDTA (Sigma) and 200 mM Tris·HCl PH 8.0 (Research Products International)), 210 μL 20% SDS (Alfa Aesar) and 500 μL phenol/chloroform/isoamyl alcohol (Invitrogen). Cells were lysed by mechanical disruption with a bead-beater (BioSpec Products) for 3 min twice, while being placed on ice for 1 min in between to prevent overheating. Next, cells were centrifuged for 7 min at 8,000×g at 4° C., and the supernatant was transferred to an Eppendorf tube. We added 60 μL 3M sodium acetate (Sigma) and 600 μL isopropanol (LabChem) to the supernatant and incubated on ice for 1 h. Next, samples were centrifuged for 20 min at 18,000×g at 4° C., and the supernatant was decanted. The harvested DNA pellets were washed once with 500 μL 100% ethanol (Koptec), and the remaining trace ethanol was removed by air drying the samples. Finally, the DNA pellets were resuspended into 300 μL AE buffer (Qiagen). The crude DNA extracts were purified by a Zymo DNA Clean & Concentrator™-5 kit (Zymo Research) prior to PCR amplification and NGS sequencing.

C. difficile CFU Counting from Fecal and Cecal Samples

C. difficile selective plates were prepared by autoclaving C. difficile agar (Oxoid CM0601) and adding defibrinated horse blood (Lampire 7233401, 70 mL/1 L media), norfloxacin (Santa Cruz 215586, 120 μg/mL), moxalactam (Santa Cruz 250419, 320 μg/mL), and erythromycin (Santa Cruz 204742, 100 μg/mL) after the media was cooled to ˜55° C. Immediately following fecal or cecal collection from mice, around 1 μL of fresh fecal samples were taken using an inoculating loop and mixed with PBS. The samples were then serially diluted (1:10 dilution) using PBS. Four dilutions of each sample were spotted on C. difficile selective agar plates, with 2 technical replicates per sample. Plates were incubated at 37° C. for 48 h. At this point, colonies were counted in the dilution spot and contained 5-100 colonies. The CFU/mL for each sample was calculated as the average of the 2 technical replicates times the dilution factor. The lower limit of detection for the assay was 20,000 CFU/mL.

### Statistics and Reproducibility

Most of the statistical analysis in this study compared two different sample groups using a two-sided unpaired Student t-test. Our monoculture growth and community assembly experiments contain 3 biological replicates. Sample sizes were chosen based on limitations of experimental throughput as an increased number of biological replicates would have reduced the number of possible different communities that could be observed.

For fitting monoculture growth data to the logistic growth model that does not capture non-monotonic growth responses and lag phases in growth, we identified time points where the OD₆₀₀ decreased >10% between two sequential time points and omitted all time points beyond this substantial decrease in OD₆₀₀ to eliminate the declining OD₆₀₀ over time. To identify the lag phase, we used a threshold of 120% of the initial OD₆₀₀ (OD₆₀₀ at t=0) and eliminated these time points from the model fitting procedure.

For community growth experiments, individual samples were excluded from the analysis if they were determined to be contaminated by species not intended to be in the sample (>1% of the sample). This cutoff was chosen based on the ratio of the typical background to the typical read depth of our samples. The growth of unique sub-communities to obtain the interaction parameters and generate the interaction networks was performed on a single day with multiple replicates. We performed new experiments to independently validate the inferred interactions (e.g. FIG. 3A, FIGS. 8A-8C, Sulaiman et al. 2024 at FIG. S7d and FIG. S14). To further constrain the inferred interaction parameters from experimental data, we only analyzed parameters (and show edges in the interaction networks in FIGS. 2D and 2E) with absolute values that were significantly constrained to be non-zero based on the Wald test. Since our approach to building our model involved rational selections of sub-communities that fill knowledge gaps in our model, we did not randomize our experimental design. Rather, we used a Bayesian experimental design to select combinations of bacteria that would maximize information content as quantified by the expected Kullback-Leibler divergence between the posterior and prior parameter distributions (see Sulaiman et al. 2024 at supplementary text section 7 in the Supplementary Information).

For toxin measurements using ELISA, all samples that are being compared for statistical analysis (e.g. in FIG. 7 (B), in FIG. 4C and Sulaiman et al. 2024 at FIG. S19a, and in FIG. 6E) were processed in parallel using the same batch of ELISA kits ordered at the same time. We observed that the measured toxin concentration of different strains of C. difficile in monoculture was consistent across different experimental days when cultured in the same media (FIG. 7 (B) and Sulaiman et al. 2024 at FIG. S19a).

For the RNA-seq experiment, we chose 2 biological replicates to generate hypotheses about genes that were significantly changing across conditions. We used DESeq2 to quantify differential gene expressions using a negative binomial generalized linear model with apeglm shrinkage estimator. We observed that the gene expression between the two biological replicates of each RNAseq sample was highly similar (Sulaiman et al. 2024 at FIG. S16a). For statistical analyses, we define differentially expressed genes (DEGs) as those with >2-fold change and an adjusted p-value <0.05 from the Wald test with Benjamini-Hochberg multiple testing correction from DESeq2. We performed follow-up experiments to test the hypotheses generated from the RNA-seq data (e.g. FIGS. 2E-2H, Sulaiman et al. 2024 at FIG. S17).

### Reporting Summary

Further information on research design is available in the Nature Research Reporting Summary linked to this article.

### Data Availability

The whole-genome sequence data of the C. difficile strains have been deposited in the NCBI Sequence Read Archive (SRA) with the code BioProject accession PRJNA902807 [see website provided in Sulaiman et al. 2024]. Mapped growth media and strain-specific genome scale metabolic models in SBML format is publicly available at the website provided in Sulaiman et al. 2024. Next flow pipelines for assembly and metabolic model building is publicly available at the website provided in Sulaiman et al. 2024. The RNA-seq raw data have been deposited in the NCBI Sequence Read Archive (SRA) with the code BioProject accession PRJNA983758 [see website provided in Sulaiman et al. 2024]. The Illumina Sequencing data have been deposited in the NCBI Sequence Read Archive (SRA) with the code BioProject accession PRJNA1134359 [see website provided in Sulaiman et al. 2024], and also available via Zenodo at the website provided in Sulaiman et al. 2024. Source data for all main and supplementary figures are provided with this paper.

### Code Availability

Codes for processing sequencing data, fitting the gLV models, and performing Bayesian experimental design is publicly available at the website provided in Sulaiman et al. 2024.

### REFERENCES

ADDIN EN.REFLIST 1 Cornely, O. A., Miller, M. A., Louie, T. J., Crook, D. W. & Gorbach, S. L. Treatment of first recurrence of Clostridium difficile infection: fidaxomicin versus vancomycin. Clinical infectious diseases 55, S154-S161 (2012).
2 Tsigrelis, C. Recurrent Clostridioides difficile infection: Recognition, management, prevention. Cleveland Clinic Journal of Medicine 87, 347-359 (2020).
3 Song, J. H. & Kim, Y. S. Recurrent Clostridium difficile infection: risk factors, treatment, and prevention. Gut and liver 13, 16 (2019).
4 Kazemian, N. et al. The trans-kingdom battle between donor and recipient gut microbiome influences fecal microbiota transplantation outcome. Scientific reports 10, 18349 (2020).
5 DeFilipp, Z. et al. Drug-resistant E. coli bacteremia transmitted by fecal microbiota transplant. New England Journal of Medicine 381, 2043-2050 (2019).
6 Wang, S. et al. Systematic review: adverse events of fecal microbiota transplantation. PloS one 11, e0161174 (2016).
7 Van Elsas, J. D. et al. Microbial diversity determines the invasion of soil by a bacterial pathogen. Proceedings of the National Academy of Sciences 109, 1159-1164 (2012).
8 Shade, A. et al. Fundamentals of microbial community resistance and resilience. Frontiers in microbiology 3, 417 (2012).
9 Ratner, M. Seres's pioneering microbiome drug fails mid-stage trial. Nature biotechnology 34, 1004-1006 (2016).
10 Feuerstadt, P. et al. SER-109, an oral microbiome therapy for recurrent Clostridioides difficile infection. New England Journal of Medicine 386, 220-229 (2022).
11 Dsouza, M. et al. Colonization of the live biotherapeutic product VE303 and modulation of the microbiota and metabolites in healthy volunteers. Cell Host & Microbe 30, 583-598. e588 (2022).
12 Stubbs, S. L., Brazier, J. S., O'Neill, G. L. & Duerden, B. I. PCR targeted to the 16S-23S rRNA gene intergenic spacer region of Clostridium difficile and construction of a library consisting of 116 different PCR ribotypes. Journal of clinical microbiology 37, 461-463 (1999).
13 Knight, D. R. et al. Major genetic discontinuity and novel toxigenic species in Clostridioides difficile taxonomy. Elife 10 (2021).
14 Scaria, J. et al. Analysis of ultra low genome conservation in Clostridium difficile. PloS one 5, e15147 (2010).
15 Janvilisri, T. et al. Microarray identification of Clostridium difficile core components and divergent regions associated with host origin. Journal of Bacteriology 191, 3881-3891 (2009).
16 Knight, D. R., Elliott, B., Chang, B. J., Perkins, T. T. & Riley, T. V. Diversity and evolution in the genome of Clostridium difficile. Clinical microbiology reviews 28, 721-741 (2015).
17 Juhas, M. Horizontal gene transfer in human pathogens. Critical reviews in microbiology 41, 101-108 (2015).
18 He, M. et al. Evolutionary dynamics of Clostridium difficile over short and long time scales. Proceedings of the National Academy of Sciences 107, 7527-7532 (2010).
19 Brouwer, M. S. et al. Horizontal gene transfer converts non-toxigenic Clostridium difficile strains into toxin producers. Nature communications 4, 1-6 (2013).
20 Kulecka, M. et al. Higher genome variability within metabolism genes associates with recurrent Clostridium difficile infection. BMC microbiology 21, 1-10 (2021).
21 Collins, J., Danhof, H. & Britton, R. A. The role of trehalose in the global spread of epidemic Clostridium difficile. Gut Microbes 10, 204-209 (2019).
22 Collins, J. et al. Dietary trehalose enhances virulence of epidemic Clostridium difficile. Nature 553, 291-294 (2018).
23 Guh, A. Y. et al. Trends in US burden of Clostridioides difficile infection and outcomes. New England Journal of Medicine 382, 1320-1330 (2020).
24 Lessa, F. C. et al. Burden of Clostridium difficile infection in the United States. New England Journal of Medicine 372, 825-834 (2015).
25 Barbut, F. & Petit, J. Epidemiology, risk factors and prevention of Clostridium difficile nosocomial infections. Pathologie-biologie 48, 745-755 (2000).
26 Johnson, S., Adelmann, A., Clabots, C. R., Peterson, L. R. & Gerding, D. N. Recurrences of Clostridium difficile diarrhea not caused by the original infecting organism. The Journal of infectious diseases 159, 340-343 (1989).
27 O'neill, G., Beaman, M. & Riley, T. Relapse versus reinfection with Clostridium difficile. Epidemiology & Infection 107, 627-635 (1991).
28 Tang-Feldman, Y., Mayo, S., Silva Jr, J. & Cohen, S. H. Molecular analysis of Clostridium difficile strains isolated from 18 cases of recurrent Clostridium difficile-associated diarrhea. Journal of clinical microbiology 41, 3413-3414 (2003).
29 Wilcox, M., Fawley, W., Settle, C. & Davidson, A. Recurrence of symptoms in Clostridium difficile infection—relapse or reinfection? Journal of Hospital Infection 38, 93-100 (1998).
30 Buffie, C. G. & Pamer, E. G. Microbiota-mediated colonization resistance against intestinal pathogens. Nature Reviews Immunology 13, 790-801 (2013).
31 Hromada, S. et al. Negative interactions determine Clostridioides difficile growth in synthetic human gut communities. Molecular systems biology 17, e10355 (2021).
32 Pereira, F. C. et al. Rational design of a microbial consortium of mucosal sugar utilizers reduces Clostridioides difficile colonization. Nature Communications 11, 5104 (2020).
33 Aguirre, A. M. et al. Bile acid-independent protection against Clostridioides difficile infection. PLoS Pathogens 17, e1010015 (2021).
34 Girinathan, B. P. et al. In vivo commensal control of Clostridioides difficile virulence. Cell Host & Microbe 29, 1693-1708. e1697 (2021).
35 Kang, J. D. et al. Bile acid 7a-dehydroxylating gut bacteria secrete antibiotics that inhibit Clostridium difficile: role of secondary bile acids. Cell chemical biology 26, 27-34. e24 (2019).
36 Pereira, F. C. et al. Rational design of a microbial consortium of mucosal sugar utilizers reduces Clostridioides difficile colonization. Nature communications 11, 1-15 (2020).
37 Buffie, C. G. et al. Precision microbiome reconstitution restores bile acid mediated resistance to Clostridium difficile. Nature 517, 205-208 (2015).
38 Venturelli, O. S. et al. Deciphering microbial interactions in synthetic human gut microbiome communities. Molecular systems biology 14, e8157 (2018).
39 Baranwal, M. et al. Recurrent neural networks enable design of multifunctional synthetic human gut microbiome dynamics. Elife 11, e73870 (2022).
40 Thompson, J. C., Zavala, V. M. & Venturelli, O. S. Integrating a tailored recurrent neural network with Bayesian experimental design to optimize microbial community functions. PLOS Computational Biology 19, e1011436 (2023).
41 Marino, S., Baxter, N. T., Huffnagle, G. B., Petrosino, J. F. & Schloss, P. D. Mathematical modeling of primary succession of murine intestinal microbiota. Proceedings of the National Academy of Sciences 111, 439-444 (2014).
42 Gonze, D., Coyte, K. Z., Lahti, L. & Faust, K. Microbial communities as dynamical systems. Current opinion in microbiology 44, 41-49 (2018).
43 Qian, Y., Lan, F. & Venturelli, O. S. Towards a deeper understanding of microbial communities: integrating experimental data with dynamic models. Current opinion in microbiology 62, 84-92 (2021).
44 Clark, R. L. et al. Design of synthetic human gut microbiome assembly and butyrate production. Nature communications 12, 1-16 (2021).
45 Ferreyra, J. A. et al. Gut microbiota-produced succinate promotes C. difficile infection after antibiotic treatment or motility disturbance. Cell host & microbe 16, 770-777 (2014).
46 Ghimire, S. et al. Identification of Clostridioides difficile-inhibiting gut commensals using culturomics, phenotyping, and combinatorial community assembly. Msystems 5, e00620-00619 (2020).
47 Theriot, C. M. et al. Antibiotic-induced shifts in the mouse gut microbiome and metabolome increase susceptibility to Clostridium difficile infection. Nature communications 5, 1-10 (2014).
48 Ng, K. M. et al. Microbiota-liberated host sugars facilitate post-antibiotic expansion of enteric pathogens. Nature 502, 96-99 (2013).
49 Camorlinga, M., Sanchez-Rojas, M., Torres, J. & Romo-Castillo, M. Phenotypic characterization of non-toxigenic Clostridioides difficile strains isolated from patients in Mexico. Frontiers in Microbiology 10, 84 (2019).
50 Sebaihia, M. et al. The multidrug-resistant human pathogen Clostridium difficile has a highly mobile, mosaic genome. Nature genetics 38, 779-786 (2006).
51 Mullany, P., Allan, E. & Roberts, A. P. Mobile genetic elements in Clostridium difficile and their role in genome function. Research in microbiology 166, 361-367 (2015).
52 Ghimire, S. et al. Identification of Clostridioides difficile-inhibiting gut commensals using culturomics, phenotyping, and combinatorial community assembly. Msystems 5, 10.1128/msystems. 00620-00619 (2020).
53 Hassall, J., Cheng, J. K. & Unnikrishnan, M. Dissecting individual interactions between pathogenic and commensal bacteria within a multispecies gut microbial community. Msphere 6, 10.1128/msphere. 00013-00021 (2021).
54 Britton, R. A. & Young, V. B. Role of the intestinal microbiota in resistance to colonization by Clostridium difficile. Gastroenterology 146, 1547-1553 (2014).
55 Wasserman, L. All of statistics: a concise course in statistical inference. (Springer Science & Business Media, 2013).
56 Wootton, J. T. Indirect effects and habitat use in an intertidal community: interaction chains and interaction modifications. The American Naturalist 141, 71-89 (1993).
57 Billick, I. & Case, T. J. Higher order interactions in ecological communities: what are they and how can they be detected? Ecology 75, 1529-1543 (1994).
58 Fletcher, J. R., Erwin, S., Lanzas, C. & Theriot, C. M. Shifts in the gut metabolome and Clostridium difficile transcriptome throughout colonization and infection in a mouse model. Msphere 3, e00089-00018 (2018).
59 Battaglioli, E. J. et al. Clostridioides difficile uses amino acids associated with gut microbial dysbiosis in a subset of patients with diarrhea. Science translational medicine 10, eaam7019 (2018).
60 Jenior, M. L., Leslie, J. L., Young, V. B. & Schloss, P. D. Clostridium difficile colonizes alternative nutrient niches during infection across distinct murine gut microbiomes. MSystems 2, e00063-00017 (2017).
61 Theriot, C. M. et al. Antibiotic-induced shifts in the mouse gut microbiome and metabolome increase susceptibility to Clostridium difficile infection. Nature communications 5, 3114 (2014).
62 Karasawa, T., Maegawa, T., Nojiri, T., Yamakawa, K. & Nakamura, S. Effect of arginine on toxin production by Clostridium difficile in defined medium. Microbiology and immunology 41, 581-585 (1997).
63 Ikeda, D. et al. Effect of isoleucine on toxin production by Clostridium difficile in a defined medium. Zentralblatt für Bakteriologie 287, 375-386 (1998).
64 Karlsson, S., Burman, L. G. & Åkerlund, T. Suppression of toxin production in Clostridium difficile VPI 10463 by amino acids. Microbiology 145, 1683-1693 (1999).
65 Karlsson, S., Lindberg, A., Norin, E., Burman, L. G. & Åkerlund, T. Toxins, butyric acid, and other short-chain fatty acids are coordinately expressed and down-regulated by cysteine in Clostridium difficile. Infection and immunity 68, 5881-5888 (2000).
66 Dupuy, B. & Sonenshein, A. L. Regulated transcription of Clostridium difficile toxin genes. Molecular microbiology 27, 107-120 (1998).
67 Wetzel, D. & McBride, S. M. The impact of pH on Clostridioides difficile sporulation and physiology. Applied and environmental microbiology 86, e02706-02719 (2020).
68 Onderdonk, A., Lowe, B. & Bartlett, J. Effect of environmental stress on Clostridium difficile toxin levels during continuous cultivation. Applied and environmental microbiology 38, 637-641 (1979).
69 Wilson, K. H. The microecology of Clostridium difficile. Clinical infectious diseases 16, S214-S218 (1993).
70 Smith, A. B. et al. Enterococci enhance Clostridioides difficile pathogenesis. Nature 611, 780-786 (2022).
71 Majumdar, A. & Govind, R. Regulation of Clostridioides difficile toxin production. Current opinion in microbiology 65, 95-100 (2022).
72 Martin-Verstraete, I., Peltier, J. & Dupuy, B. The regulatory networks that control Clostridium difficile toxin synthesis. Toxins 8, 153 (2016).
73 Powers, D. A., Jenior, M. L., Kolling, G. L. & Papin, J. A. Network analysis of toxin production in Clostridioides difficile identifies key metabolic dependencies. PLOS Computational Biology 19, e1011076 (2023).
74 Voth, D. E. & Ballard, J. D. Clostridium difficile toxins: mechanism of action and role in disease. Clinical microbiology reviews 18, 247-263 (2005).
75 Neumann-Schaal, M., Jahn, D. & Schmidt-Hohagen, K. Metabolism the difficile way: the key to the success of the pathogen Clostridioides difficile. Frontiers in microbiology 10, 219 (2019).
76 Gencic, S. & Grahame, D. A. Diverse energy-conserving pathways in Clostridium difficile: growth in the absence of amino acid Stickland acceptors and the role of the Wood-Ljungdahl pathway. Journal of bacteriology 202, 10.1128/jb. 00233-00220 (2020).
77 Sims, M. D. et al. Safety and tolerability of SER-109 as an investigational microbiome therapeutic in adults with recurrent Clostridioides difficile infection: a Phase 3, open-label, single-arm trial. JAMA Network Open 6, e2255758-e2255758 (2023).
78 Louie, T. et al. VE303, a defined bacterial consortium, for prevention of recurrent Clostridioides difficile infection: a randomized clinical trial. JAMA 329, 1356-1366 (2023).
79 Baldassare, M. A. et al. Butyrate enhances Clostridioides difficile sporulation in vitro. bioRxiv, 2023.2004. 2027.538596 (2023).
80 Jenior, M. L. et al. Novel drivers of virulence in Clostridioides difficile identified via context-specific metabolic network analysis. Msystems 6, e00919-00921 (2021).
81 Werner, M. et al. Prevalence of Clostridioides difficile in Canine Feces and Its Association with Intestinal Dysbiosis. Animals 13, 2441 (2023).
82 Dickson, R. P. The microbiome and critical illness. The Lancet Respiratory Medicine 4, 59-72 (2016).
83 Takáčová, M., Bomba, A., Tóthová, C., Michálová, A. & Turňa, H. Any future for faecal microbiota transplantation as a novel strategy for gut microbiota modulation in human and veterinary medicine? Life 12, 723 (2022).
84 Czepiel, J. et al. Clostridium difficile infection. European Journal of Clinical Microbiology & Infectious Diseases 38, 1211-1221 (2019).
85 Smits, W. K., Lyras, D., Lacy, D. B., Wilcox, M. H. & Kuijper, E. J. Clostridium difficile infection. Nature reviews Disease primers 2, 1-20 (2016).
86 Dieterle, M. G., Rao, K. & Young, V. B. Novel therapies and preventative strategies for primary and recurrent Clostridium difficile infections. Annals of the New York Academy of Sciences 1435, 110-138 (2019).
87 Watson, L. et al. in Open Forum Infectious Diseases. S812 (Oxford University Press).
88 Eggers, S. et al. Wisconsin microbiome study, a cross-sectional investigation of dietary fibre, microbiome composition and antibiotic-resistant organisms: rationale and methods. BMJ open 8, e019450 (2018).
89 Nieto, F. J. et al. The Survey of the Health of Wisconsin (SHOW), a novel infrastructure for population health research: rationale and methods. BMC public health 10, 1-11 (2010).
90 Feng, J. et al. Polysaccharide utilization loci in Bacteroides determine population fitness and community-level interactions. Cell host & microbe 30, 200-215. e212 (2022).
91 Ostrem Loss, E., Thompson, J., Cheung, P. L. K., Qian, Y. & Venturelli, O. S. Carbohydrate complexity limits microbial growth and reduces the sensitivity of human gut communities to perturbations. Nature ecology & evolution 7, 127-142 (2023).
92 Prjibelski, A., Antipov, D., Meleshko, D., Lapidus, A. & Korobeynikov, A. Using SPAdes de novo assembler. Current protocols in bioinformatics 70, e102 (2020).
93 Jain, C., Rodriguez-R, L. M., Phillippy, A. M., Konstantinidis, K. T. & Aluru, S. High throughput ANI analysis of 90K prokaryotic genomes reveals clear species boundaries. Nature communications 9, 1-8 (2018).
94 Tanizawa, Y., Fujisawa, T. & Nakamura, Y. DFAST: a flexible prokaryotic genome annotation pipeline for faster genome publication. Bioinformatics 34, 1037-1039 (2018).
95 Lechner, M. et al. Proteinortho: detection of (co-) orthologs in large-scale analysis. BMC bioinformatics 12, 1-9 (2011).
96 Camacho, C. et al. BLAST+: architecture and applications. BMC bioinformatics 10, 1-9 (2009).
97 Sievers, F. et al. Fast, scalable generation of high-quality protein multiple sequence alignments using Clustal Omega. Molecular systems biology 7, 539 (2011).
98 Lee, M. D. GToTree: a user-friendly workflow for phylogenomics. Bioinformatics 35, 4162-4164 (2019).
99 Letunic, I. & Bork, P. Interactive Tree Of Life (iTOL) v4: recent updates and new developments. Nucleic acids research 47, W256-W259 (2019).
100 Langmead, B. & Salzberg, S. L. Fast gapped-read alignment with Bowtie 2. Nature methods 9, 357-359 (2012).
101 Cury, J., Abby, S. S., Doppelt-Azeroual, O., Néron, B. & Rocha, E. P. in Horizontal Gene Transfer 265-283 (Springer, 2020).
102 Roux, S., Enault, F., Hurwitz, B. L. & Sullivan, M. B. VirSorter: mining viral signal from microbial genomic data. Peer J 3, e985 (2015).
103 Chen, S., Zhou, Y., Chen, Y. & Gu, J. fastp: an ultra-fast all-in-one FASTQ preprocessor. Bioinformatics 34, 1884-1890 (2018).
104 Li, D., Liu, C.-M., Luo, R., Sadakane, K. & Lam, T.-W. MEGAHIT: an ultra-fast single-node solution for large and complex metagenomics assembly via succinct de Bruijn graph. Bioinformatics 31, 1674-1676 (2015).
105 Chklovski, A., Parks, D. H., Woodcroft, B. J. & Tyson, G. W. CheckM2: a rapid, scalable and accurate tool for assessing microbial genome quality using machine learning. Nature Methods 20, 1203-1212 (2023).
106 Chaumeil, P.-A., Mussig, A. J., Hugenholtz, P. & Parks, D. H. GTDB-Tk v2: memory friendly classification with the genome taxonomy database. Bioinformatics 38, 5315-5316 (2022).
107 Hyatt, D. et al. Prodigal: prokaryotic gene recognition and translation initiation site identification. BMC bioinformatics 11, 1-11 (2010).
108 Machado, D., Andrejev, S., Tramontano, M. & Patil, K. R. Fast automated reconstruction of genome-scale metabolic models for microbial species and communities. Nucleic acids research 46, 7542-7553 (2018).
109 Buchfink, B., Reuter, K. & Drost, H.-G. Sensitive protein alignments at tree-of-life scale using DIAMOND. Nature methods 18, 366-368 (2021).
110 Norsigian, C. J. et al. BiGG Models 2020: multi-strain genome-scale models and expansion across the phylogenetic tree. Nucleic acids research 48, D402-D406 (2020).
111 Lieven, C. et al. MEMOTE for standardized genome-scale metabolic model testing. Nature biotechnology 38, 272-276 (2020).
112 Diener, C., Gibbons, S. M. & Resendis-Antonio, O. MICOM: metagenome-scale modeling to infer metabolic interactions in the gut microbiota. MSystems 5, 10.1128/msystems. 00606-00619 (2020).
113 Zhang, J., Kobert, K., Flouri, T. & Stamatakis, A. PEAR: a fast and accurate Illumina Paired-End reAd mergeR. Bioinformatics 30, 614-620 (2014).
114 Wang, Q., Garrity, G. M., Tiedje, J. M. & Cole, J. R. Naive Bayesian classifier for rapid assignment of rRNA sequences into the new bacterial taxonomy. Applied and environmental microbiology 73, 5261-5267 (2007).
115 Schloss, P. D. et al. Introducing mothur: open-source, platform-independent, community-supported software for describing and comparing microbial communities. Applied and environmental microbiology 75, 7537-7541 (2009).
116 Bishop, C. (Springer: Berlin/Heidelberg, Germany, 2004).
117 Andrews, S. (Babraham Bioinformatics, Babraham Institute, Cambridge, United Kingdom, 2010).
118 Bushnell, B. BBMap: a fast, accurate, splice-aware aligner. (Lawrence Berkeley National Lab. (LBNL), Berkeley, CA (United States), 2014).
119 Liao, Y., Smyth, G. K. & Shi, W. featureCounts: an efficient general purpose program for assigning sequence reads to genomic features. Bioinformatics 30, 923-930 (2014).
120 Love, M. I., Huber, W. & Anders, S. Moderated estimation of fold change and dispersion for RNA-seq data with DESeq2. Genome biology 15, 1-21 (2014).
121 Zhu, A., Ibrahim, J. G. & Love, M. I. Heavy-tailed prior distributions for sequence count data: removing the noise and preserving large differences. Bioinformatics 35, 2084-2092 (2019).
122 Wu, T. et al. clusterProfiler 4.0: A universal enrichment tool for interpreting omics data. The Innovation 2, 100141 (2021).
123 Goodman, A. L. et al. Extensive personal human gut microbiota culture collections characterized and manipulated in gnotobiotic mice. Proceedings of the National Academy of Sciences 108, 6252-6257 (2011).

What is claimed is:

1. A composition comprising a bacterial community wherein the bacterial community comprises a purified bacterial strain of each of:
Clostridium hiranonis; and
any one or more of Bacteroides thetaiotaomicron, Bacteroides uniformis, Phocaeicola vulgatus, Clostridium scindens, Collinsella aerofaciens, and Desulfovibrio piger.

2. The composition of claim 1, wherein the bacterial community comprises a purified bacterial strain of each of:
Clostridium hiranonis; and
any one or more or two or more of Phocaeicola vulgatus, Clostridium scindens, Collinsella aerofaciens, and Desulfovibrio piger.

3. The composition of claim 1, wherein the bacterial community comprises a purified bacterial strain of each of Clostridium hiranonis, Clostridium scindens, and Desulfovibrio piger.

4. The composition of claim 1, wherein the composition is devoid of any bacterial strains of B. hydrogenotrophica, E. lenta, or B. hydrogenotrophica and E. lenta.

5. The composition of claim 1, wherein the composition is devoid of any bacterial strains of Clostridium hathewayi, Blautia hansenii, Blautia producta, Blautia producta ATCC 27340, Clostridium bacterium UC5.1-1D4, Blautia coccoides, Eubacterium contortum, Eubacterium fissicatena, Sellimonas intestinalis, Dracourtella massiliensis, Dracourtella massiliensis GDI, Ruminococcus torques, Anaerostipes caccae, Clostridium scindens, Marvinbryantaformexigens, Eisenbergiella tayi, Flavinofractor plautii, Clostridium orbiscindens 1_3_50AFAA, Lachnospiraceae bacterium 7-_I_58FAA, Subdoligranulum, Anaerotruncus colihominis, Anaerotruncus colihominis DSM 17241, Clostridium symbiosum, Clostridium symbiosum WAL-14163, Clostridium bolteae, Clostridium bolteae 90A9, Dorea longicatena, Dorea longicatena CAG: 42, Clostridium innocuum, Erysipelotrichaceae_bacterium_21_3, Blautia wexlerae, Clostridium disporicum, Erysipelatoclostridium ramosum, Pseudoflavinofractor capillosus, Turicibacter sanguinis, Lactobacillus mucosae, Ruminococcus obeum, Megasphaera elsdenii, Acidaminococcus fermentans, Acidaminococcus intestine, Ruminococcus faecis, Bacteroides cellulosilyticus, Anaerostipes hadrus, Eubacterium rectale, Ruminococcus champanellensis, Ruminococcus albus, Bifidobacterium bifidum, Blautia luti, Roseburia faecis, Fusicatenibacter saccharivorans, Roseburia faecis, Blautia faecis, Dorea formicigenerans, Bacteroides ovatus, or any combination of at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, or each of the foregoing.

6. The composition of claim 1, wherein one or more of the purified bacterial strains are in spore form.

7. The composition of claim 1, wherein one or more of the purified bacterial strains are in vegetative form.

8. The composition of claim 1, wherein each of the purified bacterial strains is lyophilized.

9. The composition of claim 1, wherein the bacterial community comprises bacterial strains purified from different sources.

10. The composition of claim 1, wherein the bacterial community inhibits C. difficile toxin production.

11. The composition of claim 1, wherein the bacterial community inhibits C. difficile replication and/or survival.

12. The composition of claim 1, wherein the composition is in the form of a pharmaceutical composition comprising the bacterial community in combination with a pharmaceutically acceptable excipient.

13. The composition of claim 12, wherein the composition is formulated for oral or rectal delivery.

14. The composition of claim 12, wherein the composition is formulated for delivery to the intestine or the colon.

15. The composition of claim 1, wherein the composition is in the form of a food product comprising the bacterial community and a nutrient.

16. A method of treating a C. difficile infection in a subject, comprising administering to the subject a therapeutically effective amount of the composition of claim 1 to treat the C. difficile infection.

17. The method of claim 16, wherein the subject is an asymptomatic C. difficile carrier.

18. The method of claim 16, wherein the subject is administered one or more doses of an antibiotic prior to administration of the composition.

19. The method of claim 16, wherein the subject has not been administered an antibiotic prior to administration of the composition.

20. The method of claim 16, wherein the composition is administered to the subject by oral administration or rectal administration.
